SEQUENCE LISTING (24 sequences)

SEQ ID NO: 1 - DNA, 27 bp, Artificial Sequence
  Synthetic B1-TEV-ADAR1
tattttcagggcatgaatccgcggcag

SEQ ID NO: 2 - DNA, 27 bp, Artificial Sequence
  Synthetic reverse primer B2-ADAR1FLAG
gtcgtccttgtagtctactgggcagag

SEQ ID NO: 3 - DNA, 52 bp, Artificial Sequence
  Synthetic forward primer attB1-TEV
ggggacaagtttgtacaaaaaagcaggctccgagaatctttattttcagggc

SEQ ID NO: 4 - DNA, 57 bp, Artificial Sequence
  Synthetic reverse primer attB2-FLAG
ggggaccactttgtacaagaaagctgggtattacttgtcatcgtcgtccttgtagtc

SEQ ID NO: 5 - DNA, 24 bp, Artificial Sequence
  Synthetic forward primer B1-TEV-ADAR2
tattttcagggcatggatatagaa

SEQ ID NO: 6 - DNA, 27 bp, Artificial Sequence
  Synthetic reverse primer B2-ADAR2FLAG
gtcgtccttgtagtcgggcgtgagtga

SEQ ID NO: 7 - DNA, 44 bp, Artificial Sequence
  Synthetic let-7d -3 FW
gcaagaaaaaaaaaatgggttcctgggaagaggtagtaggttgc

SEQ ID NO: 8 - DNA, 44 bp, Artificial Sequence
  Synthetic let-7d -3 REV
gcaacctactacctcttcccaggaacccattttttttttcttgc

SEQ ID NO: 9 - DNA, 41 bp, Artificial Sequence
  Synthetic let-7d0 FW
aaaaaaaatgggttcctagggagaggtagtaggttgcatag

SEQ ID NO: 10 - DNA, 41 bp, Artificial Sequence
  Synthetic let-7d 0 REV
ctatgcaacctactacctctccctaggaacccatttttttt

SEQ ID NO: 11 - DNA, 35 bp, Artificial Sequence
  Synthetic let-7d +3 FW
aatgggttcctaggaaggggtagtaggttgcatag

SEQ ID NO: 12 - DNA, 35 bp, Artificial Sequence
  Synthetic let-7d +3 REV
ctatgcaacctactaccccttcctaggaacccatt

SEQ ID NO: 13 - DNA, 33 bp, Artificial Sequence
  Synthetic let-7d +59 FW
tgcccacaaggaggtaactatgcgacctgctgc

SEQ ID NO: 14 - DNA, 33 bp, Artificial Sequence
  Synthetic let-7d+59 REV
gcagcaggtcgcatagttacctccttgtgggca

SEQ ID NO: 15 - DNA, 29 bp, Artificial Sequence
  Synthetic JAK2 Forward
gataaagcacacagaaactattcagagtc

SEQ ID NO: 16 - DNA, 23 bp, Artificial Sequence
  Synthetic JAK2 Reverse
agaatattctcgtctccaccaac

SEQ ID NO: 17 - DNA, 20 bp, Artificial Sequence
  Synthetic LIN28B Forward
tgataaaccgagagggaagc

SEQ ID NO: 18 - DNA, 21 bp, Artificial Sequence
  Synthetic LIN28B Reverse
tgtgaattccactggttctcc

SEQ ID NO: 19 - DNA, 20 bp, Artificial Sequence
  Synthetic BCR-ABL Forward
ctccagactgtccacagcat

SEQ ID NO: 20 - DNA, 20 bp, Artificial Sequence
  Synthetic BCR-ABL Reverse
ccctgaggctcaaagtcaga

SEQ ID NO: 21 - DNA, 22 bp, Artificial Sequence
  Synthetic RPL27 Forward
atcgccaagagatcaaagataa

SEQ ID NO: 22 - DNA, 22 bp, Artificial Sequence
  Synthetic RPL27 Reverse
tctgaagacatccttattgacg

SEQ ID NO: 23 - DNA, 19 bp, Artificial Sequence
  Synthetic Lenti-ADARl Forward
aaaaagcaggctccaccat

SEQ ID NO: 24 - DNA, 20 bp, Artificial Sequence
  Synthetic Lenti-ADARl Reverse
acggtgtctgctttccaatc

(19) United States
(12) Patent Application Publication
(10) Pub. No.: US 2019/0247413 A1
(43) Pub. Date: Aug. 15, 2019

(54) COMPOSITIONS AND METHODS FOR TREATING CANCER AND BIOMARKERS TO DETECT CANCER STEM CELL REPROGRAMMING AND PROGRESSION

(71) Applicant: The Regents of the University of California, Oakland, CA (US)

(72) Inventors: Catriona JAMIESON, San Diego, CA (US); Maria Anna ZIPETO, San Diego, CA (US); Leslie ROBERTSON, San Diego, CA (US); Larisa BALAIAN, San Diego, CA (US); Nathaniel Delos SANTOS, San Diego, CA (US); Qingfei JIANG, San Diego, CA (US)

(21) Appl. No.: 16/308,290

(22) PCT Filed: Jun. 8, 2017
(86) PCT No.: PCT/US2017/036651

§ 371 Date: Dec. 7, 2018

Related U.S. Application Data
(60) Provisional application No. 62/347,753, filed on Jun. 9, 2016.

(51) Int. Cl. A61K 31/7076; A61P 35/02; C12N 15/113; C12N 5/095; A61K 31/506; C12Q 1/6886; C12Q 1/6851; C12Q 1/686

(52) U.S. Cl. CPC A61K 31/7076; A61P 35/02; C12N 15/1137; C12N 5/0695; C12Y 305/04; C12Q 1/6886; C12Q 1/6851; C12Q 1/686; A61K 31/506

(57) ABSTRACT

In alternative embodiment, provided are methods and compositions for treating, ameliorating or preventing diseases and conditions, such as cancer, including cancers associated with stem cells such as, without limitation, myelodysplastic syndrome (MDS) and a myeloproliferative neoplasm like chronic myeloid leukemia (CML) or acute myeloid leukemia (AML), and ablating or killing cancer stem cells. In alternative embodiment, provided are a new set of biomarkers to detect leukemia stem cell reprogramming and CML progression. In alternative embodiment, provided are therapeutic targets for treating myelodysplastic syndrome (MDS) and chronic myeloid leukemia (CML) by targeting edited let-7 transcripts.

### RELATED APPLICATIONS

This application claims the benefit of priority to U.S. Provisional Patent Application Ser. No. (USSN) 62/347,753, filed Jun. 9, 2016. The aforementioned application is expressly incorporated herein by reference in its entirety and for all purposes.

### STATEMENT AS TO FEDERALLY SPONSORED RESEARCH

This invention was made with government support under National Institutes of Health (NIH) grant nos. W81XWH-14-1-0121; 2P30CA023100-28; R21CA189705; 5K12GM068524. The government has certain rights in the invention.

### FIELD OF THE INVENTION

The present disclosure relates to the field of oncology, biomarkers, biology and therapeutic targets. In alternative embodiments, provided are methods useful for studying RNA-editing enzymes and their targets, monitoring of disease progression, drug screening, and treatment of cancer. In alternative embodiments, provided are methods and compositions for treating, ameliorating or preventing diseases and conditions, such as cancer, including cancers associated with stem cells such as, without limitation, myelodysplastic syndrome (MDS) and a myeloproliferative neoplasm like chronic myeloid leukemia (CML) or acute myeloid leukemia (AML), and ablating or killing cancer stem cells. In alternative embodiments, provided are a new set of biomarkers to detect leukemia stem cell reprogramming and CML progression. In alternative embodiment, provided are therapeutic targets for treating myelodysplastic syndrome (MDS) and chronic myeloid leukemia (CML) by targeting edited let-7 transcripts. In alternative embodiments, provided are methods for treating, ameliorating, stopping or slowing the progression of, or preventing a cancer or a cancer associated with a stem cell, comprising: administering to a subject in need thereof, or in need of treatment, an agent or combination of agents that inhibit or decrease the expression or activity of: a Janus kinase 2 (JAK2) and a breakpoint cluster region protein (BCR)-Abelson murine leukemia viral oncogene homolog 1 (ABL1) and BCR-ABL1 (a BCR-ABL fusion protein); a JAK2 and a double-stranded RNA-specific adenosine deaminase (also called Adenosine Deaminase Acting on RNA1, or ADAR1); or, a JAK2, an ADAR1 and a BCR-ABL1.

### BACKGROUND OF THE DISCLOSURE

Evidence suggests that Adenosine Deaminase Acting on RNA (ADAR) editases, such as ADAR1 (also called double-stranded RNA-specific adenosine deaminase-1), promote progression and therapeutic resistance of a broad array of human malignancies (Chen et al., 2013; Fumagalli et al., 2015; Han et al., 2015; Jiang et al., 2013; Qi et al., 2014; Qin et al., 2014; Shah et al., 2009; Zipeto et al., 2015). ADAR editases are double stranded (ds) RNA binding proteins that post-transcriptionally deaminate adenosine-to-inosine (A-to-I), most frequently in the context of primate specific Alu repeat sequences that comprise ten percent of the human genome (Kiran and Baranov, 2010; Picardi et al., 2015). By regulating mRNA and microRNA (miRNA) stability, ADARs exhibit wide-ranging effects on embryonic development and stem cell regulation (Han et al., 2015; Liddicoat et al., 2015; Ota et al., 2013; Solomon et al., 2013; Wang et al., 2000). Genetic ADAR1 deletion, particularly impairment of functional RNA editing, induces embryonic lethality in mice by impairing normal hematopoiesis (Guenzland Barlow, 2012; Liddicoat et al., 2015; Wang et al., 2000). Conditional ADAR1 deletion in adult mouse increases interferon signaling that results in hematopoietic stem cells (HSCs) exhaustion (Essers et al., 2009; Hartner et al., 2009). Cumulative human RNA sequencing (RNA-seq) studies demonstrate that deregulated ADAR expression promotes relapse or progression of lobular breast (Shah et al., 2009), hepatocellular (Chen et al., 2013), and esophageal cancer (Qin et al., 2014) as well as transformation of chronic myeloid leukemia (CML) from chronic phase (CP) to a therapy resistant blast crisis (BC) phase (Jiang et al., 2013).

As the first cancer shown to arise in a clonal HSC population, chronic phase (CP) chronic myeloid leukemia (CML) (or CP CML) is initiated by breakpoint cluster region protein (BCR)-Abelson murine leukemia viral oncogene homolog 1 (ABL1), or BCR-ABL1 (a BCR-ABL fusion protein) oncogenic tyrosine kinase expression (Fialkow et al., 1977; Jamieson et al., 2004; Soverini et al., 2015). Progression to blast crisis (BC) phase occurs following malignant reprogramming of committed myeloid progenitors into self-renewing progenitor leukemia stem cell (LSC) (Abrahamsson et al., 2009; Goff et al., 2013; Jamieson et al., 2004; Jiang et al., 2013). While BCR-ABL1-targeted tyrosine kinase inhibitor (TKI) therapy (Druker et al., 1996) has greatly reduced morbidity and mortality in CP CML, therapeutic resistance occurs through BCR-ABL1 mutation and/or amplification that leads to additional genetic and epigenetic modifications that promote progression (Abrahamsson et al., 2009; Goff et al., 2013; Jamieson et al., 2004; Quintas-Cardama et al., 2014; Sawyers, 2010). Increased ADAR1 expression results in myeloid progenitor expansion and conversely, lentiviral shRNA knockdown of ADAR1 prevents malignant progenitor self-renewal in a humanized mouse model of BC CML (Jiang et al., 2013). However, 1) the oncogenic drivers of ADAR1 activity, 2) ADAR1's role in malignant reprogramming of progenitors into self-renewing leukemia stem cells (LSCs), and 3) ADAR1's role in stem cell regulatory miRNA editing as a post-transcriptional mechanism governing self-renewal have not been fully investigated.

### SUMMARY OF THE INVENTION

In alternative embodiments, provided are methods for:
treating, ameliorating, stopping or slowing the progression of, or preventing a cancer or a cancer associated with a stem cell,
inhibiting, decreasing or slowing the progression of a therapeutically responsive (drug responsive) cancer to a therapeutically resistant (drug resistant) cancer,
inhibiting, decreasing or slowing the generation of self-renewing leukemia stem cells (LSCs) or the maintenance of LSCs,
decreasing or inhibiting myelodysplastic syndrome (MDS) or a myeloproliferative neoplasm (MPN) initiation and/or maintenance in inflammatory microenvironments,
inhibiting or decreasing the amount of GSK3β missplicing and increasing degradation of β-catenin, and/or
enhancing let-7 microRNA (miRNA) biogenesis, decreasing adenosine-to-inosine (A-to-I) editing of polycistronic let-7 loci, and/or increasing levels of mature let-7 microRNA (miRNA) levels, comprising:
(a) administering to a subject in need thereof, or in need of treatment, an agent or combination of agents that inhibit or decrease the expression or activity of Janus kinase 2 (JAK2) and:
   (i) breakpoint cluster region protein (BCR)-Abelson murine leukemia viral oncogene homolog 1 (ABL1), or BCR-ABL1 (a BCR-ABL fusion protein),
   (ii) double-stranded RNA-specific adenosine deaminase (also called Adenosine Deaminase Acting on RNA1, or ADAR1), or
   (iii) ADAR1 and BCR-ABL1; or
(b) (i) providing an agent or combination of agents that inhibit or decrease the expression or activity of JAK2 and:
   (1) BCR-ABL1;
   (2) Adenosine Deaminase Acting on RNA1 (ADAR1); or
   (3) ADAR1 and BCR-ABL1,
(ii) administering to a subject in need thereof, or in need of treatment, the agent or combination of agents of (b)(i), thereby:
   treating, ameliorating, stopping or slowing the progression of, or preventing a cancer or a cancer associated with a stem cell,
   inhibiting, decreasing or slowing the progression of a therapeutically responsive (drug responsive) cancer to a therapeutically resistant (drug resistant) cancer,
   inhibiting, decreasing or slowing the generation of self-renewing leukemia stem cells (LSCs) or the maintenance of LSCs,
   decreasing or inhibiting myelodysplastic syndrome (MDS) or a myeloproliferative neoplasm (MPN) initiation and/or maintenance in inflammatory microenvironments,
   inhibiting or decreasing the amount of GSK3β missplicing and increasing degradation of β-catenin, and/or
   enhancing let-7 microRNA (miRNA) biogenesis, decreasing adenosine-to-inosine (A-to-I) editing of polycistronic let-7 loci, and/or increasing levels of mature let-7 microRNA (miRNA) levels.

In alternative embodiments, the cancer or the cancer associated with a stem cell is: (a) myelodysplastic syndrome (MDS) or a myeloproliferative neoplasm (MPN), wherein optionally the myeloproliferative neoplasm (MPN) is chronic myeloid leukemia (CML), a blast crisis (BC) myeloid leukemia (CML) (BC CML), or acute myeloid leukemia (AML), wherein the BC CML is a therapy resistant BC CML; or (b) lobular breast, hepatocellular or esophageal cancer.

In alternative embodiments, the efficacy (or success) of the method is assessed by the detection of:
a decrease in editing efficiency in (or the amount of adenosine-to-inosine (A-to-I) RNA editing of) pri-let-7 microRNA (miRNA) transcripts, or a decrease in the amount of ADAR1-mediated hyper-edited sites in pri-let-7 microRNAs,
a decrease in the adenosine-to-inosine (A-to-I) RNA editing of apolipoprotein B mRNA-editing enzyme catalytic polypeptide-like 3 (APOBEC3),
wherein optionally the amount of A-to-I RNA editing is measured by RNA editing site specific qPCR (RESSqPCR),
wherein optionally the method is considered efficacious or successful if the amount of A-to-I RNA editing, or the amount of ADAR1-mediated hyper-edited sites in pri-let-7 microRNAs, is decreased by at least between about 1% to 50%, or at least about 5% or 10%.

In alternative embodiments of the methods:
(a) the agent or combination of agents that inhibit or decrease the expression or activity of JAK2 comprise: ruxolitinib (or JAKAFI™, or JAKAVI™); lestaurtinib (or CEP-701); pacritinib (or SB-1518); SAR302503 (or TG101348, or N-tert-Butyl-3-{5-methyl-2-[4-(2-pyrrolidin-1-yl-ethoxy)-phenylamino]-pyrimidin-4-ylamino}-benzenesulfonamide); momelotinib (or CYT387, or N-(cyanomethyl)-4-{2-[4-(morpholin-4-yl)anilino]pyrimidin-4-yl}benzamide); AZD1480, or (S)-5-chloro-N2-(1-(5-fluoropyrimidin-2-yl)ethyl)-N4-(5-methyl-1H-pyrazol-3-yl)pyrimidine-2,4-diamine; XL019, or (S)-N-(4-(2-((4-morpholinophenyl)amino)pyrimidin-4-yl)phenyl)pyrrolidine-2-carboxamide; tofacitinib (also known as tasocitinib), or 3-((3R,4R)-4-methyl-3-(methyl(7H-pyrrolo[2,3-d]pyrimidin-4-yl)amino)piperidin-1-yl)-3-oxopropanenitrile, or XELJANZ™, or JAKVINUS™; NVP-BSK805, or 4-(2,6-difluoro-4-(3-(1-(piperidin-4-yl)-1H-pyrazol-4-yl)quinoxalin-5-yl)benzyl)morpholine; or, INCB16562, or 2-(2,6-dichlorophenyl)-1,8-dihydroimidazo[4,5-d]dipyrido[2,3-b:4′,3′-f]azepine;
(b) the agent or combination of agents that inhibit or decrease the expression or activity of BCR-ABL1 comprise: imatinib (or GLEEVEC™, or GLIVEC™); nilotinib (or TASIGN™); dasatinib (or SPRYCEL™, or BMS-354825); bosutinib (or BOSULIF™); pornatinib (or ICLUSIG™, or AP24534); bafetinib or Benzamide, N-(3-((4,5′-bipyrimidin)-2-yl amino)-4-methylphenyl)-4-(((3 S)-3-(dimethylamino)-1-pyrrolidinyl)methyl)-3-(trifluoromethyl)-;4-[[(3S)-3-Dimethylaminopyrrolidin-1-yl]methyl]-N-[4-methyl-3-[(4-pyrimidin-5-ylpyrimidin-2-yl)amino]phenyl]-3-(trifluoromethyl)benzamide; or, a 1,3,4 thiadiazole derivative; or
(c) agent or combination of agents that inhibit or decrease the expression or activity of ADAR1 comprise agents or compositions as described in: WO2013/036867 (PCT/US2012/054307), or U.S. Pat. No. 9,611,330; or WO2015/120197 (PCT/US2015/014686).

In alternative embodiments of the methods:
(a) the agent or combination of agents that inhibit or decrease the expression or activity of JAK2, ADAR1 and/or BCR-ABL1 is or comprises:
   (1) a nucleic acid, and optionally the nucleic acid is an inhibitory nucleic acid comprising: an RNAi inhibitory nucleic acid molecule, a double-stranded RNA (dsRNA) molecule, a microRNA (mRNA), a small interfering RNA (siRNA), an antisense RNA, a short hairpin RNA (shRNA), or a ribozyme capable of capable of inhibiting or decreasing the expression or activity of a JAK2, ADAR1 and/or BCR-ABL1 protein, enzyme, transcript and/or gene,
   (2) a peptide or polypeptide, wherein optionally the polypeptide is or comprises an antibody or fragment thereof or equivalent thereof, capable of specifically binding an JAK2, ADAR1 and/or BCR-ABL1, and is capable of inhibiting or decreasing the activity of a JAK2, ADAR1 and/or BCR-ABL1 protein, enzyme, transcript and/or gene, or
   (3) a small molecule, lipid, saccharide, nucleic acid or polysaccharide capable of inhibiting or decreasing the activity of a JAK2, ADAR1 and/or BCR-ABL1 protein, enzyme, transcript and/or gene;
(b) the compound or composition is formulated as a pharmaceutical composition, or is formulated for administration in vivo; or formulated for enteral or parenteral administration, or for oral, intravenous (IV) or intrathecal (IT) administration, wherein optionally the compound or formulation is administered orally, parenterally, by inhalation spray, nasally, topically, intrathecally, intrathecally, intracerebrally, epidurally, intracranially or rectally;
wherein optionally the formulation or pharmaceutical composition is contained in or carried in a nanoparticle, a particle, a micelle or a liposome or lipoplex, a polymersome, a polyplex or a dendrimer; or
(c) the compound or composition, or the formulation or pharmaceutical composition, is formulated as, or contained in, a nanoparticle, a liposome, a tablet, a pill, a capsule, a gel, a geltab, a liquid, a powder, an emulsion, a lotion, an aerosol, a spray, a lozenge, an aqueous or a sterile or an injectable solution, or an implant.

In alternative embodiments, the nucleic acid capable of inhibiting or decreasing the expression or activity of a JAK2, ADAR1 and/or BCR-ABL1 protein, enzyme, transcript and/or gene comprises or is contained in a nucleic acid construct or a chimeric or a recombinant nucleic acid, or an expression cassette, vector, plasmid, phagemid or artificial chromosome, optionally stably integrated into the cell's chromosome, or optionally stably episomally expressed, and optionally the cell is a cancer cell or a cancer cell line, or a carcinoma cell line or an immortalized cell line.

In alternative embodiments, provided are kits comprising a compound or composition or a formulation or a pharmaceutical composition as provided herein, and optionally comprising instructions on practicing a method as provided herein.

In alternative embodiments, provided are Uses of a compound or composition or a formulation as provided herein in the manufacture of a medicament. In alternative embodiments, provided are Uses of a compound or composition, or a formulation or a pharmaceutical composition as provided herein in the manufacture of a medicament for treating, ameliorating, stopping or slowing the progression of, or preventing a cancer or a cancer associated with a stem cell.

In alternative embodiments, provided are compounds or compositions, or formulations for use in
treating, ameliorating, stopping or slowing the progression of, or preventing a cancer or a cancer associated with a stem cell,
inhibiting, decreasing or slowing the progression of a therapeutically responsive (drug responsive) cancer to a therapeutically resistant (drug resistant) cancer,
inhibiting, decreasing or slowing the generation of self-renewing leukemia stem cells (LSCs) or the maintenance of LSCs,
decreasing or inhibiting myelodysplastic syndrome (MDS) or a myeloproliferative neoplasm (MPN) initiation and/or maintenance in inflammatory microenvironments,
inhibiting or decreasing the amount of GSK3β missplicing and increasing degradation of β-catenin, and/or
enhancing let-7 microRNA (miRNA) biogenesis, decreasing adenosine-to-inosine (A-to-I) editing of polycistronic let-7 loci, and/or increasing levels of mature let-7 microRNA (miRNA) levels,
   wherein the use comprises administering to a subject in need thereof, or in need of treatment, an agent or combination of agents that inhibit or decrease the expression or activity of Janus kinase 2 (JAK2) and,
(i) a breakpoint cluster region protein (BCR)-Abel son murine leukemia viral oncogene homolog 1 (ABL1), or a BCR-ABL1 (a BCR-ABL fusion protein),
(ii) a double-stranded RNA-specific adenosine deaminase (also called Adenosine Deaminase Acting on RNA1, or ADAR1), or
(iii) a ADAR1 and a BCR-ABL1;
   and the compound or composition, or a formulation comprises: an agent or combination of agents that inhibit or decrease the expression or activity of JAK2 and: a ADAR1 and/or a BCR-ABL1.

In alternative embodiments, provided are methods for detecting leukemic progression into blast phase from chronic phase and a method for treating a blast phase leukemia comprising the steps of:
(a) determining if pri-let-7d levels are reduced as compared to a normal control or a previous sample from a patient while in chronic phase; or
(b)
   (i) collecting a blood or serum sample from a patient with leukemia or an individual suspected of having leukemia;
   (ii) isolating mononuclear cells from the blood sample;
   (iii) isolating CD34⁺ cells;
   (iv) isolating RNA from the CD34⁺ cells;
   (v) converting the RNA from step (iv) into cDNA;
   (vi) evaluating miRNA expression using MiScript qPCR array or equivalent; and
   (vii) determining if pri-let-7d levels are reduced as compared to a normal control or a previous sample from the patient while in chronic phase,
wherein a reduction in pri-let-7d levels indicates that the patient is in or entering blast phase leukemia and should be treated or enrolled in a clinical trial,
and optionally, if the pri-let-7d levels are reduced the patient is treated with a combination of drugs or agents comprising: a JAK2 inhibitor, a BCR-ABL-1 inhibitor or a combination of the two; a JAK2 inhibitor, a ADAR1 inhibitor or a combination of the two; or the patient is treated with a combination of drugs as provided herein,
and optionally a reduction of pri-let-7d levels by at least between about 1% to 50%, or at least about 5% or 10%, is considered sufficient to administer the combination of drugs or agents, or is considered sufficient to indicate that the patient is in or entering blast phase leukemia.

In alternative embodiments, provided are methods for treating a patient in blast phase comprising the steps of:
(a) collecting a blood sample from a patient in blast phase;
(b) isolating mononuclear cells from the blood sample;
(c) isolating CD34⁺ cells
(d) isolating RNA from the CD34⁺ cells;
(e) converting the RNA from step (d) into cDNA;
(f) evaluating miRNA expression using MiScript qPCR array; and
(g) determining if pri-let-7d levels are reduced as compared to a normal control or a previous sample from the patient while in chronic phase,
wherein if the pri-let-7d levels are reduced the patient is treated with a JAK2 inhibitor, BCR-ABL-1 inhibitor or a combination of the two or the patient is treated with a JAK2 inhibitor, or ADAR1 inhibitor or a combination of the two.

In alternative embodiments, provided are methods for determining leukemic stem cell generation and/or MPN disease progression using editome signatures of APOBEC3F (A3F) and/or APOBEC3G (A3G) wherein the chronic phase (CP) chronic myeloid leukemia (CML) (or CP CML) and pre-leukemic progenitors or blast crisis (BC) phase have different adenosine-to-inosine (A-to-I) RNA editing signature in A3F and A3G transcripts as compared to a corresponding BC CML and sAML leukemic stem cell,
and optionally if at least 1%, 5% or 10% of the A3F and A3G transcripts differ or if between about 1% and 40% of the A3F and A3G transcripts differ, then a determination of leukemic stem cell generation and/or MPN disease progression can be made, or a progression from CP CIVIL to pre-leukemic progenitors or blast crisis (BC) phase has been made.

In alternative embodiments, provided are methods for detecting edited and unedited RNA transcripts binding to ADAR1 protein comprising:
(a) immunoprecipitating an RNA transcript binding to a ADAR1 protein by Crosslinking Immunoprecipitation (CLIP) with an ADAR1 antibody; and
(b) sequencing the immunoprecipitated RNA transcript and determining how many, or quantifying, how many RNA transcripts are edited or unedited by ADAR1, and/or determining how the RNA transcripts are edited by ADAR1.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The drawings set forth herein are illustrative of exemplary embodiments provided herein and are not meant to limit the scope of the invention as encompassed by the claims.

FIG. 1a-f illustrate data showing that JAK2 Signaling in Progenitor LSCs Increases ADAR1 Expression; as discussed in Example 1, below:

FIG. 1(a) illustrates a Whole transcriptome RNA sequencing analysis of inflammatory KEGG pathway genes in FACS-sorted hematopoietic progenitor cells (Lin⁻CD34⁺CD38⁺) cells from untreated and imatinib-treated (*) primary CP CML (n=8), BC CML (n=9), normal cord blood (n=3) and normal adult peripheral blood (NP, n=3) samples.

FIG. 1(b) graphically illustrates Differentially up-regulated genes in the JAK/STAT/Inflammatory pathway from untreated BC CML (n=6) versus CP CML (n=7) samples.

FIG. 1(c) graphically illustrates Differentially up-regulated genes in the JAK/STAT/Inflammatory pathway from untreated BC CML (n=6) versus Normal (n=6) samples.

FIG. 1(d) graphically illustrates phospho-JAK2 and phospho-STAT5a levels in CD34+ cord blood cells transduced with lenti-BCR-ABL or backbone control (n=3). Graph shows mean ±SEM and statistical analysis by paired t-test.

FIG. 1(e) graphically illustrates Correlation analysis between mRNA expression levels of JAK2 and ADAR1 p150 isoform expression in primary CP (n=3) and BC (n=4) CML progenitors by quantitative RT-PCR (qRT-PCR) relative to RPL27 expression. Graph depicts best-fit line by Pearson correlation analysis.

FIG. 1(f) schematically illustrates Gene set enrichment analysis based on KEGG pathway annotation of JAK/STAT signaling pathway genes in progenitor cells from untreated primary BC compared to CP CML patient samples (p=0.02, Benjamini-Hochberg adjusted). Red and green arrows are indicative of genes that are over-expressed and under-expressed in BC relative to CP, respectively.

FIG. 2a-g illustrate data showing that JAK2 Signaling Promotes ADAR1-mediated adenosine-to-inosine (A-to-I) Editing; as discussed in Example 1, below:

FIG. 2(a) schematically illustrates a diagram of the human wild-type JAK2 lentiviral vector construct (lenti-JAK2).

FIG. 2(b) illustrates a bright-field (BF) and fluorescent (GFP) microscopy showing normal CD34⁺ cord blood cells transduced with lentiviral vector backbone or human JAK2 vector.

FIG. 2(c) graphically illustrates a representative nanoproteomic analysis of total JAK2, phospho-JAK2, phospho-STAT5a and β2 microglobulin (B2M) in 293T control cells transduced with lentiviral human JAK2 (blue) or backbone vector control (green). Peaks represent signal intensity obtained from specific antibodies used.

FIG. 2(d) graphically illustrates a qRT-PCR analysis of human JAK2 mRNA expression levels relative to RPL27 in normal CD34⁺ progenitors transduced with lenti-JAK2 or backbone vector control (n=4).

FIG. 2(e) graphically illustrates qRT-PCR analysis of human ADAR1 isoform levels in normal CD34⁺ cord blood cells transduced with lenti-JAK2 or backbone vector (n=7).

FIG. 2(f) graphically illustrates a correlation analysis of total ADAR1 mRNA expression, measured by qRT-PCR (normalized to HPRT and backbone control), and A-to-I RNA editing of APOBEC3D, measured by RNA editing site specific qPCR (RESSqPCR), in lenti-JAK2-transduced CD34⁺ cord blood cells (n=6). Graph depicts best-fit line by Pearson correlation analysis.

FIG. 2(g) graphically illustrates a Luciferase reporter-based quantification of relative ADAR-mediated A-to-I editing activity in K562 leukemia cells co-transduced with lenti-ADAR1 and lenti-JAK2 following treatment with a JAK2 inhibitor (SAR302503) for 3 hrs at the indicated concentrations. Results represent data from three individual experiments.

FIG. 3a-g graphically illustrate data showing that JAK2 and BCR-ABL1 Impair Let-7 Biogenesis and Enhance LSC Self renewal; as discussed in Example 1, below:

FIG. 3(a) graphically illustrates data from a qRT-PCR analysis of LIN28B transcript levels normalized to RPL27 in normal CD34⁺ progenitors transduced with lenti-JAK2 or backbone vector (n=6).

FIG. 3(b) graphically illustrates data from a miRNA qRT-PCR analysis of mature let-7 family members in normal CD34⁺ cells transduced with lenti-JAK2 or backbone vector (n=3).

FIG. 3(c) graphically illustrates data from a Correlation analysis between LIN28B and ABL1 RNA expression levels in RNA sequencing-based gene expression data (RPKM) from lenti-BCR-ABL transduced normal CD34⁺ cells (n=4). Graph depicts best-fit line and 95% confidence intervals by Pearson correlation analysis.

FIG. 3(d) graphically illustrates data from a mRNA expression levels of LIN28B in normal CD34⁺ progenitors co-transduced with lenti-JAK2 and lenti-BCR-ABL or backbone vector (n=4).

FIG. 3(e) graphically illustrates data showing Self-renewal capacity as measured by percentage of secondary colonies formed after replating primary colonies from CD34⁺ cord blood cells transduced with lenti-JAK2 alone or co-transduced with lenti-BCR-ABL (n=3). (f) Self-renewal capacity as measured by related secondary colonies in CD34⁺ cord blood cells transduced with lenti-BCR-ABL (n=3).

FIG. 3(g) graphically illustrates data showing Self-renewal capacity (normalized to DMSO vehicle) of normal CD34⁺ cord blood cells co-transduced with lenti-JAK2 and lenti-BCR-ABL following treatment SAR302503, dasatinib or the combination (n=3). All graphs in FIG. 3 show mean ±SEM and statistical analyses were calculated using Student's t-test unless otherwise specified.

FIG. 4a-g illustrate how JAK2 and BCR-ABL1 Inhibition Prevents Self-renewal of ADAR1-expressing LSCs; as discussed in Example 1, below:

FIG. 4(a) schematically illustrates an exemplary In vivo experimental design of primary and serial transplantation studies (n>400 mice) with CD34⁺ progenitor cells isolated from BC CML patient samples.

FIG. 4(b) graphically illustrates data of a FACS analysis of GMP (CD34⁺ CD38⁺ CD123⁺ CD45RA⁺ Lin⁻) engraftment in mouse bone marrow (n=5 primary BC CML patient samples) after treatment with vehicle (n=54), SAR302503 (n=59), dasatinib (n=51) or combination (n=52).

FIG. 4(c) graphically illustrates data of a FACS analysis of human progenitor (CD34⁺ CD38⁺ Lin⁻) cell engraftment in mouse bone marrow following serial transplantation of BC CD34+ progenitors from mice treated with vehicle (n=19), SAR302503 (n=19), dasatinib (n=22) or combination (n=22).

FIG. 4(d) graphically illustrates data of a FACS analysis of human GMP engraftment in mouse bone marrow following serial transplantation of BC CD34⁺ progenitor cells from mice treated with vehicle (n=19), SAR302503 (n=19), dasatinib (n=22) or combination (n=22).

FIG. 4(e) graphically illustrates data of a Kaplan Meier plot showing percent survival of secondary recipient mice after serial transplantation of an equal amount (20,000-100,000) of BC CD34⁺ cells isolated from primary transplant recipients treated with vehicle (n=14), SAR302503 (n=13), dasatinib (n=18) or combination (n=18) (p=0.0002 by log-rank test).

FIG. 4(f) graphically illustrates data of a qRT-PCR analysis of relative BCR-ABL1 mRNA levels in equal numbers of CD34⁺ cells isolated from BC CML engrafted mice treated with vehicle (n=10), SAR302503 (n=13), dasatinib (n=11) or combination (n=10) as a measurement of human LSC frequency in bone marrow.

FIG. 4(g) graphically illustrates data of a qRT-PCR analysis of ADAR1 p150 isoform levels in equal numbers of FACS purified human CD34⁺ cells (n=3 individual BC CML patient samples) isolated from bone marrow of mice (3-6 mice per sample per treatment condition) following a 2-day treatment with vehicle, SAR302503, dasatinib or combination. P value is calculated based on 1-way ANOVA. All graphs show means ±SEM; *p<0.05, **p<0.0001 by non-parametric Mann Whitney test unless otherwise specified.

FIG. 5a graphically illustrates data of a RESSqPCR analysis of RNA editing ratio of APOBEC3D and qRT-PCR analysis of ADAR1 mRNA expression levels in K562 leukemia cells transduced with lenti-ADAR1 WT, lenti-ADAR1_{E912A} Mutant or pCDH backbone control (n =5 each).

FIG. 5b graphically illustrates data of a RNA editing ratio of APOBEC3D by RESSqPCR in K562 leukemia cells transduced with ADAR1 WT and ADAR1_{E912A} mutant following 8-aza treatment (n=3). The values are normalized to the editing ratio observed in pCDH control cells.

FIG. 5c graphically illustrates data of a In vitro experimental design used in the following studies. Cord blood CD34, cells co-transduced with lenti-JAK2 and lenti-BCR-ABL, normal or BC CD34, progenitor cells, were treated on SL/M2 stromal cultures with ADAR1 inhibitor 8-azaadenosine (8-Aza, 10-25 nM), JAK2 inhibitor (100 nM of SAR302503), BCR-ABL inhibitor (dasatinib, 10 nM) or combination using same concentrations.

FIG. 5d-e graphically illustrates data of a miRNA expression levels of let-7 family members (d) and ADAR1 expression (e) in CD34, cord blood cells co-transduced with lenti-JAK2 and lenti-BCRABL following treatment (n=3).

FIG. 5f graphically illustrates data of a RNA editing ratio of APOBEC3D by RESSqPCR in CD34, cord blood cells co-transduced with lenti-JAK2 and lenti-BCR-ABL following treatment (n=3).

FIG. 5g graphically illustrates data of an expression of LIN28B of CD34, cord blood cells co-transduced with lenti-JAK2 and lenti-BCR-ABL following treatment (n=3).

FIG. 5h graphically illustrates data of a percentage of secondary colonies formed after replating primary colonies from normal bone marrow or BC CD34, cells treated with different dose of 8-aza (n=3). Graph shows mean ±SD; p values were calculated using ANOVA and Holm-Sidak method.

FIG. 5i graphically illustrates data of a percentage of secondary colonies formed after replating primary colonies from normal bone marrow or BC CML following treatment. Graph shows mean ±SD; p values were calculated using ANOVA and Holm-Sidak method.

FIG. 6a-k illustrate how ADAR1 Enhances Self-Renewal Gene Expression; as discussed in Example 1, below:

FIG. 6(a) illustrates a Gene Set Enrichment Analysis (GSEA) of RNA-seq data revealed the most significantly affected KEGG pathways in normal cord blood CD34⁺ population transduced with lenti-ADAR1 WT (n=3) or vector controls (n=3).

FIG. 6(b) graphically illustrates a GSEA plot showing enrichment of genes in “Signaling Pathways Regulating Pluripotency of Stem Cells” in cord blood CD34⁺ cells transduced with lenti-ADAR1 WT compared with vector controls (n=3).

FIG. 6(c) illustrates a Heatmap depiction of RNA-seq analysis of cord blood CD34⁺ population transduced with lenti-pCDH vector controls (n=3) or lenti-ADAR1 WT (n=3) indicates ADAR editing target genes in stem cell signaling pathways regulating pluripotency of stem cells are significantly differentially expressed (p<0.05, Student's t-test).

FIG. 6(d) graphically illustrates a Volcano plot analysis derived from TPM values showing significantly differentially expressed (p<0.05, Student's t-test) known let-7 target genes (blue) in pluripotency pathway in cord blood CD34⁺ cells transduced with lenti-pCDH vector controls (n=3) or lenti-ADAR1 WT (n=3).

FIG. 6(e) graphically illustrates a Gene set enrichment analysis based on KEGG pathway annotation of signaling pathway regulating stem cell pluripotency in progenitor cells from cord blood CD34⁺ cells transduced with lenti-pCDH vector controls (n=3) or lenti-ADAR1 WT (n=3). Red and green arrows are indicative of genes that are over-expressed and under-expressed in ADAR1 transduced samples compared to pCDH vector controls, respectively.

FIG. 6(f) graphically illustrates a number of colonies formed in primary colony-formation assay by normal CD34⁺ cells transduced with lenti-let-7a or backbone control (n=3). Graph shows average colonies per well and statistical analysis by Student's t-test.

FIG. 6(g) graphically illustrates a percentage of secondary colonies formed after replating primary colonies from CD34⁺ cord blood cells transduced with lenti-let-7a or backbone control (n=3).

FIG. 6(h) illustrates Representative colony pictures of let-7a and backbone transduced CD34+ cord blood cells.

FIG. 6(i) graphically illustrates a number of colonies formed in primary colony-formation assay by normal CD34+ cells transduced with lenti-let-7d or backbone control (n=3).

FIG. 6(j) graphically illustrates a percentage of secondary colonies formed after replating primary colonies from CD34⁺ cord blood cells transduced with lenti-let-7d or backbone control (n=3).

FIG. 6(k) illustrates representative colony pictures of let-7d and backbone transduced CD34+ cord blood cells. All graphs show mean ±SEM and statistical analysis was calculated using the Student's t-test.

FIG. 7a-I illustrate how ADAR1 Enhances Self-Renewal Gene Expression; as discussed in Example 1, below:

FIG. 7(a) RNA sequencing based analysis of let-7 pri-miRNA levels in FACS-sorted hematopoietic progenitor cells (CD34⁺CD38⁺Lin⁻) cells from untreated primary CP and BC CML (n=7 each) samples. n/d=not detectable.

FIG. 7(b) graphically illustrates a miRNA PCR analysis of mature let-7 miRNA levels in primary CP and BC progenitors (n=4 each). Values are normalized to RNU6_2 miRNA expression.

FIG. 7(c) graphically illustrates a percentage self-renewal (replated colonies) from CP CML CD34⁺ progenitors transduced with lenti-ADAR1 WT or vector control (n=3).

FIG. 7(d) graphically illustrates a miRNA PCR analysis of mature let-7d levels in CP CD34⁺ progenitors transduced with lenti-ADAR1 WT or vector control (n=3).

FIG. 7(e) graphically illustrates a miRNA PCR analysis of mature let-7 levels in lenti-ADAR1 WT, lenti-ADAR1E912A Mutant or pCDH backbone transduced normal CD34⁺ cord blood cells (n=5). Values are normalized to RNU6_2 expression.

FIG. 7(f) graphically illustrates a percentage of secondary colonies formed after replating primary colonies from CD34⁺ cord blood cells transduced with lenti-ADAR1 WT, lenti-ADAR1E912A Mutant or backbone vector (n=5).

FIG. 7(g) graphically illustrates a percentage of human CD45⁺ engraftment in bone marrow after transplantation of CD34+ cord blood cells (n=3) transduced with vector control (pCDH), lenti-ADAR1 WT alone, or lenti-ADAR1E912A Mutant into RAG2^{-/-}γc^{-/-} mice (n=5-12 per group).

FIG. 7(h) graphically illustrates a Volcano plot analysis derived from TPM values showing significantly differentially expressed (p<0.05, Student's t-test) known ADAR1 target genes (grey) and self-renewal transcripts (blue) in cord blood CD34⁺ cells transduced with lenti-ADAR1 WT (n=3) compared with lenti-ADAR1E912A Mut (n=3).

FIG. 7(i) illustrates a heat map depiction of RNA-seq analysis of cord blood CD34⁺ population transduced with lenti-ADAR1 WT (n=3) or lenti-ADAR1^{E912A} Mut (n=3) indicates that 38 out of 175 ADAR editing target genes (previously found to be differentially expressed between CP and BC) are significantly differentially expressed (p<0.05, Student's t-test). All graphs show mean±SEM and statistical analysis was calculated using the Student's t-test.

FIG. 8a-g illustrate how ADAR1 Editase Activity Regulates Let-7 Biogenesis; as discussed in Example 1, below:

FIG. 8(a) illustrates a Flowchart that represents the RNA-seq analysis algorithm.

FIG. 8(b) illustrates a ViennaRNA predicted secondary structure changes in let-7d induced by A-to-I editing occurring near DGCR8/DROSHA (yellow) in BC CIVIL 08 and CB9 ADAR1 WT, and predicted DICER cleavage sites in BC CIVIL 07 and CB31 ADAR1 WT (green). The patient samples with the corresponding A-to-I editing sites are labeled next to +3 and +59 cleavage sites.

FIG. 8(c) and FIG. 8(d) graphically illustrate a Depiction of A-to-I editing sites in pri-let-7d, calculated by percentage of guanosine (% G) in (c) primary cord blood, CP CML and BC CML progenitors (n=3-9); or in (d) cord blood CD34⁺ cells transduced with pLOC Backbone, pCDH Backbone, pLOC ADAR1WT, pCDH ADAR1 WT or pCDH-ADAR1 Mut (n=3). RNA editing sites are labeled such that the first base of the mature MIRLET7D is +1; sites seen in only one sample are marked by asterisks. The sites located close to DROSHA/DGCR8 and predicted DICER cleavage sites are labeled with yellow and green squares, respectively.

FIG. 8(e) illustrates a confirmation of the lentiviral constructs of wild-type (WT) unedited or “pre-edited” prilet-7d at +3 ad +59 sites. The arrow points to the A-to-G mutations, reverse sequenced as T-to-C changes.

FIG. 8(f) graphically illustrates data where 293T cells were transfected with WT, +3, +59 or 0 pri-let-7d lentiviral constructs and the mature let-7d expression was measured by RT-qPCR. Experiment was performed in triplicates.

FIG. 8(g) graphically illustrates a Crosslinking RNA Immunoprecipitation (CLIP) in K562 cells stabled transduced with pCDH vector, lenti-ADAR1 WT, and lenti-ADAR1E912A Mutant with an ADAR1 antibody confirms that both ADAR1 WT and ADAR1^{E912A} Mutant are associated with pri-let-7d transcripts. Experiment was performed in triplicates. All graphs show mean ±SEM; p values were calculated using Student's t-test unless otherwise specified.

FIG. 9a-g illustrate how Transcripts in JAK/STAT pathway are upregulated in CIVIL CSCs; as discussed in Example 1, below:

FIG. 9(a) illustrates a whole transcriptome RNA sequencing-based gene expression analysis of inflammatory pathway genes (Qiagen JAK/STAT pathway PCR Array gene list) in FACS-sorted hematopoietic progenitor cells (CD34⁺CD38⁺Lin⁻) cells from untreated and imatinib-treated (*) primary CP CML (n=8), BC CML (n=9), normal cord blood (CB, n=3) and normal adult peripheral blood (NP, n=3) samples.

FIG. 9(b) illustrates a table showing significantly up-regulated genes in JAK/STAT/Inflammatory pathway in untreated BC CML (n=6) versus Normal (n=6) samples (p<0.05 by Mann Whitney adjusted t-test).

FIG. 9(c) graphically illustrates a Nanoproteomic analysis of phospho-JAK2 and phospho-STAT5a levels compared with b2M in CD34⁺ cord blood cells transduced with lenti-BCR-ABL or backbone control (n=3).

FIG. 9(d) illustrates a table showing differentially up-regulated genes in the JAK/STAT/Inflammatory pathway in untreated BC CML samples (n=6) versus CP CML (n=7). P values in red represent genes significantly up-regulated (p<0.05 by Mann Whitney adjusted t-test).

FIG. 9(e) illustrates a table showing fold change of differentially expressed (DE) genes in JAK-STAT KEGG pathway (hsa04630:JAKSTAT signaling pathway) of untreated BC (n=6) and CP samples (n=7) using 1,495 DE genes at a FDR 0.05.

FIG. 9(f) graphically illustrates a Quantitative RT-PCR analysis of JAK2 mRNA levels normalized to human RPL27 in FACS-purified progenitors from normal cord blood (n=9), primary CP CML (n=10) and BC CML (n=12). Graph shows mean and statistical analysis by Student's t-test.

FIG. 9(g) graphically illustrates a Correlation analysis between mRNA expression levels of JAK2 and the ADAR1 p150 isoform in primary BC (n=4) CML progenitors by RNA-seq. Graph depicts a best-fit line by Pearson correlation analysis.

FIG. 10a-f shows that RNA editing luciferase reporter is validated in K562 cell line; as discussed in Example 1, below:

FIG. 10(a) (as a table) and FIG. 10(b) (graphically) illustrate data from a Gene Set Enrichment Analysis (GSEA) of RNA-seq data revealed that JAK-STAT signaling pathways significantly affected in BC CML progenitors (n=6) compared to CP CML progenitors (n=6).

FIG. 10(c) graphically illustrates a Nanoproteomic quantification of total JAK2, p-JAK2, p-STATS in 293T cells transfected with lentiviral control backbone or lenti-JAK2 vectors. Area under the curve was calculated for each protein and normalized to the value of control β2 microglobulin (B2M).

FIG. 10(d) graphically illustrates a Relative luciferase reporter-based activity of ADAR-mediated A-to-I editing in K562 leukemia cells transfected with increasing amounts of plasmid DNA from either pDEST26 ADAR1 or pDEST26 ADAR2 (left panel), and pCDH CMV ADAR1 or pCDH CMV ADAR2 (right panel), compared to backbone control (dotted gray line). pDEST26 is a mammalian express vector and pCDH is lentiviral expression vector. Graphs represent data from 2 individual experiments performed in duplicate.

FIG. 10(e) graphically illustrates a Luciferase reporter-based quantification of relative ADAR-mediated A-to-I editing activity in K562 leukemic cells transduced with lenti-ADAR1, lenti-JAK2+lentiADAR1, or backbone control. Results represent data from three individual experiments.

FIG. 10(f) graphically illustrates a Quantitative RT-PCR analysis of ADAR1 and ADAR2 mRNA expression levels normalized to RPL27 in K562 cells transduced with lenti-ADAR1 or backbone vector (n=3).

FIG 11a-c illustrate that Expression of JAK2 and BCR-ABL1 regulates of let-7 miRNA expression in CML CSCs; as discussed in Example 1, below:

FIG. 11(a) graphically illustrates a Quantitative RT-PCR analysis of JAK2 and BCR-ABL mRNA expression levels in normal CD34+ cells transduced with lenti-JAK2 alone (n=7) or co-transduced with lenti-BCR-ABL (n=4). ** p<0.0005 compared to backbone using Student's t-test.

FIG. 11(b) graphically illustrates a miRNA PCR analysis of mature let-7 family miRNA levels in normal CD34⁺ progenitors transduced with lenti-BCR-ABL with or without SL/M2 stromal co-culture (n=3).

FIG. 11(c) graphically illustrates a miRNA PCR analysis of mature let-7 family miRNA levels in CD34⁺ cord blood cells co-transduced with lenti-JAK2 and lenti-BCR-ABL or backbone vector in SL/M2 stromal co-culture (n=3). Values are normalized to RNU6_2 miRNA expression.

FIG. 12a-b illustrate that selective inhibition of JAK2 leads to ADAR1 downregulation and recuperates let-7 miRNA: FIG. 12(a) graphically illustrates a qRT-PCR analysis of mRNA expression levels of ADAR1 isoforms normalized to RPL27, and, FIG. 12(b) graphically illustrates a miRNA PCR analysis of let-7 miRNA levels in normal CD34⁺ cord blood cells co-transduced withlenti-JAK2 and lenti-BCR-ABL following treatment with JAK2 inhibitor (SAR302503, 100nM and 600nM) on SL/M2 stromal cultures (n=3). All graphs in extended data FIG. 4 show mean±SEM and statistical analysis were calculated using Student's t-test, unless otherwise specified; as discussed in Example 1, below.

FIG. 13a-f illustrate that JAK2 and BCR-ABL1 inhibition prevents CSC propagation; as discussed in Example 1, below:

FIG. 13(a) graphically illustrates representative FACS plots showing human hematopoietic progenitor (CD34⁺CD38⁺Lin⁻) and granulocyte-macrophage progenitor (GMP; CD34⁺CD38⁺CD123⁺CD45RA⁺Lin⁻) cell populations in five primary BC CML patient samples (before transplant).

FIG. 13(b) illustrates a table showing FACS analysis of human hematopoietic stem and progenitor populations in primary BC CML patient samples before xenotransplantation. CMP=common myeloid progenitor, GMP, MEP=megakaryocyte-erythroid progenitor.

FIG. 13(c) illustrates a table showing JAK2, ADAR1 and BCR-ABL mRNA levels in FACS-sorted BC CML progenitor cells before xenotransplantation.

FIG. 13(d) graphically illustrates a FACS analysis of human BC progenitor engraftment in mouse bone marrow following treatment with vehicle (V), SAR302503 (S), dasatinib (D), or combination (C) in humanized BC CML mouse models established with 5 different patient samples. All values are normalized to vehicle mean; statistical analysis is shown by Mann Whitney test for each group; * p<0.05, ** p<0.0001 compared to vehicle-treated controls.

FIG. 13(e) graphically illustrates a FACS analysis of human BC progenitor engraftment (n=5 primary BC CML patient samples) in hematopoietic tissues of RAG2^{-/-}γc^{-/-} mice following treatment with vehicle, SAR302503, dasatinib or combination for liver (n=17-26 mice), spleen (n=51-59), blood (n=47-54) and bone marrow (n=51-59). All values are normalized to vehicle mean; statistical analysis is shown by Mann Whitney test for each group; * p<0.05, ** p<0.0001 compared to vehicle-treated controls.

FIG. 13(f) graphically illustrates a Quantitative nanoproteomic analysis of total JAK2, phospho-JAK2, phospho-CRKL, and phosphoSTAT5a levels in sorted BC progenitor cells from the bone marrow of mice following 2-day treatment with vehicle, SAR302503, dasatinib, or combination.

FIG. 14a-k illustrate that A-to-I editing activity of ADAR1 leads to inhibition of let-7 family miRNA biogenesis; as discussed in Example 1, below:

FIG. 14(a) graphically illustrates a RT-qPCR analysis of BCR-ABL and JAK2 mRNA in K562 cells transduced with pCDH vector control, lenti-ADAR1 WT, and lenti-ADAR1 mutant treated with 8-aza at increasing concentrations (n=3).

FIG. 14(b) illustrates a Quantification of p-STAT5, p-CRKL, and β-actin protein expression in K562 cells transduced with pCDH vector control, lenti-ADAR1 WT, and lenti-ADAR1 mutant treated with 8-aza at increasing concentrations (n=3).

FIG. 14(c) graphically illustrates a qRT-PCR analysis of mRNA expression levels of LIN28B in primary colonies formed by CD34⁺ cord blood cells transduced with lenti-let-7a or backbone vector (n=4 individual CB samples, 30-50 colonies per sample per treatment condition).

FIG. 14(d) graphically illustrates a qRT-PCR analysis of mRNA expression levels of LIN28B in primary colonies formed by CD34⁺ cord blood cells transduced with lenti-let-7d or backbone vector (n=2 individual CB samples, 30-50 colonies per sample per treatment condition).

FIG. 14(e) graphically illustrates a miRNA PCR analysis of mature let-7 miRNA levels following lentiviral pLOC-ADAR1 WT transduction of CD34⁺ cord blood cells (n=3) compared with pLOC backbone vector control.

FIG. 14(f) graphically illustrates a RESSqPCR analysis of RNA editing ratio of APOBEC3D compared with WT (unedited) RNA in K562 leukemia cells transfected with increasing amounts of ADAR1 WT, ADAR1 Mutant or ADAR2 plasmid DNA (n=3). Dotted gray line represents backbone vector control.

FIG. 14(g) graphically illustrates a miRNA PCR analysis of mature let-7 miRNA levels following transduction of K562 cells with lentiADARl WT or lenti-ADAR1 Mutant (n=3).

FIG. 14(h) graphically illustrates a Correlation analysis between total ADAR1 mRNA expression (normalized to RPL27) and RNA editing ratio of APOBEC3D in 293T control cells transduced with lenti-ADAR1 WT or lenti-ADAR1 Mutant (n=3). Graph depicts best-fit lines by Pearson correlation analysis.

FIG. 14(i) graphically illustrates a Quantitative RT-PCR analysis of mRNA expression levels of lenti-ADAR1 expression normalized to RPL27 in normal CD34⁺ cord blood cells transduced with lenti-ADAR1 WT, lenti-ADAR1 Mutant or backbone control (n=5).

FIG. 14(j) graphically illustrates a percentage of live CD34+ cord blood cells, determined using trypan blue exclusion, after transduction with lenti-ADAR1 WT, lenti-ADAR1 Mutant or backbone control (n=5).

FIG. 14(k) illustrates a representative FACS plot of human CD45⁺ engraftment in bone marrow after transplantation of CD34⁺ cord blood cells (n=3) transduced with vector control (pCDH), lenti-ADAR1 WT alone, or lenti-ADAR1 Mutant into RAG2^{-/-}γc^{-/-} mice (n=5-12 per group).

FIG. 15a-e illustrate that RNA editing sites in pri-let-7a and pri-let-7f are detected in CSCs; as discussed in Example 1, below.

FIG. 15(a) schematically illustrates a ViennaRNA Predicted Secondary structure of pri-let-7d in normal peripheral blood (NPB), CP, BC-07 and BC-08 CML patient progenitors subjected to RNA-seq analysis. Putative A-to-G RNA editing sites, base pair probabilities as well as Drosha/DGCR8 and predicted Dicer cleavage sites are shown together with minimum free energy (MFE; kcal/mol) of secondary structures.

FIG. 15(b) schematically illustrates a ViennaRNA Predicted Secondary structure of cord blood CD34⁺ cells transduced with lenti-ADAR1pLOC (CB9 ADAR), lenti-ADAR1 pCDH (CB31 ADAR WT) or-lenti-ADAR1 Mutant, pCDH (CB32ADAR Mut) together with the MFE of each secondary structure.

FIG. 15(c-e) graphically illustrate a Depiction of A-to-I editing calculated by percentage of G nucleotides in pri-let-7f: FIG. 15(c) in progenitors of normal cord blood, CP CIVIL, and BC CIVIL (n=3-9) and in pri-let-7d in FIG. 15(d) cord blood CD34⁺ cells lentivirally transduced with JAK2 and BCR-ABL (n=3); in FIG. 15(e) CIVIL CP CD34⁺ cells lentivirally transduced with pCDH backbone or pCDH ADAR1 WT (n=3). Sites are labeled such that the first base of the maturelet-7 miRNAs is +1; sites seen in only one sample are marked by asterisks (*). The sites located close to DROSHA/DGCR8 and predicted DICER cleavage sites are labeled with yellow and green squares, respectively.

FIG. 16a-b illustrate how A-to-I editing alters pri-let-7d secondary structure; as discussed in Example 1, below:

FIG. 16(a) schematically illustrates a Circos plot depicting A-to-G single nucleotide variations in let-7 cluster regions from the hg19/GRCh37 RNA sequencing-based gene expression analysis of normal CD34⁺ cord blood cells transduced with pLOCADARl WT, pCDH-ADAR1 WT, pCDH-ADAR1 Mutant or backbones control (n=3 each). Overlaid are plus strand Alu sequences (green) and Minus strand Alu sequences (yellow), and labels for the miRNA primary transcripts as described by mirBase.

FIG. 16(b) schematically illustrates a predicted secondary structure of let-7 polycistronic loci. Alu repetitive elements are labeled in pink, brown, yellow and blue based on the sub-family. Zoom-in visualization of pri-let-7d, pri-let-7a, and pri-let-7f secondary structures in also provided.

FIG. 17 graphically illustrates increased A-to-I editing during normal HSPC aging, showing a Volcano Plot of A-to-I (read as G) editing (% G in total nucleotide reads) on known RNA editing coordinates using DARNED and RADAR databases in progenitors of young (n=8) compared to normal aged bone marrow samples (n=8) by RNA-sequencing analysis; as discussed in Example 2, below.

FIG. 18A-C illustrate data showing that transcriptome deregulation distinguishes normal and aged HSC and HPCs: whole transcriptome RNA-seq analysis (gene and isoform FPKMs) of FACS-purified HSC (4 per group) and HPC (6 per group) are shown: FIG. 18A illustrates young and aged bone marrow samples used in RNA-seq studies of normal HSC and HPC; FIG. 18B and FIG. 18C illustrate Log 2 fold change (L2FC) and p values were computed from gene expression data (FPKM+1, aged/young). Profiles of all differentially expressed genes (p<0.05) in human HSC (FIG. 18B) and HPC (FIG. 18C) aging (absolute L2FC>1) are shown; as discussed in Example 2, below.

FIG. 19A-D graphically illustrate data showing aged HSPC myeloid lineage commitment and impaired maintenance in stromal co-culture models; as discussed in Example 2, below:

FIG. 19A graphically illustrates a qRT-PCR analysis of myeloid transcription factor, PU.1, expression in CD34⁺ cells from cord blood (n=3) and aged bone marrow (n=6).

FIG. 19B graphically illustrates of data showing that lentiviral ADAR1 increases PU.1 expression in normal cord blood HSPC-derived colonies (n=3 patients, 15-20 colonies per patient).

FIG. 19C-D graphically illustrate data from stromal monolayers that were established from 3 Young (<35 y/o) and 4 Aged (>65 y/o) normal bone marrow samples, along with the human stromal line HS (HS5/HS27) as a control: Cord blood (CB, n=3) CD34⁺ cells were co-cultured with stroma from young or old bone marrow samples (FIG. 19C) or 3 AML bone marrow (BM) samples (FIG. 19D) for 2 or 4 weeks, or with conditioned media, and plated in survival and self-renewal assays. HS stromal lines were used as controls.

FIG. 20 graphically illustrates of data showing ADAR1 editing activity in myelodysplastic syndrome (MDS); the Volcano Plot shows increased A-to-G editing (% G in total nucleotide reads) sites in MDS (n=4) compared with aged (n=8) HPC by RNA-seq analysis using DARNED and RADAR databases; as discussed in Example 2, below.

FIG. 21A-B graphically illustrate data showing A-to-I editing in commonly deregulated genes in myelodysplastic syndrome (MDS) (A) and cell cycle transcripts (B); A-to-I editing (% G in total nucleotide reads) sites in aged (n=8) compared with MDS progenitor (n=4) RNA-seq using DARNED and RADAR databases; as discussed in Example 2, below.

FIG. 22A-B illustrates Table S1a (FIG. 22A) and S1b (FIG. 22B), which describe data of: a clinical annotation (a) and cytogenetics analysis (b) of primary CML chronic phase (CP) and blast crisis (BC) patient samples; samples were collected prior to treatment except for samples noted in the table that received treatment with hydroxyurea or BCR-ABL1 tyrosine kinase inhibitor therapy; NA=data not available.

FIG. 23 illustrates Tables S4a-b data shows significantly affected KEGG pathway genes in normal cord blood CD34+ progenitors transduced with Tables S(a) lenti-ADAR1 Mut (n=3) compared to vector controls (n=3), and Tables S(b) lenti-ADAR1 WT (n=3) compared to lenti-ADAR1 Mut (n=3), analyzed by RNA-seq and Gene Set Enrichment Analysis (GSEA)

FIG. 24 illustrates Table S5, which shows data from an RNA-seq analysis of differentially expressed self-renewal genes in ADAR1 WT compared with ADAR1 Mut transduced CD34+ cord blood samples.

FIG. 25 illustrates a Table describing quantitative RT-PCR primer sequences for amplifying the transcripts as set forth in the Table.

Like reference symbols in the various drawings indicate like elements.

### DETAILED DESCRIPTION

In alternative embodiments, provided are methods for treating, ameliorating, stopping or slowing the progression of, or preventing a cancer or a cancer associated with a stem cell, comprising: administering to a subject or individual in need thereof, or in need of treatment, an agent or combination of agents, e.g., a pharmaceutical, that inhibit or decrease the expression or activity of: a Janus kinase 2 (JAK2) and a breakpoint cluster region protein (BCR)-Abelson murine leukemia viral oncogene homolog 1 (ABL1) and BCR-ABL1 (a BCR-ABL fusion protein); a JAK2 and a double-stranded RNA-specific adenosine deaminase (also called Adenosine Deaminase Acting on RNA1, or ADAR1); or, a JAK2, an ADAR1 and a BCR-ABL1. In alternative embodiments, the cancer or the cancer associated with a stem cell is: myelodysplastic syndrome (MDS) or a myeloproliferative neoplasm (MPN), wherein optionally the myeloproliferative neoplasm (MPN) is chronic myeloid leukemia (CML), a blast crisis (BC) myeloid leukemia (CML) (BC CML), or acute myeloid leukemia (AML), wherein the BC CML is a therapy resistant BC CML.

In alternative embodiments, the disclosure herein addresses an unmet need to identify novel biomarkers of oncogenic transformation of pre-malignant progenitors that will aid in the development of human cancer stem cell- (CSC-) or leukemia stem cell- (LSC-) targeted diagnostic and therapeutic strategies capable of predicting and preventing progression and of, e.g., myeloproliferative neoplasms (MPNs) to acute myeloid leukemia (AML). Recoding of RNA by ADAR editases, e.g., ADAR1, is an essential driver of therapeutic resistance, relapse and progression in lobular breast, hepatocellular, esophageal cancer and hallmark myeloproliferative neoplasms (MPNs) like chronic myeloid leukemia (CML).

In alternative embodiments, disclosed herein are a new set of biomarkers to detect leukemia stem cell (LSC) reprogramming and chronic myeloid leukemia (CML) progression, and new therapeutic targets for treating myelodysplastic syndrome (MDS) or a myeloproliferative neoplasm (MPN), wherein optionally the myeloproliferative neoplasm (MPN) is chronic myeloid leukemia (CML), a blast crisis (BC) myeloid leukemia (CML) (BC CML), or acute myeloid leukemia (AML).

Data disclosed herein indicates that blast crisis CML patient transcriptomes encompass hyper-edited (adenosine-to-inosine (A-to-I) RNA editing) sites in pri-let-7 microRNAs induced by the activation of ADAR1. Such hyper-editing is not observed in normal patients and chronic phase (CP) CML patients, suggesting these events are novel biomarkers for predication of disease progression and therapeutic targets by targeting the edited let-7 transcripts.

Also, disclosed herein is evidence that a RNA editor, ADAR1, may edit the DNA editor apolipoprotein B mRNA-editing enzyme catalytic polypeptide-like 3 (APOBEC3s) in the therapeutic resistance population of CML LSCs, which directly link RNA editing to DNA mutagenesis and leukemia relapse. These data suggest the adenosine-to-inosine (A-to-I) editomes of APOBEC3s are biomarkers raised during disease progression due to LSC generation.

In alternative embodiments, disclosed herein is a therapeutic method of treating subjects in need of treatment with a Janus kinase 2 (JAK2) inhibitor in combination with: a breakpoint cluster region protein (BCR)-Abelson murine leukemia viral oncogene homolog 1 (ABL1), or BCR-ABL1 (a BCR-ABL fusion protein) and/or double-stranded RNA-specific adenosine deaminase (also called Adenosine Deaminase Acting on RNA1, or ADAR1) inhibitor. Since BCR-ABL1 and JAK2 signaling converges on ADAR1 activation and the downstream activation of LIN28B by editing of let-7 microRNAs, the combination therapy of JAK2 and ADAR1 inhibition, or BCR-ABL and JAK2 inhibition, provide more effective treatment and complete elimination of leukemia stem cells in subjects with ADAR1 activation. The efficacy of these combination treatments can be assessed by the detection of editing efficiency in pri-let-7 transcripts.

In alternative embodiments, JAK2 inhibitors useful in the methods disclosed herein include, without limitation, JAK2 comprise: ruxolitinib (or JAKAFI™, or JAKAVI™); lestaurtinib (or CEP-701); pacritinib (or SB-1518); SAR302503 (or TG101348, or N-tert-Butyl-3-{5-methyl-2-[4-(2-pyrrolidin-1-yl-ethoxy)-phenylamino]-pyrimidin-4-ylamino}-benzenesulfonamide); momelotinib (or CYT387, or N-(cyanomethyl)-4-{2-[4-(morpholin-4-yl)anilino]pyrimidin-4-yl}benzamide); AZD1480, or (S)-5-chloro-N2-(1-(5-fluoropyrimidin-2-yl)ethyl)-N4-(5-methyl-1H-pyrazol-3-yl)pyrimidine-2,4-diamine; XL019, or (S)-N-(4-(2-((4-morpholinophenyl)amino)pyrimidin-4-yl)phenyl)pyrrolidine-2-carboxamide; tofacitinib (also known as tasocitinib), or 3-((3R,4R)-4-methyl-3-(methyl(7H-pyrrolo[2,3-d]pyrimidin-4-yl)amino)piperidin-1-yl)-3-oxopropanenitrile, or XELJANZ™, or JAKVINUS™; NVP-BSK805, or 4-(2,6-difluoro-4-(3-(1-(piperidin-4-yl)-1H-pyrazol-4-yl)quinoxalin-5-yl)benzyl)morpholine; or, INCB16562, or 2-(2,6-dichlorophenyl)-1,8-dihydroimidazo[4,5 -d]dipyrido[2,3 -b :4′,3′-f]azepine.

In alternative embodiments, BCR-ABL tyrosine kinase inhibitors useful in the methods disclosed herein include, without limitation, imatinib, nilotinib, dasatinib, bosutinib, pornatinib, bafetinib, and 1,3,4 thiadiazole derivatives.

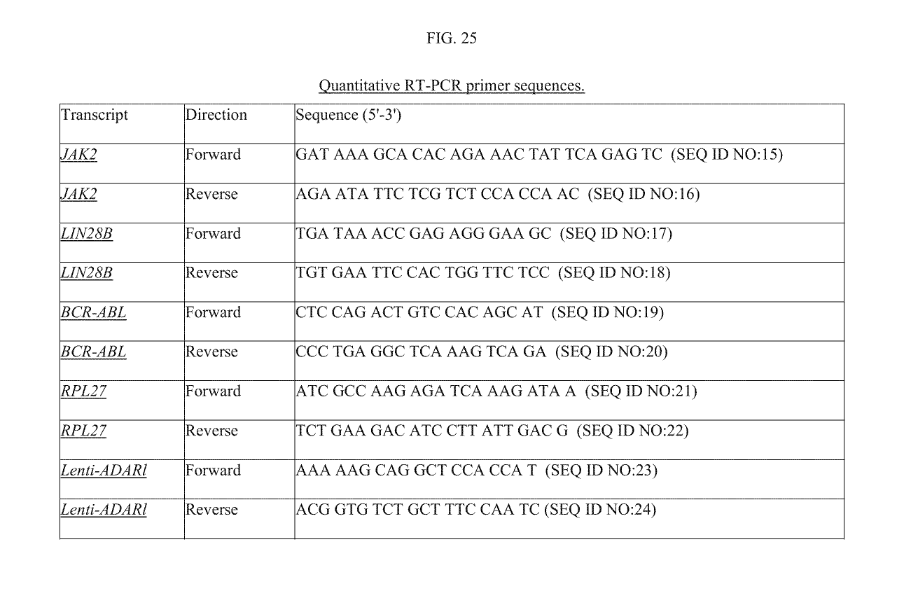

In alternative embodiments, ADAR1 inhibitors comprise agents or compositions as described in: WO2013/036867 (PCT/US2012/054307), or U.S. Pat. No. 9,611,330; or WO2015/120197 (PCT/US2015/014686). Useful ADAR1 inhibitors include derivatives of 8-azaadenosine, e.g., see FIG. 25 of WO2015/120197 and EP0066918, which is incorporated herein by reference.

Also provided herein are combination therapies using one or more compounds or compositions disclosed herein, or pharmaceutically acceptable salts, solvates or hydrates thereof, in combination with other pharmaceutically active agents for the treatment of the diseases and disorders described herein.

In one embodiment, such additional pharmaceutical agents include one or more chemotherapeutic agents, anti-proliferative agents, hypomethylating agents, topoisomerase I inhibitors, interferon alpha, anti-inflammatory agents, radioactive phosphorus, immunomodulatory agents or immunosuppressive agents. Such agents that can be used in the therapeutic methods disclosed herein include azacitidine, prednisone, androgens, EPO, thalidomide, hydroxyurea, anagrelide, busulfan, 2-CDA Lenalidemide. Still other agents that can be combined include antifibrotics, such as PRM-151 (or recombinant human serum amyloid P/pentraxin 2) and simtuzumab (also called GS-6624, a humanized monoclonal antibody designed for the treatment of fibrosis that binds to LOXL2).

### Antisense Inhibitory Nucleic Acid Molecules

In alternative embodiments, JAK2-, ADAR1- and/or BCR-ABL1-inhibiting pharmaceutical compositions and formulations methods as provided herein are administered to an individual in need thereof in an amount sufficient to practice methods as provided herein, e.g., for: treating, ameliorating, stopping or slowing the progression of, or preventing a cancer or a cancer associated with a stem cell; inhibiting, decreasing or slowing the progression of a therapeutically responsive (drug responsive) cancer to a therapeutically resistant (drug resistant) cancer; inhibiting, decreasing or slowing the generation of self-renewing leukemia stem cells (LSCs) or the maintenance of LSCs; decreasing or inhibiting myelodysplastic syndrome (MDS) or a myeloproliferative neoplasm (MPN) initiation and/or maintenance in inflammatory microenvironments; inhibiting or decreasing the amount of GSK3β missplicing and increasing degradation of β-catenin; and/or, enhancing let-7 microRNA (miRNA) biogenesis, decreasing adenosine-to-inosine (A-to-I) editing of polycistronic let-7 loci, and/or increasing levels of mature let-7 microRNA (miRNA) levels.

In alternative embodiments, compositions used to practice methods as provided herein comprise inhibitory nucleic acids, e.g., an antisense morpholino oligonucleotide (MO), an miRNA, an siRNA and the like.

In alternative embodiments, compositions and methods as provided herein comprise use of an inhibitory nucleic acid molecule or an antisense oligonucleotide inhibitory to activity and/or expression of a JAK2-, ADAR1- and/or BCR-ABL1 transcript or gene. In alternative embodiments, compositions and methods as provided herein comprise use of an inhibitory nucleic acid molecule or antisense oligonucleotide inhibitory to expression of JAK2, ADAR1 and/or BCR-ABL1 encoding nucleic acids, comprising: an RNAi inhibitory nucleic acid molecule, a double-stranded RNA (dsRNA) molecule, a small interfering RNA (siRNA), a microRNA (miRNA) and/or a short hairpin RNA (shRNA), or a ribozyme.

Naturally occurring or synthetic nucleic acids can be used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata (1997) Toxicol. Appl. Pharmacol. 144:189-197; Antisense Therapeutics, ed. Agrawal (Humana Press, Totowa, N.J., 1996). Antisense oligonucleotides having synthetic

DNA backbone analogues can also include phosphoro-dithioate, methylphosphonate, phosphoramidate, alkyl phosphotriester, sulfamate, 3′-thioacetal, methylene (methylimino), 3′-N-carbamate, and morpholino carbamate nucleic acids.

### RNA interference (RNAi)

In alternative embodiments, provided are RNAi inhibitory nucleic acid molecules capable of decreasing or inhibiting expression of one or a set of JAK2-, ADAR1- and/or BCR-ABL1-transcripts or proteins, e.g., the transcript (mRNA, message) or isoform or isoforms thereof. In one aspect, the RNAi molecule comprises a double-stranded RNA (dsRNA) molecule. The RNAi molecule can comprise a double-stranded RNA (dsRNA) molecule, e.g., siRNA, miRNA (microRNA) and/or short hairpin RNA (shRNA) molecules.

In alternative aspects, the RNAi is about 11, 12, 13, 14, 15, 16, 17, 18, 19, 20, 21, 22, 23, 24, 25 or more duplex nucleotides in length. While the methods provided herein are not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi, e.g., siRNA for inhibiting transcription and/or miRNA to inhibit translation, is the breaking of a double-stranded RNA (dsRNA) matching a specific gene sequence into short pieces called short interfering RNA, which trigger the degradation of mRNA that matches its sequence.

In one aspect, intracellular introduction of the RNAi (e.g., miRNA or siRNA) is by internalization of a target cell specific ligand bonded to an RNA binding protein comprising an RNAi (e.g., microRNA) is adsorbed. The ligand can be specific to a unique target cell surface antigen. The ligand can be spontaneously internalized after binding to the cell surface antigen. If the unique cell surface antigen is not naturally internalized after binding to its ligand, internalization can be promoted by the incorporation of an arginine-rich peptide, or other membrane permeable peptide, into the structure of the ligand or RNA binding protein or attachment of such a peptide to the ligand or RNA binding protein. See, e.g., U.S. Patent App. Pub. Nos. 20060030003; 20060025361; 20060019286; 20060019258. In one aspect, provided are lipid-based formulations for delivering, e.g., introducing nucleic acids used in methods as provided herein, as nucleic acid-lipid particles comprising an RNAi molecule to a cell, see .g., U.S. Patent App. Pub. No. 20060008910.

Methods for making and using RNAi molecules, e.g., siRNA and/or miRNA, for selectively degrade RNA are well known in the art, see, e.g., U.S. Pat. Nos. 6,506,559; 6,511,824; 6,515,109; 6,489,127.

Methods for making expression constructs, e.g., vectors or plasmids, from which an inhibitory polynucleotide (e.g., a duplex siRNA) is transcribed are well known and routine. A regulatory region (e.g., promoter, enhancer, silencer, splice donor, acceptor, etc.) can be used to transcribe an RNA strand or RNA strands of an inhibitory polynucleotide from an expression construct. When making a duplex siRNA inhibitory molecule, the sense and antisense strands of the targeted portion of the targeted IRES can be transcribed as two separate RNA strands that will anneal together, or as a single RNA strand that will form a hairpin loop and anneal with itself. For example, a construct targeting a portion of a gene, e.g., a RNA helicase and/or an autophagy pathway coding sequence or transcriptional activation sequence, is inserted between two promoters (e.g., mammalian, viral, human, tissue specific, constitutive or other type of promoter) such that transcription occurs bidirectionally and will result in complementary RNA strands that may subsequently anneal to form an inhibitory siRNA used to practice methods as provided herein.

Alternatively, a targeted portion of a gene, coding sequence, promoter or transcript can be designed as a first and second antisense binding region together on a single expression vector; for example, comprising a first coding region of a targeted gene in sense orientation relative to its controlling promoter, and wherein the second coding region of the gene is in antisense orientation relative to its controlling promoter. If transcription of the sense and antisense coding regions of the targeted portion of the targeted gene occurs from two separate promoters, the result may be two separate RNA strands that may subsequently anneal to form a gene-inhibitory siRNA used to practice methods as provided herein.

In another aspect, transcription of the sense and antisense targeted portion of the targeted gene is controlled by a single promoter, and the resulting transcript will be a single hairpin RNA strand that is self-complementary, i.e., forms a duplex by folding back on itself to create a gene-inhibitory siRNA molecule. In this configuration, a spacer, e.g., of nucleotides, between the sense and antisense coding regions of the targeted portion of the targeted gene can improve the ability of the single strand RNA to form a hairpin loop, wherein the hairpin loop comprises the spacer. In one embodiment, the spacer comprises a length of nucleotides of between about 5 to 50 nucleotides. In one aspect, the sense and antisense coding regions of the siRNA can each be on a separate expression vector and under the control of its own promoter.

### Inhibitory Ribozymes

In alternative embodiment, compositions and methods as provided herein comprise use of ribozymes capable of binding and inhibiting, e.g., decreasing or inhibiting, expression of one or a set of JAK2-, ADAR1- and/or BCR-ABL1-transcripts or proteins, or isoform or isoforms thereof.

These ribozymes can inhibit a gene's activity by, e.g., targeting a genomic DNA or an mRNA (a message, a transcript). Strategies for designing ribozymes and selecting a gene-specific antisense sequence for targeting are well described in the scientific and patent literature, and the skilled artisan can design such ribozymes using these reagents. Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it can be released from that RNA to bind and cleave new targets repeatedly.

### Pharmaceutical Compositions

In alternative embodiments, provided are pharmaceutical compositions and formulations for practicing the methods as provided herein, where in alternative embodiments the pharmaceutical compositions and formulations comprise JAK2-, ADAR1- and BCR-ABL1- inhibitory compositions such as inhibitory small molecules, proteins (e.g., antibodies), lipids, saccharides, or nucleic acids (as discussed above).

In alternative embodiments, compositions used to practice the methods as provided herein are formulated with a pharmaceutically acceptable carrier. In alternative embodiments, the pharmaceutical compositions used to practice the methods as provided herein can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Therapeutic agents used to practice the methods as provided herein can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions used to practice the methods as provided herein include those suitable for oral/ nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Pharmaceutical formulations used to practice the methods as provided herein can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, geltabs, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores.

Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations used to practice the methods as provided herein can also be used orally using, e.g., push-fit capsules made of gelatin, as well as soft, sealed capsules made of gelatin and a coating such as glycerol or sorbitol. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., a composition used to practice the methods as provided herein) in admixture with excipients suitable for the manufacture of aqueous suspensions. Such excipients include a suspending agent, such as sodium carboxymethylcellulose, methylcellulose, hydroxypropyl-methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals are particularly useful for administration hydrophobic active agents used to practice the methods as provided herein. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations as provided herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In practicing methods provided herein, the pharmaceutical compounds can also be administered by in intranasal, intraocular and intravaginal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In practicing methods provided herein, the pharmaceutical compounds can be delivered by transdermally, by a topical route, formulated as applicator sticks, solutions, suspensions, emulsions, gels, creams, ointments, pastes, jellies, paints, powders, and aerosols.

In practicing methods provided herein, the pharmaceutical compounds can also be delivered as nanoparticles or microspheres for slow release in the body. For example, nanoparticles or microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In practicing methods provided herein, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

The pharmaceutical compounds and formulations used to practice the methods as provided herein can be lyophilized. Provided are a stable lyophilized formulation comprising a composition as provided herein, which can be made by lyophilizing a solution comprising a pharmaceutical as provided herein and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

The compositions and formulations used to practice the methods as provided herein can be delivered by the use of liposomes or nanoliposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

The formulations used to practice the methods as provided herein can be administered for prophylactic and/or therapeutic treatments. In therapeutic applications, compositions are administered to a subject already suffering from a condition, infection or disease in an amount sufficient to cure, alleviate or partially arrest the clinical manifestations of the condition, infection or disease and its complications (a “therapeutically effective amount”). For example, in alternative embodiments, pharmaceutical compositions as provided herein are administered in an amount sufficient to for e.g., treating, ameliorating, stopping or slowing the progression of, or preventing a cancer or a cancer associated with a stem cell; inhibiting, decreasing or slowing the progression of a therapeutically responsive (drug responsive) cancer to a therapeutically resistant (drug resistant) cancer; or inhibiting, decreasing or slowing the generation of self-renewing leukemia stem cells (LSCs) or the maintenance of LSC. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, an exemplary pharmaceutical formulation for oral administration of compositions used to practice the methods as provided herein can be in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods as provided herein can further comprise co-administration with other drugs or pharmaceuticals, e.g., compositions for treating cancer, septic shock, infection, fever, pain and related symptoms or conditions. For example, the methods and/or compositions and formulations as provided herein can be co-formulated with and/or co-administered with antibiotics (e.g., antibacterial or bacteriostatic peptides or proteins), particularly those effective against gram negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof.

### Nanoparticles, Nanolipoparticles and Liposomes

Also provided are liposomes, nanoparticles, nanolipoparticles, vesicles and liposomal membranes comprising compounds, pharmaceutical compositions or formulations used to practice the methods as provided herein, e.g., to deliver the compounds, pharmaceutical compositions or formulations to mammalian cells in vivo, in vitro or ex vivo. In alternative embodiments, these liposomes, nanoparticles, nanolipoparticles, vesicles and liposomal membranes are designed to target specific molecules, including biologic molecules, such as polypeptides, including cell surface polypeptides, e.g., for targeting a desired cell type, e.g., a myocyte or heart cell, and endothelial cell, and the like.

Provided are multilayered liposomes comprising compounds used to practice methods as provided herein, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods as provided herein.

Liposomes can be made using any method, e.g., as described in Park, et al., U.S. Pat. Pub. No. 20070042031, including method of producing a liposome by encapsulating an active agent (e.g., an inhibiting nucleic acid, small molecule or polypeptide), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods as provided herein comprise a substituted ammonium and/or polyanions, e.g., for targeting delivery of a compound (e.g., JAK2-, ADAR1- and BCR-ABL1-inhibitory small molecules, nucleic acids and polypeptides) used to practice methods as provided herein to a desired cell type (e.g., a stem cell, a particular type of blood cell, an endothelial cell, a cancer cell, or any tissue in need thereof), as described e.g., in U.S. Pat. Pub. No. 20070110798.

Provided are nanoparticles comprising compounds (e.g., JAK2-, ADAR1- and BCR-ABL1-inhibitory small molecules, nucleic acids and polypeptides) used to practice methods as provided herein in the form of active agent-containing nanoparticles (e.g., a secondary nanoparticle), as described, e.g., in U.S. Pat. Pub. No. 20070077286. In one embodiment, provided are nanoparticles comprising a fat-soluble active agent used to practice a method as provided herein or a fat-solubilized water-soluble active agent to act with a bivalent or trivalent metal salt.

In one embodiment, solid lipid suspensions can be used to formulate and to deliver formulations, pharmaceutical compositions and compounds used to practice methods as provided herein to mammalian cells in vivo, in vitro or ex vivo, as described, e.g., in U.S. Pat. Pub. No. 20050136121.

### Delivery Vehicles

In alternative embodiments, any delivery vehicle can be used to practice the methods as provided herein, e.g., to deliver compositions methods as provided herein (e.g., e.g., JAK2-, ADAR1- and BCR-ABL1-inhibitory small molecules, nucleic acids and polypeptides) to mammalian cells in vivo, in vitro or ex vivo. For example, delivery vehicles comprising polycations, cationic polymers and/or cationic peptides, such as polyethyleneimine derivatives, can be used e.g. as described, e.g., in U.S. Pat. Pub. No. 20060083737.

In one embodiment, a dried polypeptide-surfactant complex is used to formulate a composition used to practice a method as provided herein, e.g. as described, e.g., in U.S. Pat. Pub. No. 20040151766.

In one embodiment, a composition used to practice methods as provided herein can be applied to cells using vehicles with cell membrane-permeant peptide conjugates, e.g., as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, e.g., as described in U.S. Pat. No. 5,846,743, describing transport-mediating peptides that are highly basic and bind to poly-phosphoinositides.

In one embodiment, electro-permeabilization is used as a primary or adjunctive means to deliver the composition to a cell, e.g., using any electroporation system as described e.g. in U.S. Pat. Nos. 7,109,034; 6,261,815; 5,874,268.

### Products of Manufacture and Kits

Provided are products of manufacture and kits for practicing methods as provided herein, and in alternative embodiments, the kits also comprise instructions for practicing methods as provided herein.

In alternative embodiments, products of manufacture as provide herein, e.g., implants, particles, a nanoparticle, a particle, a micelle or a liposome or lipoplex, a polymersome, a polyplex or a dendrimer, comprise formulations, pharmaceutical compositions and compounds used to practice methods as provided herein, e.g., comprising JAK2-, ADAR1- and BCR-ABL1-inhibitory small molecules, nucleic acids and polypeptides.

### Crosslinking Immunoprecipitation (CLIP)

In alternative embodiments, provided herein is a method of detecting edited and unedited RNA transcripts binding to ADAR1 protein using Crosslinking Immunoprecipitation (CLIP) or RNA immunoprecipitation (RIP) or equivalents with an ADAR1 antibody or ADAR1 binding protein.

In alternative embodiments, CLIP methods that can be used include: high-throughput sequencing-CLIP (HITS-CLIP), Photoactivatable-Ribonucleoside Enhanced CLIP (PAR-CLIP), and Individual CLIP (iCLIP) and CLIP (e.g., iCLIP) protocols can include e.g., those described in Konig et al. J. Vis. Exp. 2011.

In alternative embodiments, methods provided herein provide improved detection since edited mRNA and microRNA transcripts are subject to degradation and sequestration, and current detection methods fail to identify them. Methods as provided herein using e.g., CLIP assays with RNA-sequencing, can detect and identify more transcripts binding to ADARs, and provide potential new therapeutic targets and biomarkers that can be used to determine disease progression and therapeutic efficacy.

In alternative embodiments, CLIP comprises use of in vivo cross-linking of RNA-protein complexes using ultraviolet light (UV), or equivalents. UV radiation causes covalent bonds to be formed between the ADAR1 and the RNA to which it is bound. The cross-linked cells are then lysed, and ADAR1 is isolated via immunoprecipitation. In alternative embodiments, to facilitate sequence specific priming of reverse transcription, RNA adapters are ligated to the 3′ ends of the RNA. Radiolabeled phosphates can be transferred to the 5′ ends of the RNA fragments. The RNA-protein complexes are then separated from free RNA using chromatography, gel electrophoresis, membrane transfer or equivalents. Digestion is then performed to remove protein from the RNA-protein complexes (e.g., using Proteinase K). This step leaves a peptide at the cross-link site, allowing for the identification of the cross-linked nucleotide. After ligating RNA linkers to the RNA 5′ ends, cDNA is synthesized via RT-PCR. Sequencing, e.g., high-throughput sequencing, can be used to generate reads containing distinct barcodes that identify the last cDNA nucleotide. Interaction sites can be identified by mapping the reads back to the transcriptome.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (1989) Molecular Cloning: A Laboratory Manual, Second Edition, Cold Spring Harbor Laboratory Press, N.Y. and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, N.Y., Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1

### Methods for Inhibiting Leukemia Stem Cell (LSC) Generation

This example demonstrates that methods and compositions as provided herein, including pharmaceutical compositions and formulations, products of manufacture and kits, and methods of using them, can be effective for treating or preventing leukemias by e.g., antagonizing ADAR1's effect on LSC self-renewal, and inhibiting let-7 pri microRNA editing and LIN28B upregulation.

Post-transcriptional adenosine-to-inosine RNA editing mediated by double-stranded RNA-specific adenosine deaminase (also called Adenosine Deaminase Acting on RNA1, or ADAR1) promotes cancer progression and therapeutic resistance. However, ADAR1 editase-dependent mechanisms governing leukemia stem cell (LSC) generation have not been elucidated.

Here, in blast crisis chronic myeloid leukemia (BC CML) we show that increased Janus kinase 2 (JAK2) signaling and BCR-ABL1 amplification converge on ADAR1 activation. Selective JAK2 and BCR-ABL1 inhibition prevents LSC self-renewal in a humanized BC CML mouse model commensurate with ADAR1 downregulation. Lentiviral ADAR1, but not an editing defective ADAR1^{E912} mutant, induces self-renewal gene expression and impairs biogenesis of stem cell regulatory let-7 microRNAs. Combined RNA sequencing, qRT-PCR, CLIP-ADAR1, and pri-let-7 mutagenesis data suggest that ADAR1 promotes LSC generation via let-7 pri microRNA editing and LIN28B upregulation. A small molecule tool compound antagonizes ADAR1′s effect on LSC self-renewal in stromal co-cultures and restores let-7 biogenesis. Thus, ADAR1 editase activation represents a unique therapeutic vulnerability in LSC with active JAK2 signaling.

### JAK2 Signaling Increases ADAR1 Expression in Progenitor LSCs

Previous studies suggest that ADAR1 expression is enhanced by inflammatory cytokine signaling that activates STAT binding to the ADAR1 promoter (George et al., 2008; George and Samuel, 2015). Thus, cytokine receptor and downstream target gene expression were analyzed by RNA-seq of fluorescence-activated cell sorting (FACS)-purified normal, CP, and BC progenitors (Table S1, as illustrated in FIG. 22A and 22B).

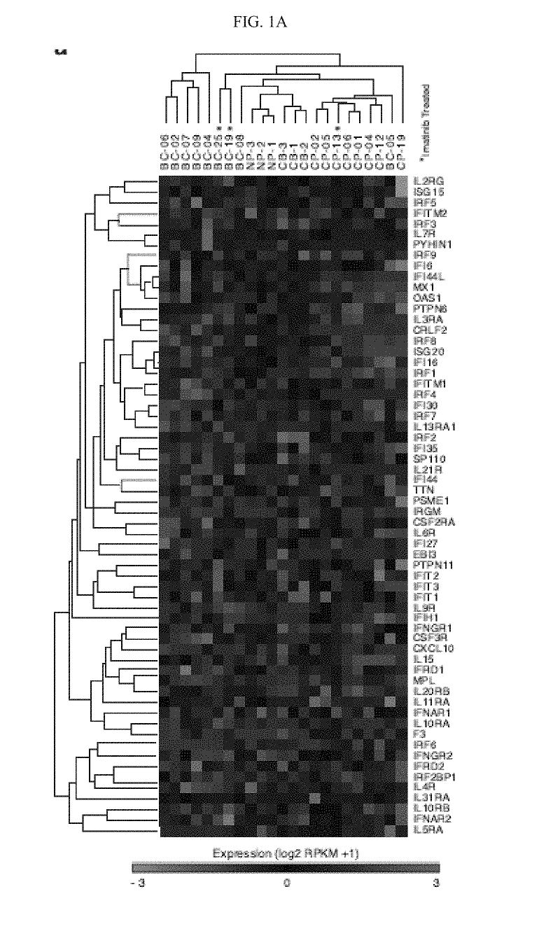

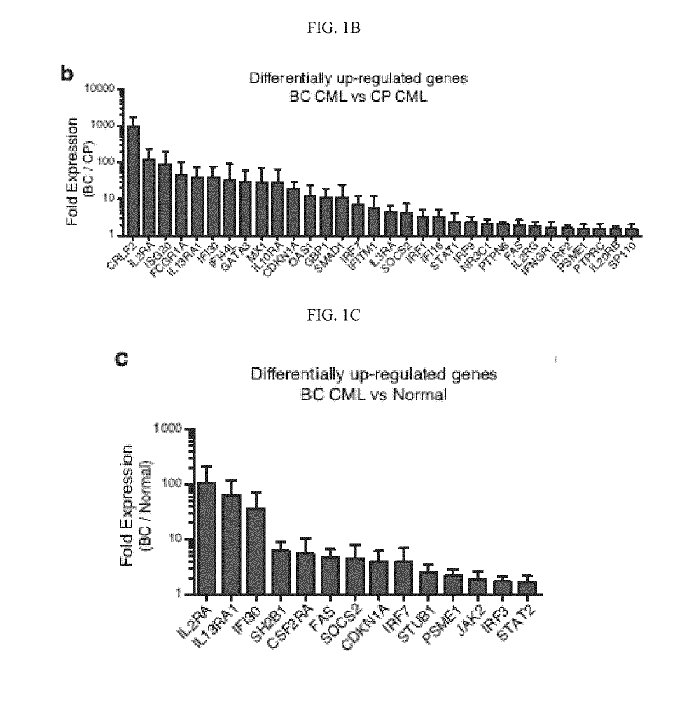

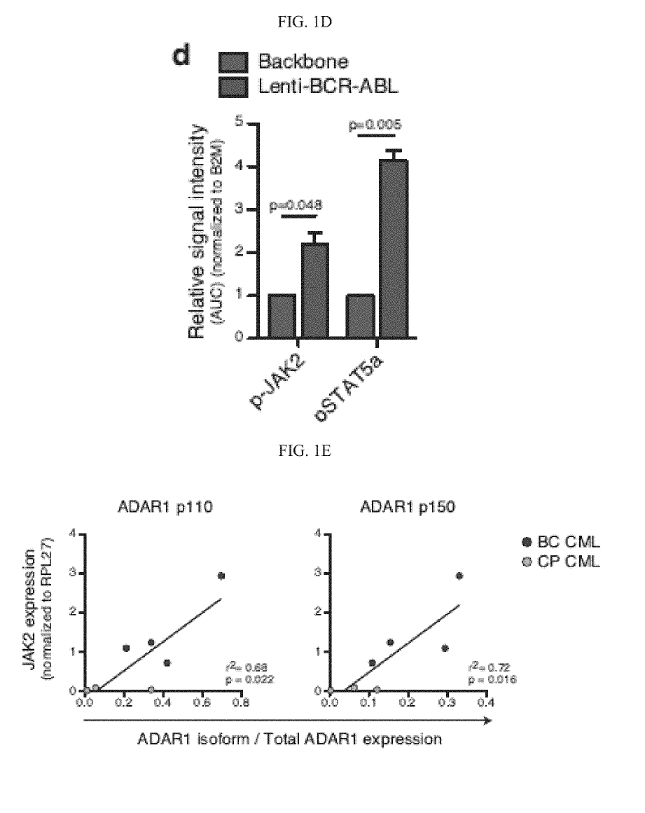

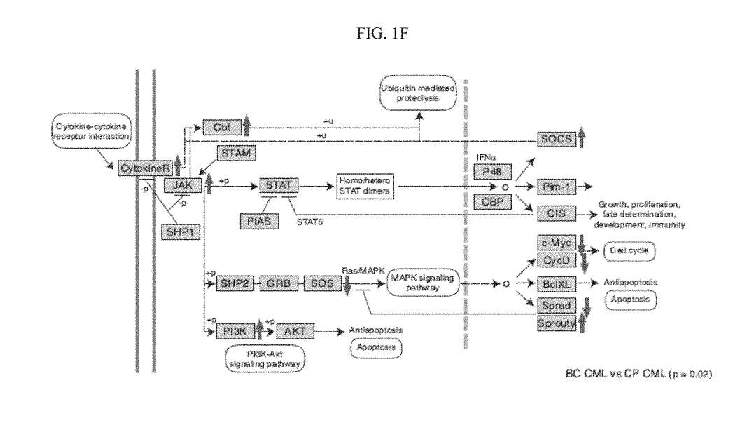

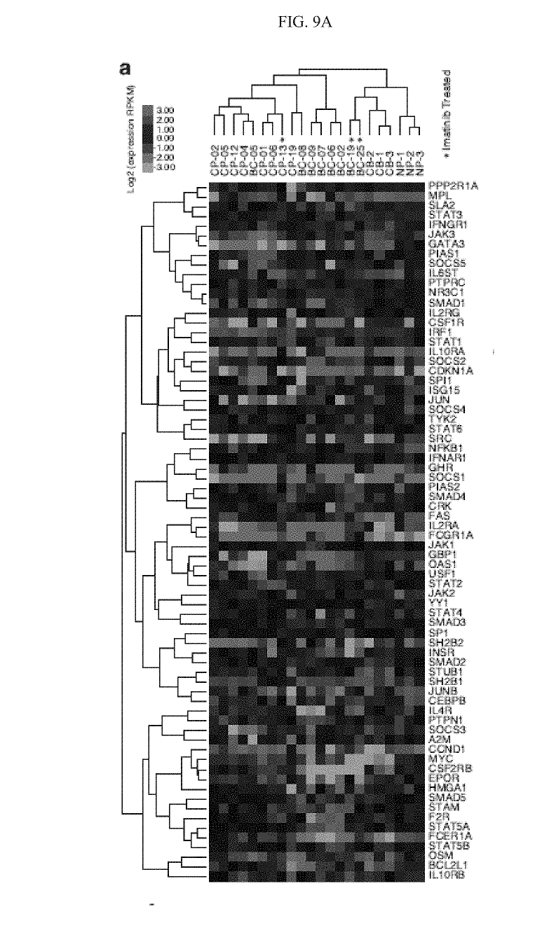

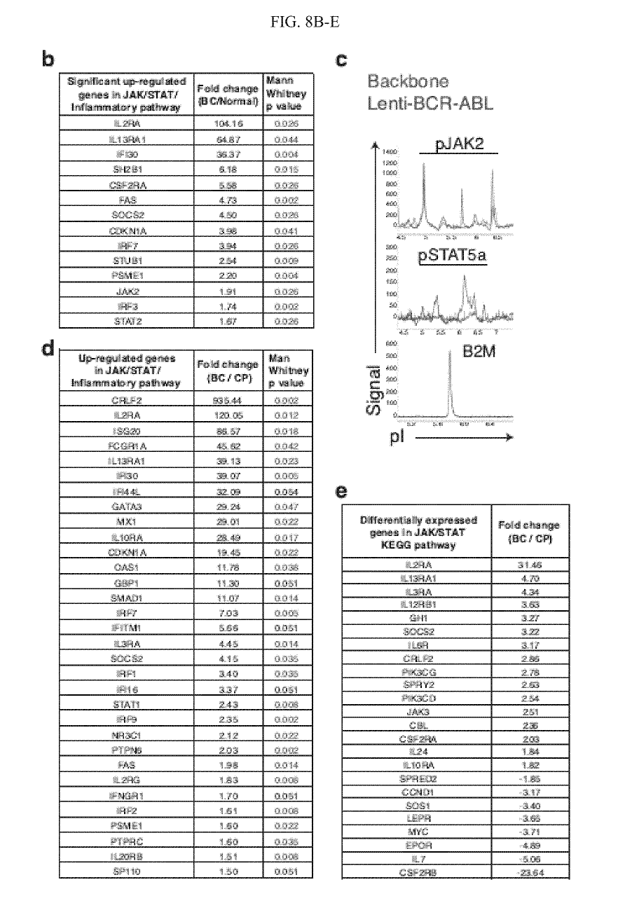

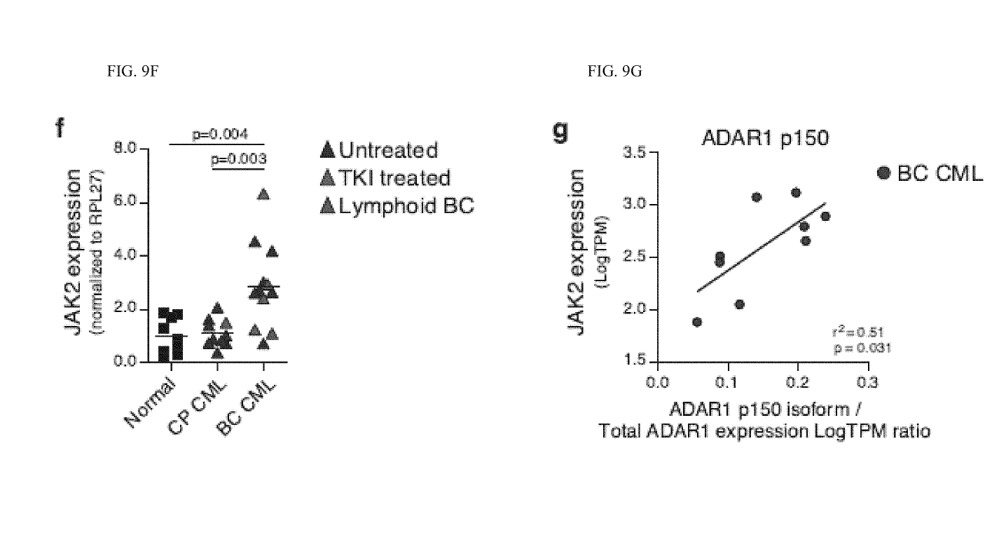

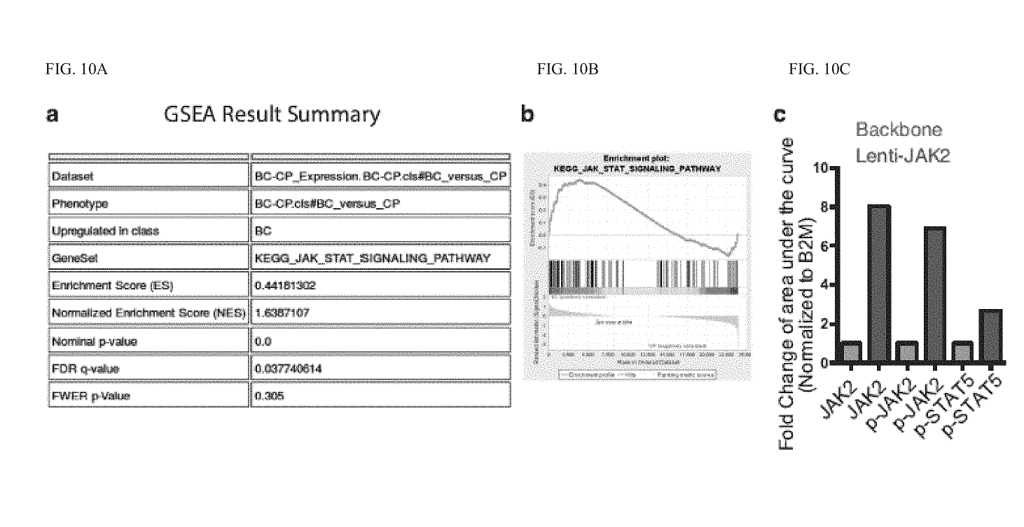

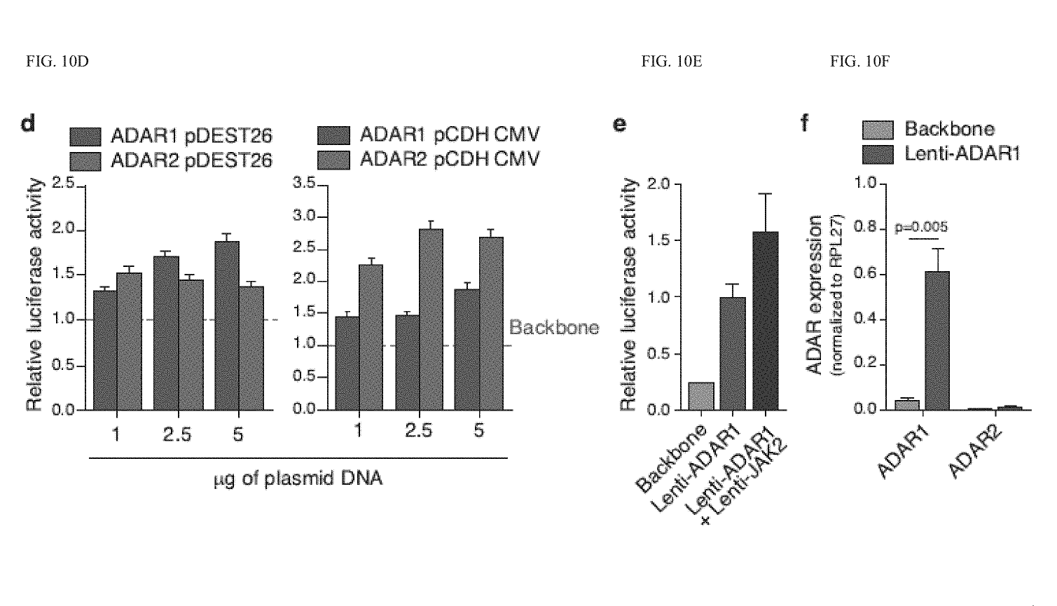

Inflammatory cytokine-associated receptors, which signal through the JAK-STAT pathway, and their downstream target genes were increased in BC compared with normal and CP progenitors (FIGS. 1A and 9A-E). Compared with CP, BC progenitors harbored increased levels of IFNγR1 and IL-3Rα, which can signal through JAK2 and activate ADAR1 transcription by binding of STATs to the ADAR1 promoter (FIG. 1B) (George and Samuel, 2015). In addition, BC CML showed increased expression of JAK/STAT signaling pathway genes and JAK2 transcripts compared with normal progenitors (FIGS. 1C and 9F). Nanoproteomics analysis of protein expression demonstrated elevated p-STAT5a levels following lentiviral BCR-ABL1 amplification (FIGS. 1D and 9C). Inflammatory cytokine responsive ADAR1 p150 isoform expression correlated with JAK2 expression in both qRT-PCR and RNA-seq analysis of BC progenitors (FIGS. 1E and 9G). Moreover, KEGG pathway-based gene set enrichment analysis (GSEA) involving 1495 genes at a FDR of 0.05, revealed that the JAK/STAT signaling pathway was one of the most significantly differentially expressed gene sets (p=0.02, Benjamini adjusted-p value) in BC compared with CP progenitors (FIGS. 1F and 10A-B). These data highlight the increased sensitivity of BC progenitors to JAK/STAT signaling, which could activate ADAR1-mediated RNA editing in inflammatory microenvironments.

### JAK2 Signaling Promotes ADAR1-mediated A-to-I Editing

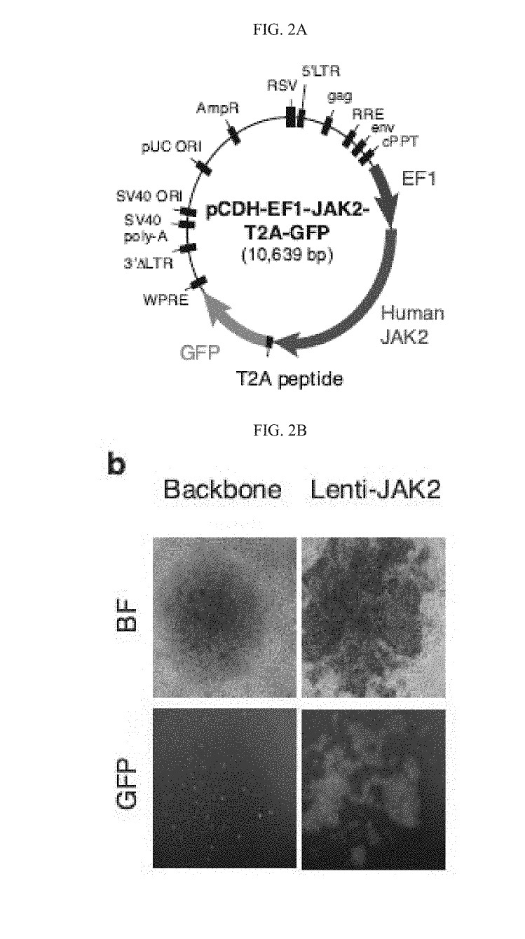

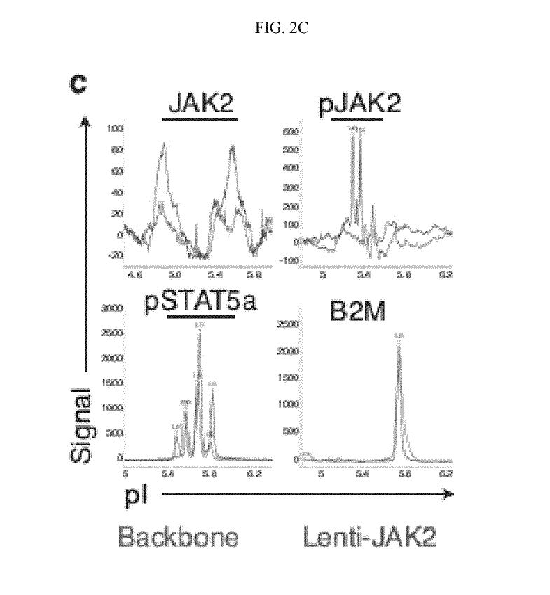

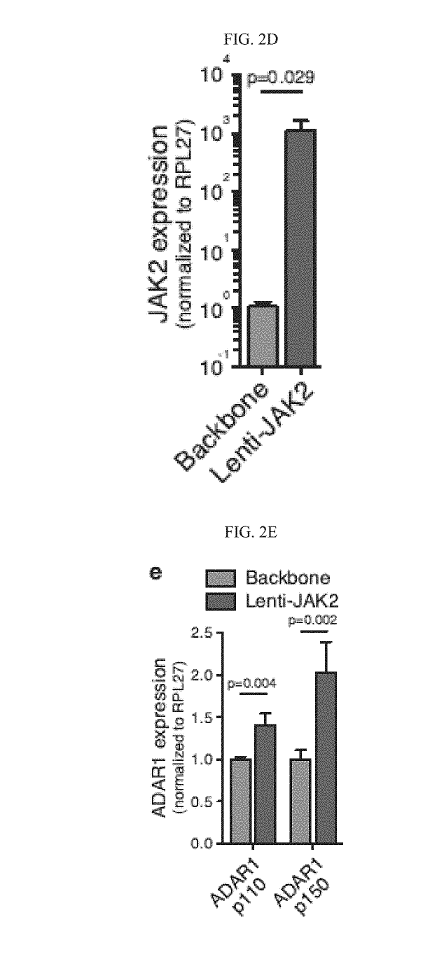

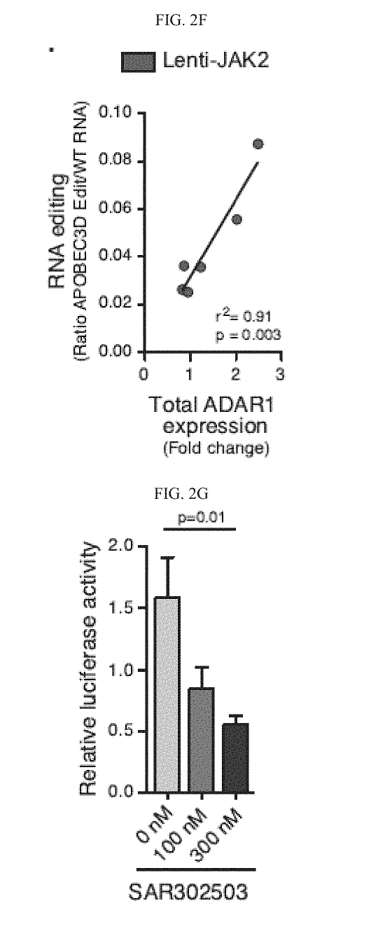

To investigate the direct contribution of JAK2 signaling to activation of ADAR1-mediated adenosine-to-inosine (A-to-I) editing, we developed a lentiviral construct that enabled robust expression of human JAK2-GFP in normal CD34⁺ progenitors (FIG. 2A). Transduction efficiency was confirmed by fluorescence microscopy, nanoproteomics analysis of p-JAK2 and pSTAT5a and JAK2 qRT-PCR (FIGS. 2B-D and 10C). Lentiviral JAK2 transduction of human CD34+ progenitors enhanced both ADAR1 p150 and p110 transcript expression and ADAR1's A-to-I editing activity as measured by RNA Editing Site-Specific qPCR (RESSqPCR) (FIGS. 2E-F) (Crews et al., 2015). While lentiviral ADAR1 transduction of BCR-ABL1+K562 leukemia cells potentiated A-to-I RNA editing as measured by luciferase reporter activity in both the pCDH lentiviral vector and pDEST26 mammalian expression vector, this activity was further enhanced by addition of JAK2 expression (FIGS. 10D-F). Selective JAK2 inhibition with a pharmacological inhibitor (SAR302503) (Geron et al., 2008) reversed RNA editing activity in a dose dependent manner in K562 cells (FIG. 2G). Taken together, these data suggest that JAK2 signaling enhances ADAR1-mediated RNA editing.

### JAK2 and BCR-ABL1 Regulate Let-7 Biogenesis and LSC Self-Renewal

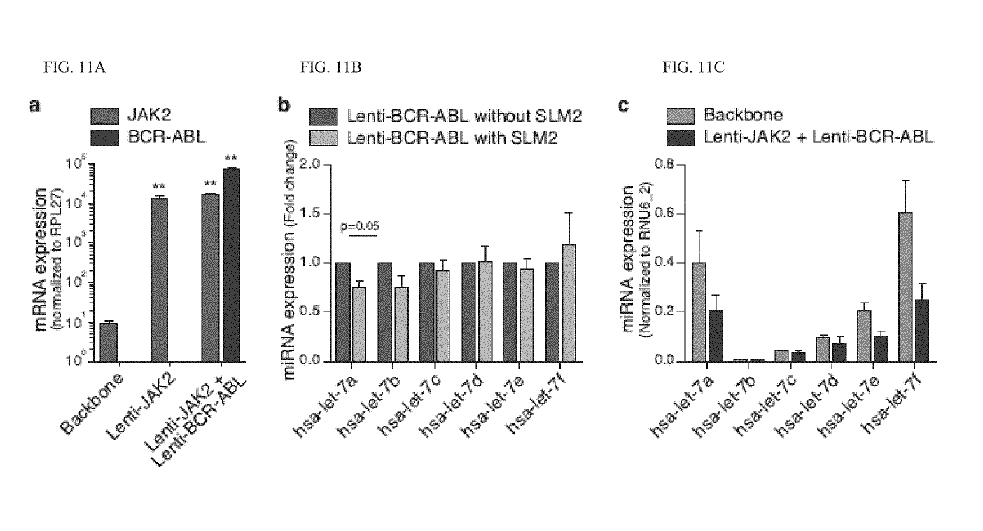

While ADAR1 editing targets have not been completely elucidated, emerging data suggest that ADAR1 impairs biogenesis of tumor suppressive miRNAs, thereby contributing to cancer progression (Mariner et al., 2008). Because the JAK2/STAT pathway activated ADAR1, it was hypothesized that the LIN28B/let-7 self-renewal axis may also be disrupted by increased JAK2 signaling. Indeed, qRT-PCR assays demonstrated that efficient JAK2 transduction increased LIN28B pluripotency gene expression and inhibited the expression of let-7 family miRNAs (FIGS. 2H-I). Amplification of BCR-ABL1 is a hallmark of BC transformation and has been linked to LIN28B upregulation (Viswanathan et al., 2009). RNA-seq analysis was performed of lentiviral BCR-ABL1 transduced normal progenitors. Notably, a BCR-ABL1 transduction efficiency dependent increase in LIN28B expression was observed and was potentiated by co-transduction with JAK2 (FIGS. 2J-K and 10G). In human LSC-supportive SL/M2 stromal co-cultures, let-7a expression was significantly reduced in BCR-ABL1 transduced progenitors (FIG. 10H). However, biogenesis of let-7 family members was more profoundly impaired following co-transduction with JAK2 and BCR-ABL1 (FIG. 10I). Treatment of JAK2/BCR-ABL1 transduced normal CD34⁺ progenitors with the JAK2 inhibitor SAR302503 showed reduced ADAR1 p150 isoform expression, coupled with restored let-7 miRNA biogenesis in a dose-dependent manner (FIGS. 11A-B). These results confirmed the hypothesis that the JAK2/ADAR1 axis promotes maintenance of self-renewal commensurate with impaired let-7 miRNA biogenesis. Moreover, RNA-seq analysis revealed decreased expression of pri-miRNA processing genes, such as DGCR8 and ILF3, in BC compared with CP progenitors and in normal CD34⁺ cord blood cells transduced with ADAR1 WT compared with backbone controls; see Tables S2 and S3.

Tables S2a-b are summary tables of genes involved in microRNA biogenesis from RNA-seq-based gene expression analysis of BC (n=6) compared with CP (n=7) CML untreated patient samples Tables S2(a) or in BC CIVIL (n=6) compared with normal PB (n=3) untreated patient samples Tables S2(b); p values were calculated using Student's t-test.

(a)

| | Fold change | |
|---|---|---|
| Gene | (BC/CP) | p value |
| ENSG00000108654_DDX5 | 2.01 | 0.038 |
| ENSG00000143621_ILF2 | 0.96 | 0.684 |
| ENSG00000198646_NCOA6 | 0.84 | 0.275 |
| ENSG00000100697_DICER 1 | 0.83 | 0.351 |
| ENSG00000129351_ILF3 | 0.76 | 0.029 |
| ENSG00000197157_SND 1 | 0.73 | 0.132 |
| ENSG00000128191_DGCR8 | 0.71 | 0.036 |
| ENSG00000113360_DR0SHA | 0.70 | 0.120 |
| ENSG00000100201_DDX17 | 0.67 | 0.332 |
| ENSG00000235706_DICER1_AS 1 | 0.64 | 0.415 |
| ENSG00000124571_XP05 | 0.53 | 0.058 |

(b)

| | Fold change | |
|---|---|---|
| Gene | (BC/NP) | p value |
| ENSG00000108654_DDX5 | 1.73 | 0.215 |
| ENSG00000100201_DDX17 | 1.49 | 0.177 |
| ENSG00000129351_ILF3 | 1.42 | 0.101 |
| ENSG00000100697_DICER 1 | 1.39 | 0.167 |
| ENSG00000113360_DR0SHA | 1.30 | 0.307 |
| ENSG00000198646_NC0A6 | 1.24 | 0.071 |
| ENSG00000128191_DGCR8 | 1.12 | 0.511 |
| ENSG00000143621_ILF2 | 0.69 | 0.016 |
| ENSG00000124571_XP05 | 0.55 | 0.067 |
| ENSG00000197157_SND 1 | 0.54 | 0.013 |
| ENSG00000235706_DICER1_AS 1 | 0.53 | 0.054 |

Tables S3a-b are summary tables of genes involved in microRNA biogenesis from RNA-seq-based gene expression analysis of normal CD34+ cord blood cells: transduced with lenti-ADAR1 WT compared with backbone control (n=3 each) Tables S2(a), or lenti-ADAR1 Mutant compared to backbone control (n=3 each) Tables S2(b). p values were calculated using Student's t-test.

| | Fold change | |
|---|---|---|
| Gene | (WT/pCDH) | p value |
| ENSG00000100201_DDX17 | 3.88 | 0.033 |
| ENSG00000197157_SND 1 | 1.46 | 0.069 |
| ENSG00000108654_DDX5 | 1.08 | 0.313 |
| ENSG00000113360_DR0SHA | 0.99 | 0.887 |
| ENSG00000100697_DICER 1 | 0.92 | 0.683 |
| ENSG00000198646_NC0A6 | 0.91 | 0.441 |
| ENSG00000143621_ILF2 | 0.90 | 0.268 |
| ENSG00000124571_XP05 | 0.88 | 0.454 |
| ENSG00000128191_DGCR8 | 0.88 | 0.304 |
| ENSG00000129351_ILF3 | 0.86 | 0.171 |
| ENSG00000235706_DICER 1-AS1 | 0.84 | 0.174 |

(b)

| | Fold change | |
|---|---|---|
| Gene | (Mut/pCDH) | p value |
| ENSG00000235706_DICER 1-AS1 | 2.25 | 0.031 |
| ENSG00000129351_ILF3 | 2.25 | 0.034 |
| ENSG00000128191_DGCR8 | 1.21 | 0.024 |
| ENSG00000124571_XP05 | 1.03 | 0.848 |
| ENSG00000113360_DR0SHA | 0.94 | 0.370 |
| ENSG00000143621_ILF2 | 0.91 | 0.742 |
| ENSG00000100697_DICER 1 | 0.88 | 0.564 |
| ENSG00000197157_SND 1 | 0.73 | 0.163 |
| ENSG00000108654_DDX5 | 0.71 | 0.163 |
| ENSG00000198646_NC0A6 | 0.68 | 0.040 |
| ENSG00000100201_DDX17 | 0.59 | 0.047 |

These results revealed a LSC intrinsic defect in let-7 miRNA maturation. In keeping with disruption of the LIN28B/let-7 self-renewal axis, combined JAK2 and BCR-ABL1 transduction enhanced colony-replating capacity as an in vitro surrogate measure of self-renewal (FIG. 2L-M). Conversely, combined inhibition of JAK2 with SAR302503 (Geron et al., 2008) and BCR-ABL1 with dasatinib significantly inhibited their self-renewal potential (FIG. 2N). Together these data suggested that JAK2 and BCR-ABL1 enhanced malignant reprogramming of progenitors into LSCs by deregulating LIN28B expression and impairing let-7 miRNA biogenesis.

### JAK2 and BCR-ABL1 Inhibition Prevents Self-Renewal of ADAR1-Expressing LSCs

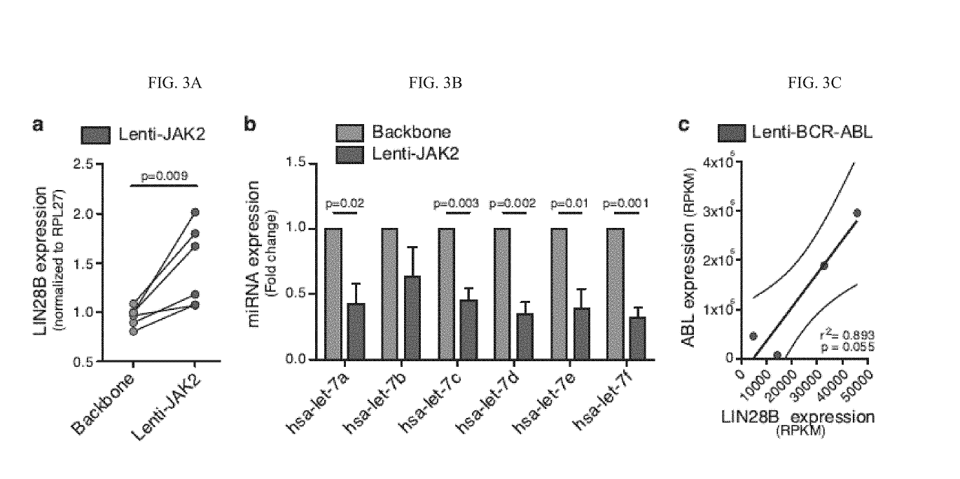

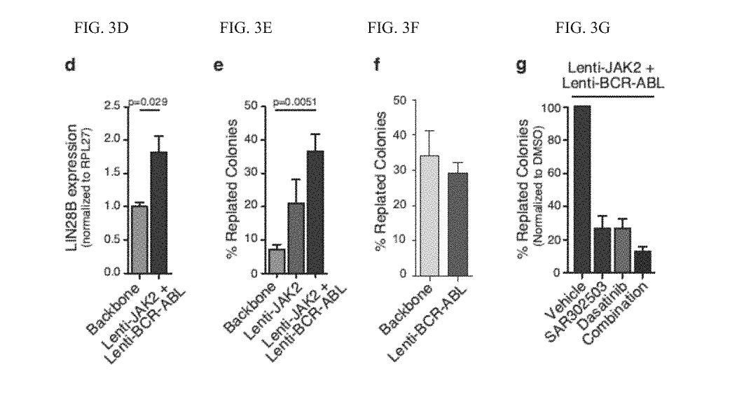

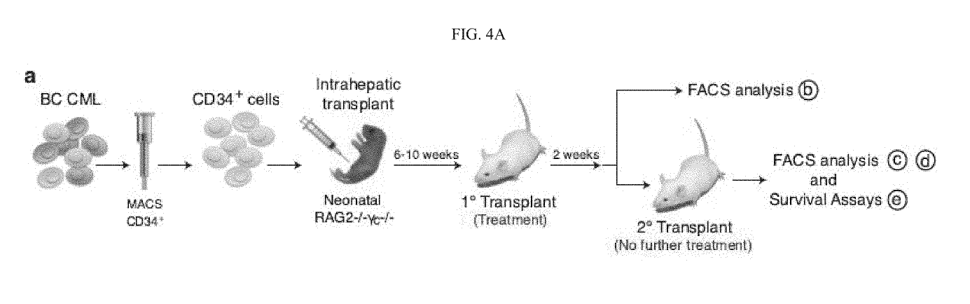

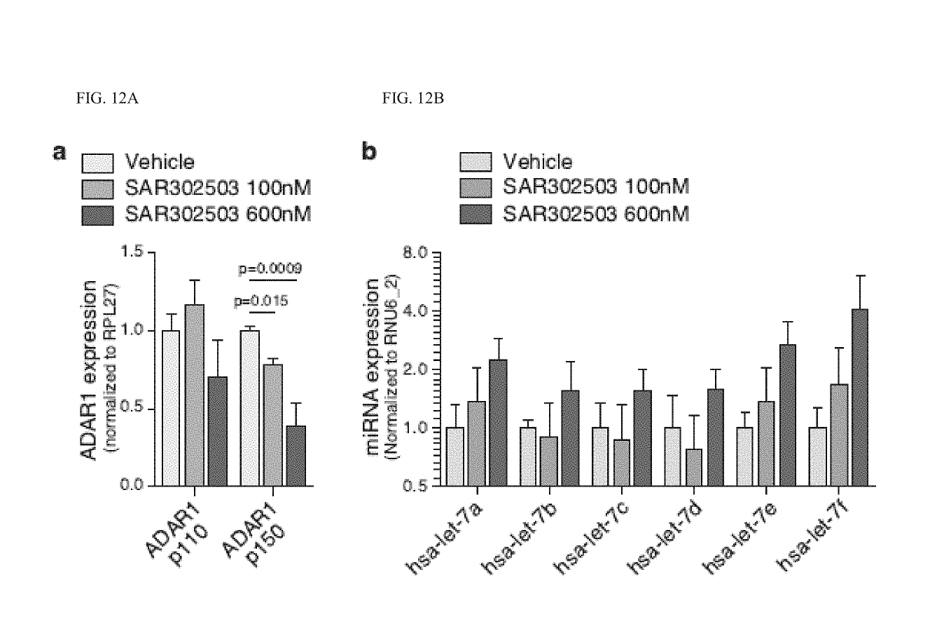

To understand the convergence of JAK2 and ADAR1 pathways on BCR-ABL1⁺ BC CML LSC maintenance, RAG2^{-/-}γ^{-/-} mice engrafted with human BC CD34⁺ cells were treated with SAR302503, dasatinib or the combination (FIG. 3A). Primary human leukemic engraftment was observed in all hematopoietic tissues and the progenitor cell population was enriched for granulocyte-macrophage progenitors (GMP) that harbored enhanced serial transplantation potential (Abrahamsson et al., 2009; Jamieson et al., 2004) (FIGS. 12A and B).

After two weeks of treatment, FACS analysis of hematopoietic tissues showed that both dasatinib and combination treatment inhibited BC LSC survival compared to vehicle-treated controls (FIGS. 3B and 12C-E). Selective JAK2 inhibition by SAR302503 in the splenic and bone marrow niches was confirmed by nanoproteomic analysis of p-JAK2, p-CRKL and p-STAT5a levels in BC progenitors isolated from treated mice (FIG. 12F). However, only combination treatment significantly reduced self-renewal of BCR-ABL1 expressing BC progenitors, particularly leukemic GMP, in the bone marrow thereby prolonging survival of serially transplanted mice (FIGS. 3C-F). A significant reduction in ADAR1 p150 transcripts was also observed following combination treatment, suggesting that JAK2 and BCR-ABL1 signaling converge on ADAR1 to enhance LSC propagation (FIG. 3G).

### Inhibition of ADAR1-Mediated RNA Editing Impairs Self-Renewal

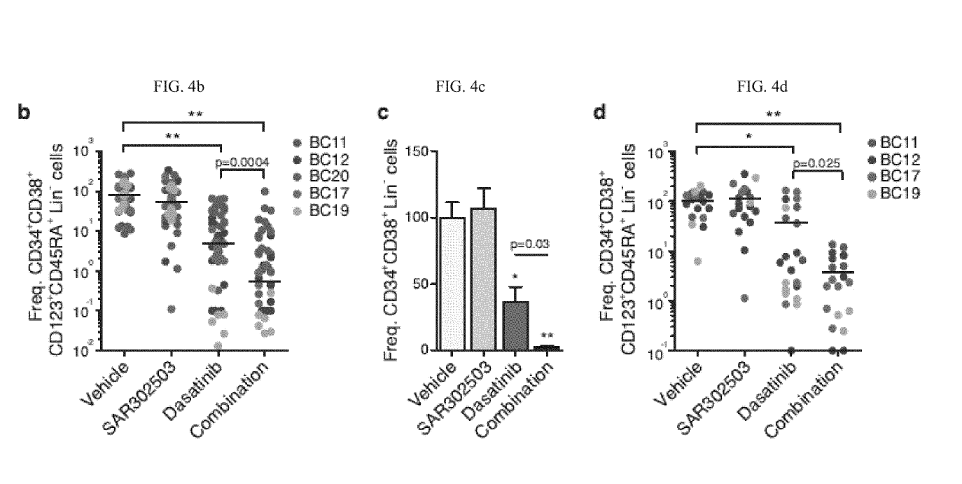

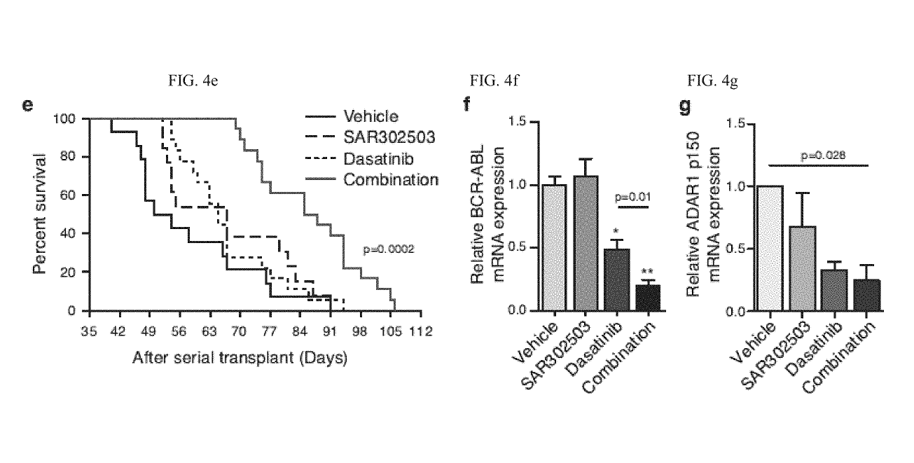

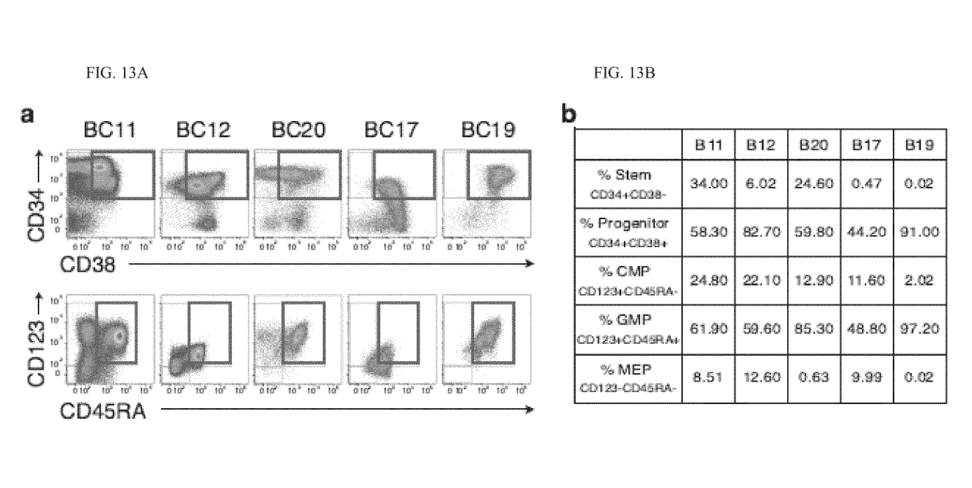

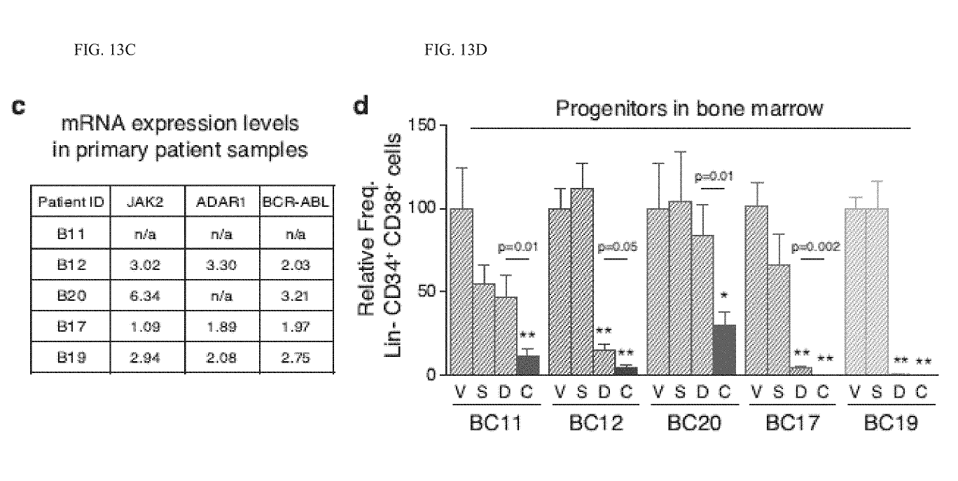

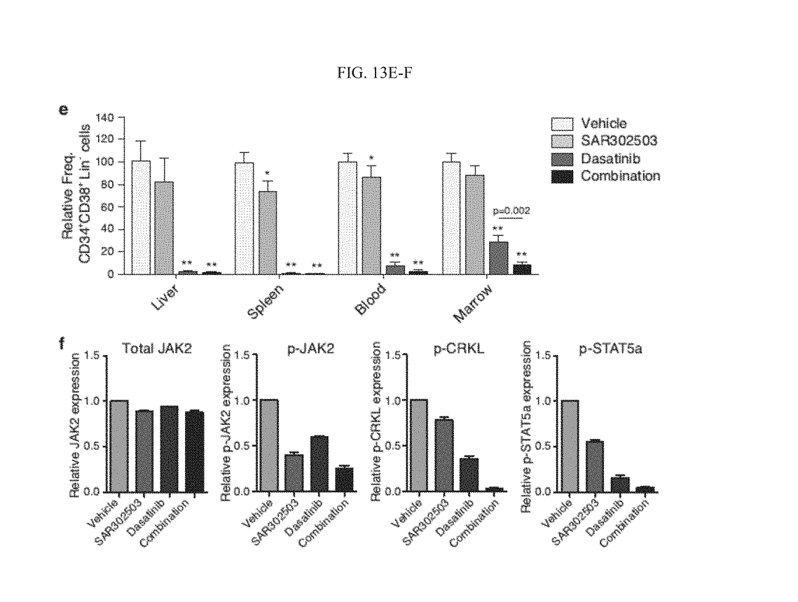

By lentivirally expressing ADAR1 p150 wild-type (ADAR1 WT) or an editing defective mutant vector ADAR1 Mut (ADAR1^{E912A}) it was established that an inhibitory tool compound, 8-azadenosine (8-aza) (Veliz et al., 2003) reduced ADAR1's adenosine-to-inosine (A-to-I) editing activity in K562 leukemic cell line (FIG. 4A-B). Treatment with 8-aza showed no effect on BCR-ABL and JAK2 signaling, as demonstrated by qRT-PCR analysis and p-CRKL and p-STAT5a Western blot analysis (FIG. 13A-B). To determine if ADAR1 is required for LSC self-renewal, BC CML, normal or lentiviral JAK2 and BCR-ABL1 transduced CD34⁺ progenitors were plated in SL/M2 stromal cultures and treated with 8-aza, SAR302503, or the combination. After two weeks in stromal co-culture, SAR302503 and 8-aza restored let-7 miRNA biogenesis commensurate with a reduction in ADAR1 expression, RNA editing activity and LIN28B expression in JAK2/BCR-ABL1 transduced progenitors compared with controls (FIGS. 4C-G). Moreover, replating capacity of primary CML BC progenitors was significantly reduced by combined JAK2 inhibitor and 8-aza treatment in stromal co-cultures (FIGS. 4H-I). Since normal CD34⁺ hematopoietic progenitors harbored relatively low JAK2 and ADAR1 levels, their self renewal capacity was not significantly impaired by 8-aza or combination treatment (FIGS. 4H-I) indicative of an adequate therapeutic index between normal and malignant progenitors.

### ADAR1 Enhances Self-Renewal Gene Expression

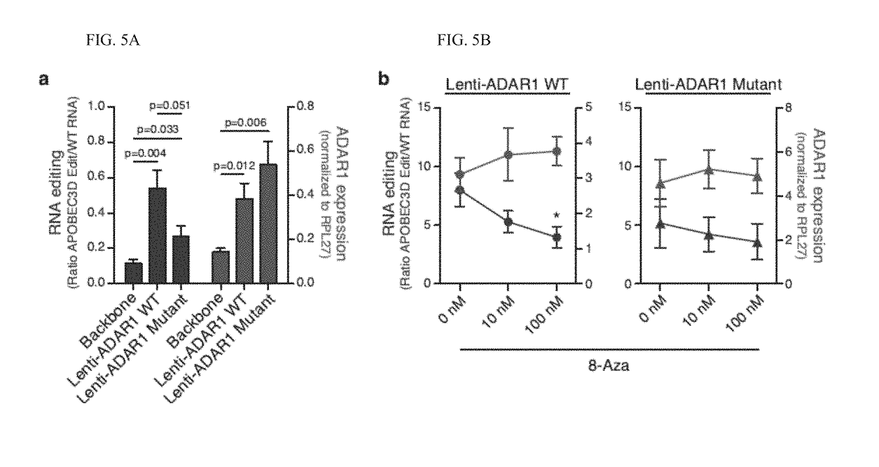

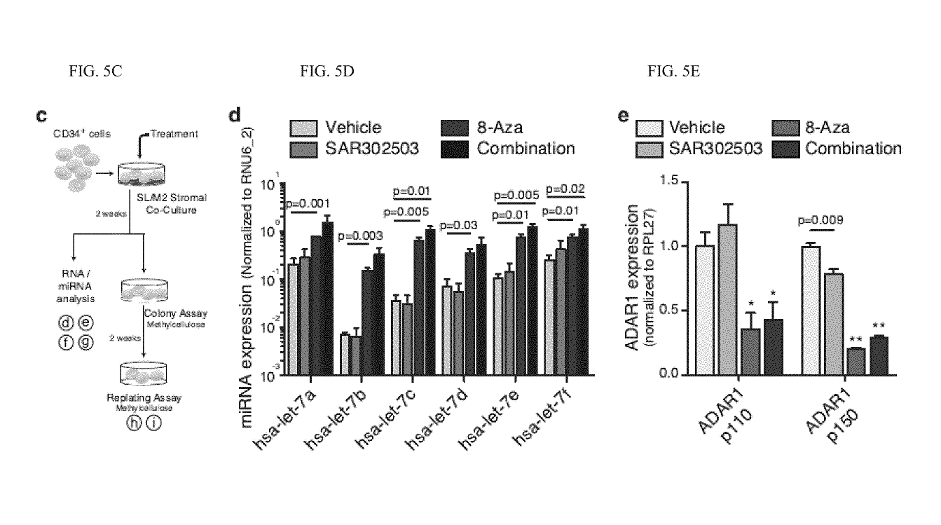

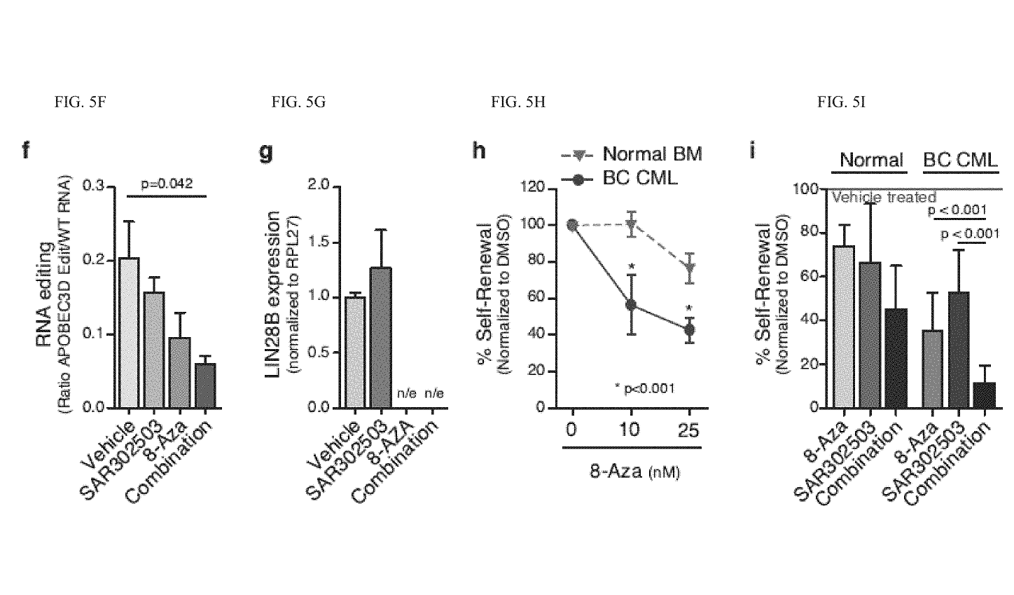

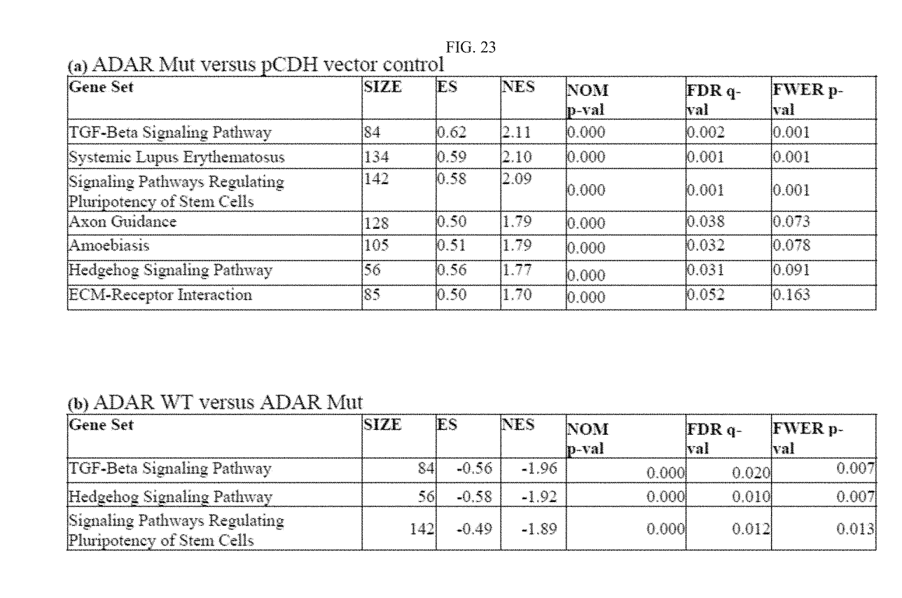

To understand the mechanisms governing ADAR1-mediated LSC self-renewal, RNA-seq analysis was performed of cord blood CD34⁺ cells lentivirally transduced with lentiviral backbone vector, ADAR1, or an adenosine-to-inosine (A-to-I) editing defective ADAR1^{E912A} mutant vector. A KEGG pathway-based gene set enrichment analysis (GSEA) revealed that ADAR1 overexpression significantly affected genes involved in the regulation of stem cell pluripotency, see FIGS. 5A-B and FIG. 23, which illustrates Table S4; Tables S4a-b data shows significantly affected KEGG pathway genes in normal cord blood CD34+ progenitors transduced with Tables S(a) lenti-ADAR1 Mut (n=3) compared to vector controls (n=3), and Tables S(b) lenti-ADAR1 WT (n=3) compared to lenti-ADAR1 Mut (n=3), analyzed by RNA-seq and Gene Set Enrichment Analysis (GSEA).

Transcriptomic analysis of ADAR1 compared with backbone-transduced CD34⁺ cells revealed fourteen upregulated and five downregulated stem cell pluripotency regulatory genes (FIG. 5C). Interestingly, the most differentially upregulated genes, including ACVR2B, HRAS, and FGFR3, were let-7 targets, suggesting that ADAR1-mediated impairment of let-7 miRNA biogenesis may also regulate LSC self-renewal capacity (FIG. 5D-E). Lentivirally enforced let-7a and let-7d expression reduced progenitor total colony numbers. Colony replating capacity and LIN28B expression decreased thereby underscoring the inhibitory impact of let-7 on self-renewal (FIGS. 5F-K and 13C-D).

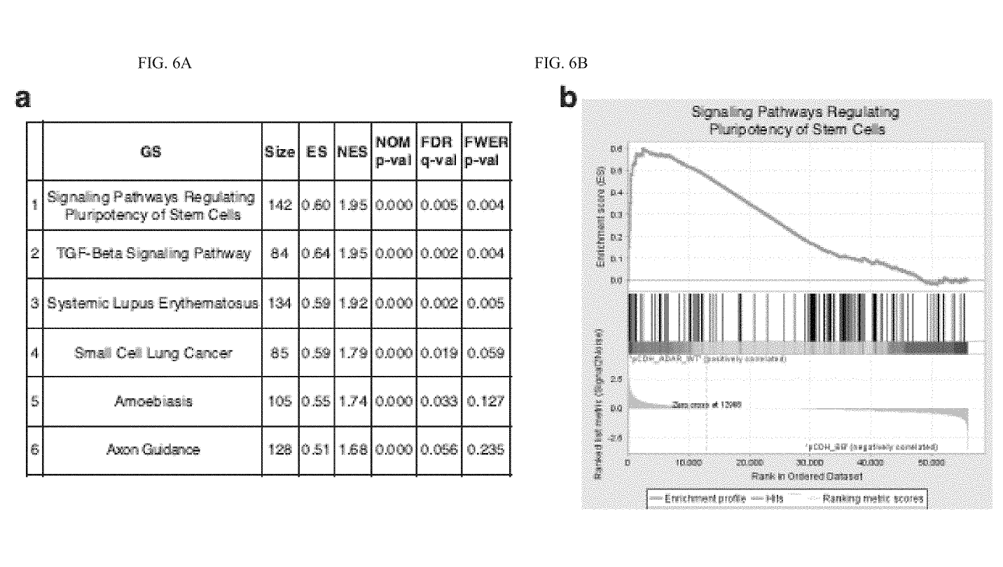

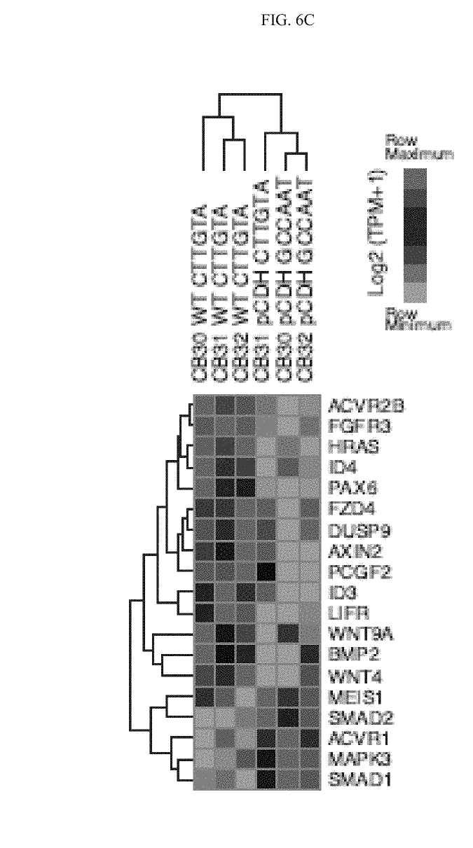

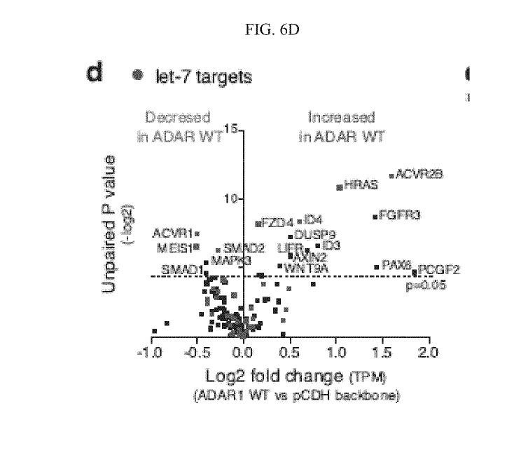

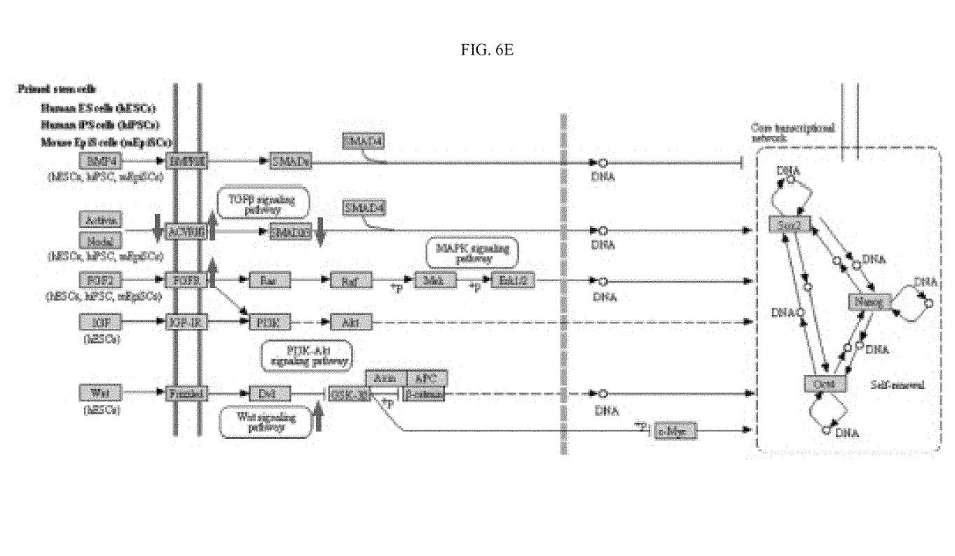

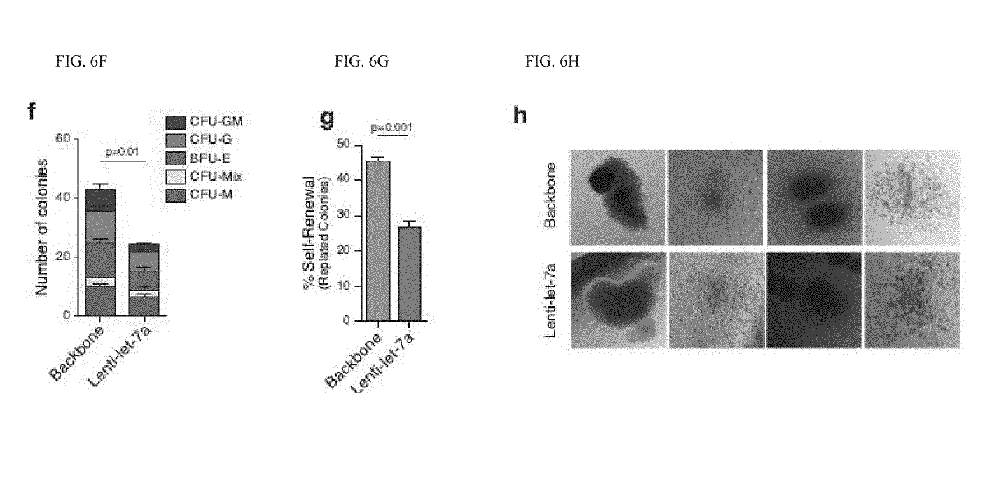

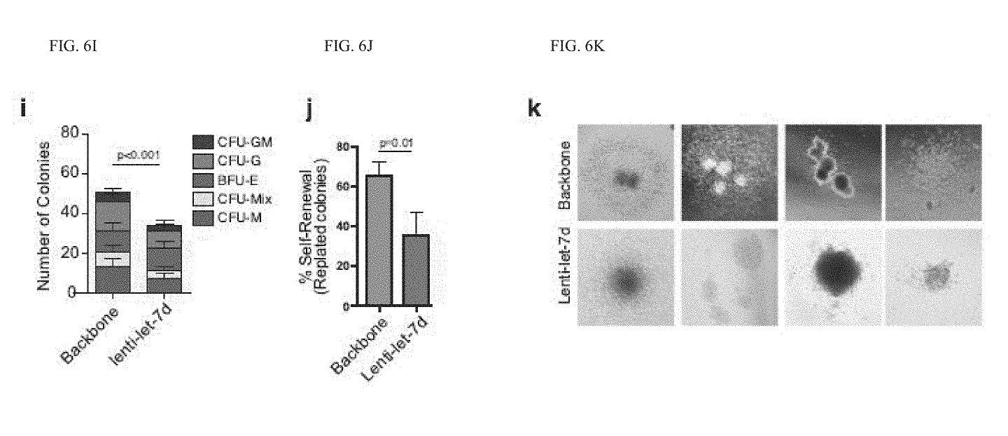

Since JAK2 activates ADAR1-mediated RNA editing and impairs let-7 biogenesis (FIGS. 2E-F and I), it was decided to investigate if let-7 biogenesis in LSC is restricted by cytokine-responsive ADAR1 editing activity. First, we observed a reduction in pri- and pre-let-7a-1, let-7a-2, let?-a-3, pri-let-7d expression using RNA-seq quantification, as well as decreased expression of mature let-7 miRNAs in BC compared to CP progenitors (FIG. 6A-B). Lentivirally enforced ADAR1 expression increased replating potential of CP CML coupled with a reduction in mature let-7d expression, suggesting malignant activation of ADAR1 in CP to BC transition is coupled with impaired let-7 biogenesis and enhanced LSC self-renewal (FIGS. 6C-D). Next, we examined mature let-7 miRNA expression in CD34⁺ cord blood cells and K562 cells transduced with ADAR1 WT or an ADAR1^{E912A} mutant (ADAR1^{E912A} Mut), which had reduced RNA editing capacity (FIGS. 6E and 13E-I). Notably, we observed a significant reduction in mature let-7 miRNA levels in cord blood CD34⁺ cells following transduction with ADAR1 WT but not with ADAR1E912A (FIGS. 6E). Moreover, only WT ADAR1 significantly increased the self-renewal and survival capacity of normal cord blood CD34+ cells in colony replating and engraftment assays (FIGS. 6F-G and 13J). In RAG2^{-/-}γc^{-/-} mice transplanted with cord blood CD34⁺ cells, we also observed significantly enhanced engraftment as measured by increased CD45⁺ percentage in bone marrow (FIGS. 6G and 13K). These data suggest that ADAR1 regulates let-7 miRNA biogenesis in an adenosine-to-inosine (A-to-I) editing dependent manner, which has a functional impact on progenitor self-renewal and survival.

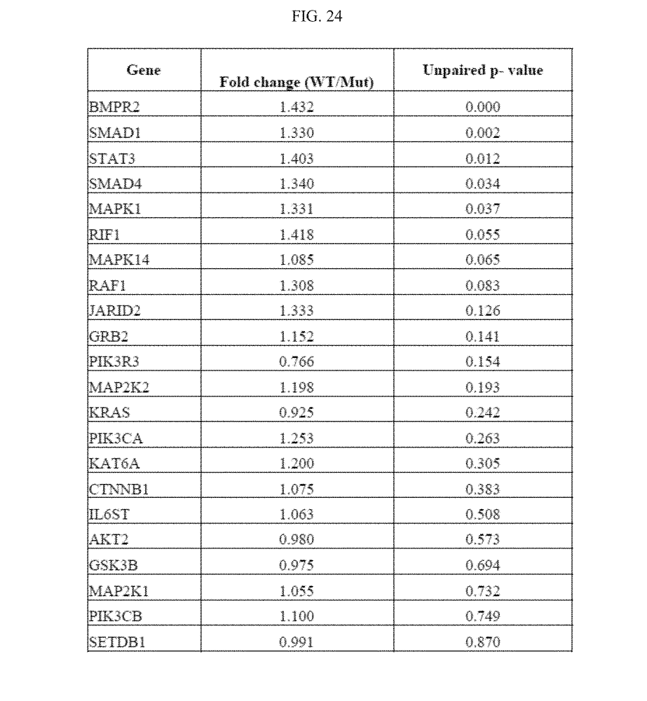

Previous reports indicate that ADAR1 depletion induces stress-related apoptosis during fetal hematopoiesis and loss of mouse HSC multi-lineage repopulating potential (Hartner et al., 2009; Wang et al., 2000; Wang et al., 2004). To investigate the contribution of ADAR1-editing to self-renewal, we performed comparative RNA-seq analysis of ADAR1 WT, ADAR1^{E912A} Mut and backbone transduced human CD34⁺ cells. Volcano plot analysis of RNA-seq data demonstrated distinct differences in expression of known ADAR1 target genes (Roberts et al., 2013) and increased expression of self renewal transcripts following ADAR1 WT compared to ADAR1^{E912A} Mut transduction (FIGS. 6H and FIG. 24, which illustrates Table S5). Table S5 shows data from an RNA-seq analysis of differentially expressed self-renewal genes in ADAR1 WT compared with ADAR1 Mut transduced CD34+ cord blood samples (n=3); p values were calculated using Student-t test.

A comparative analysis of A-to-I editing dependent expression profiles with previously identified differentially expressed transcripts in BC compared with CP (Jiang et al., 2013) identified 38 common transcripts. These included genes involved in self-renewal, such as FBXW7 and MAML2, and miRNA regulation, such as SMAD1 (FIG. 6I). Taken together, these results indicate that ADAR1-mediated A-to-I editing contributes to LSC self-renewal.

### ADAR1 Editase Activity Regulates Let-7 Biogenesis

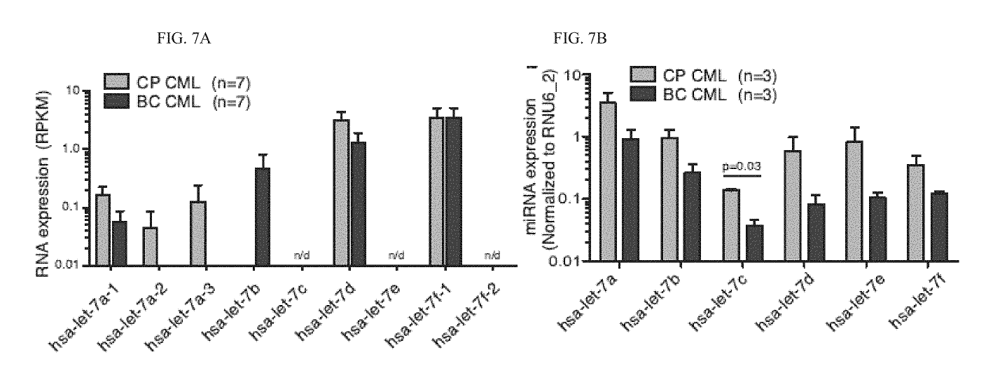

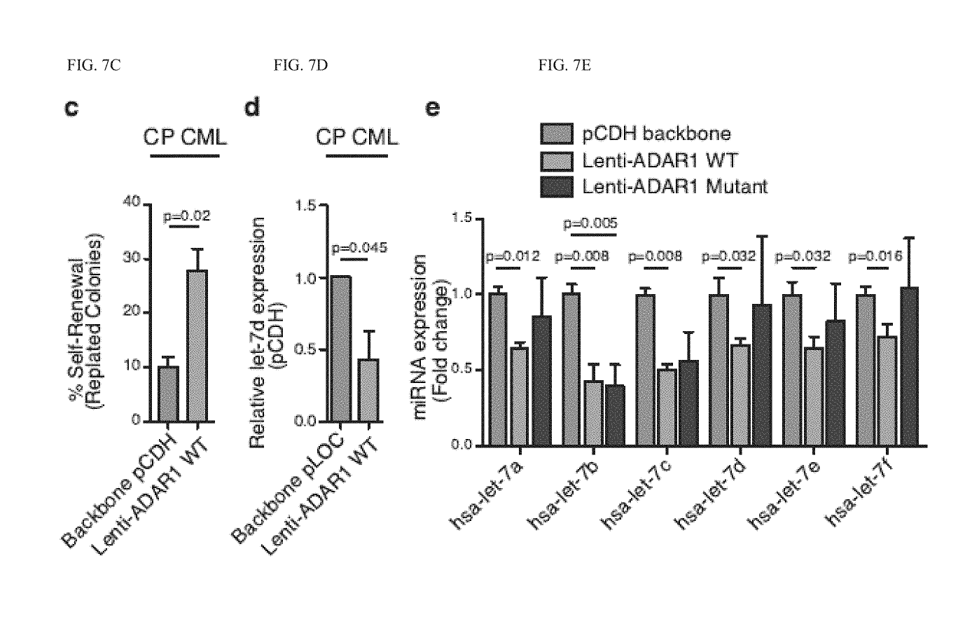

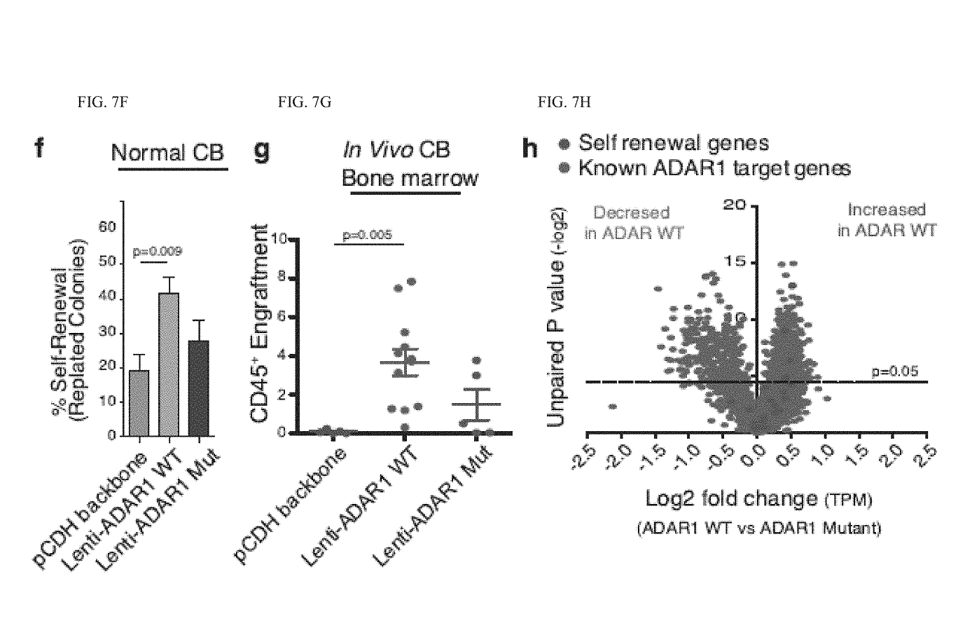

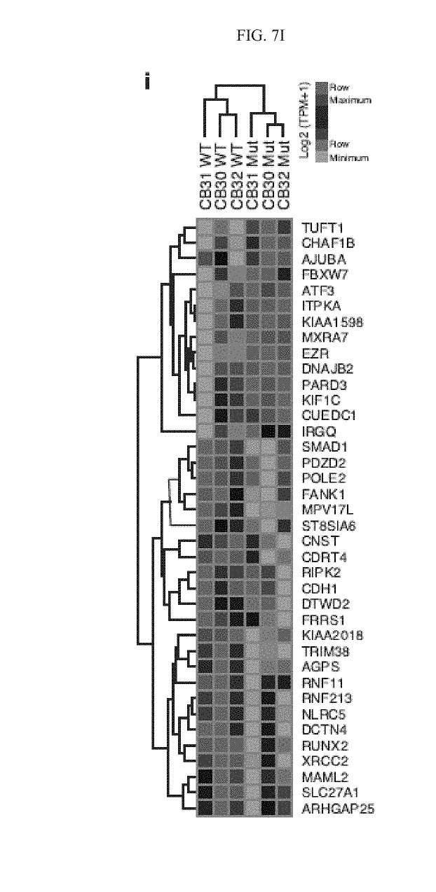

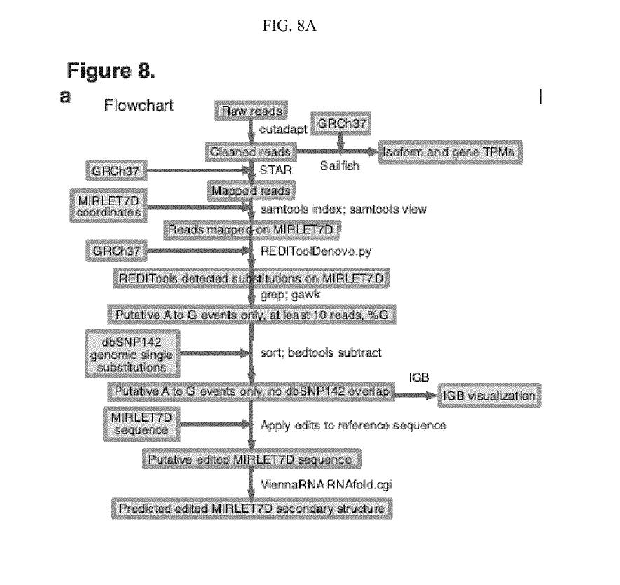

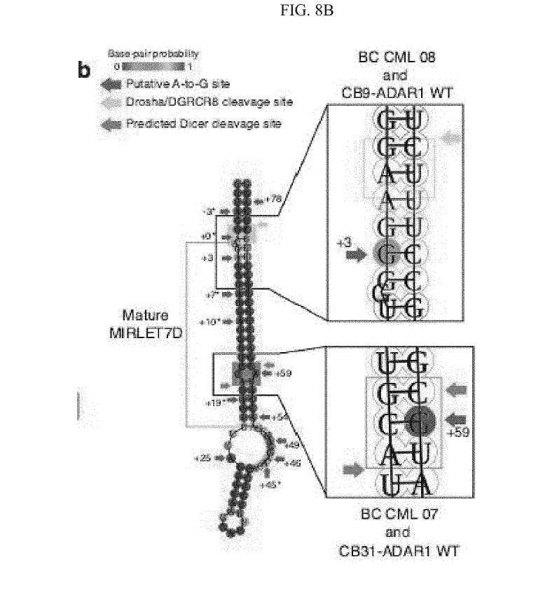

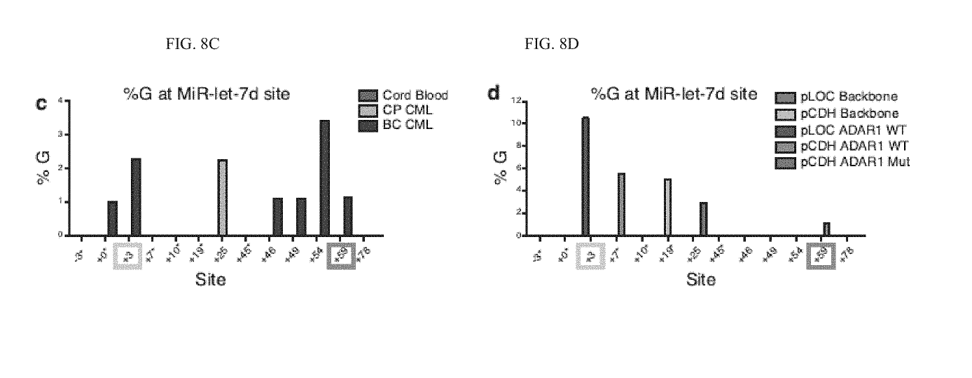

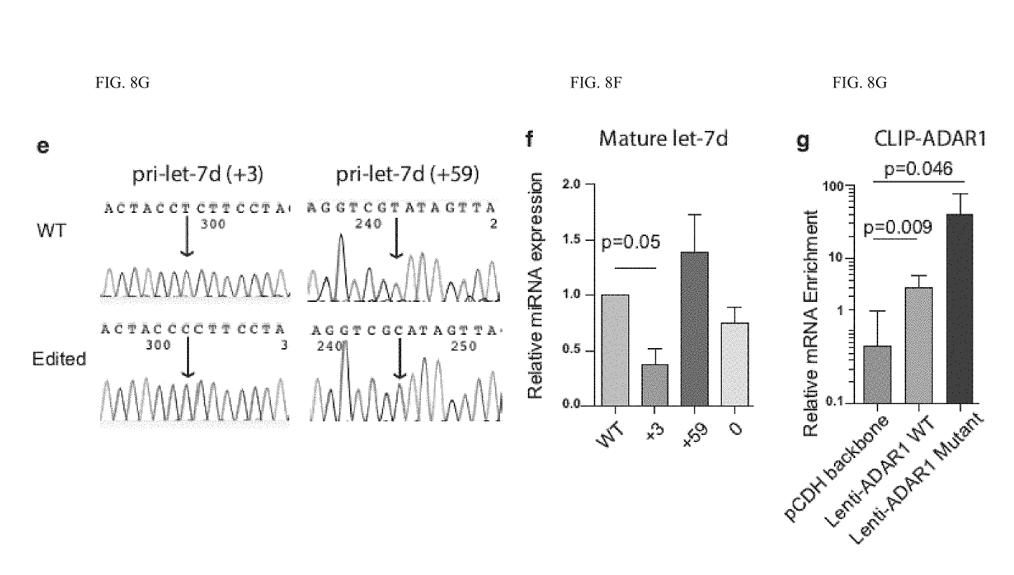

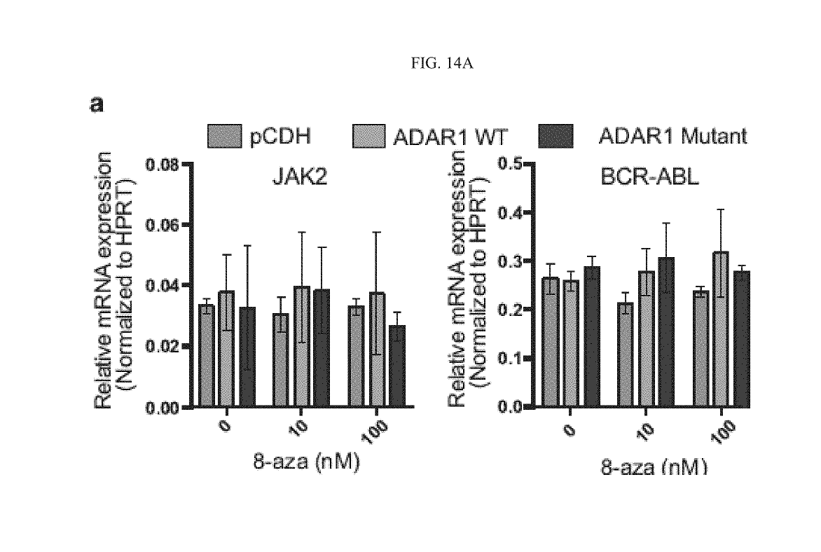

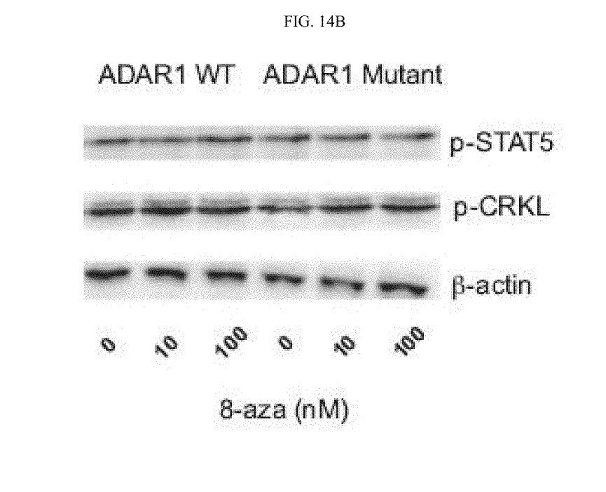

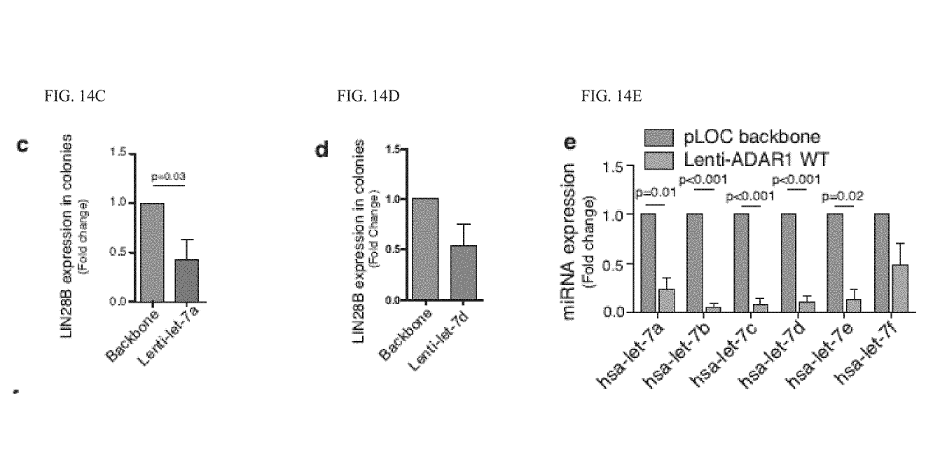

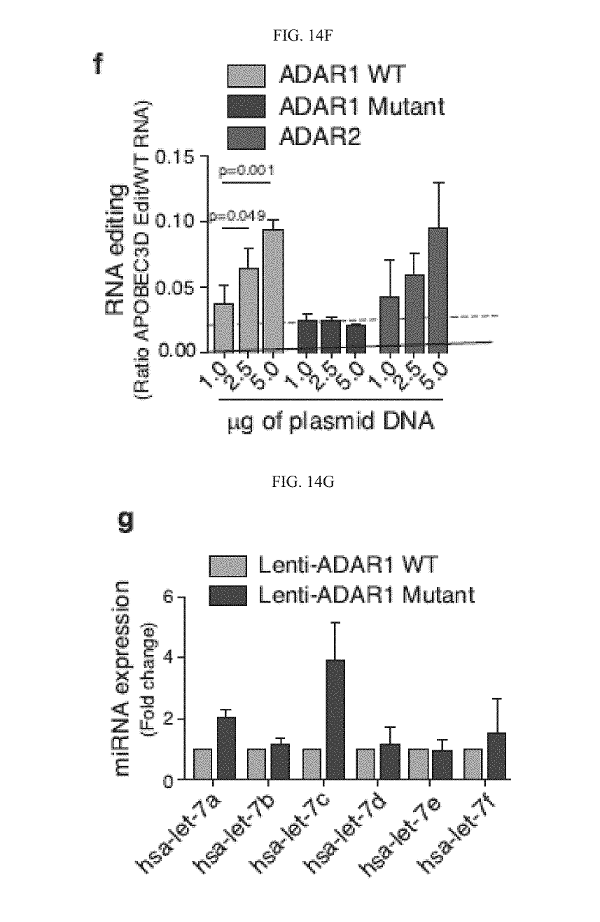

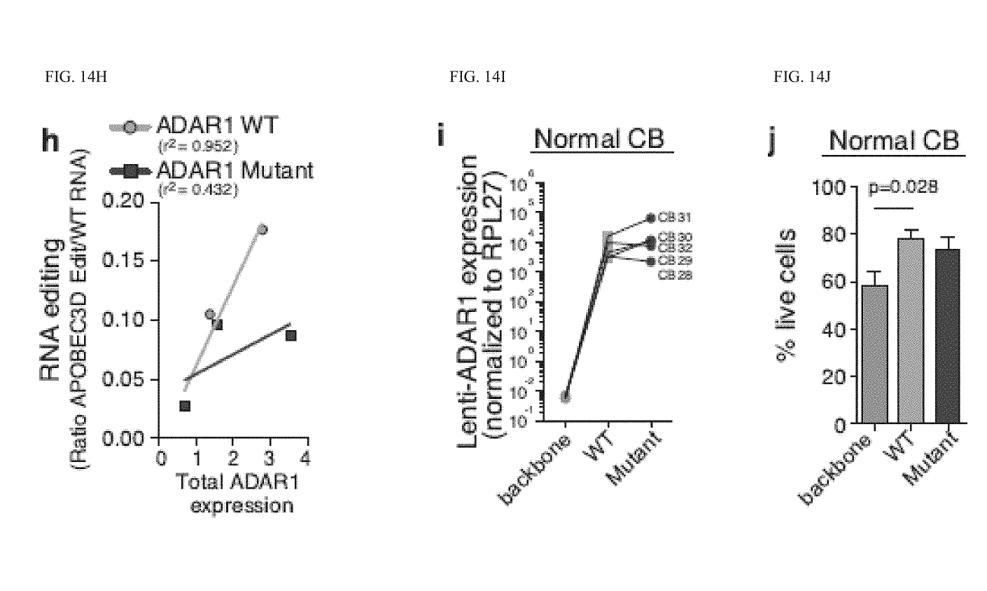

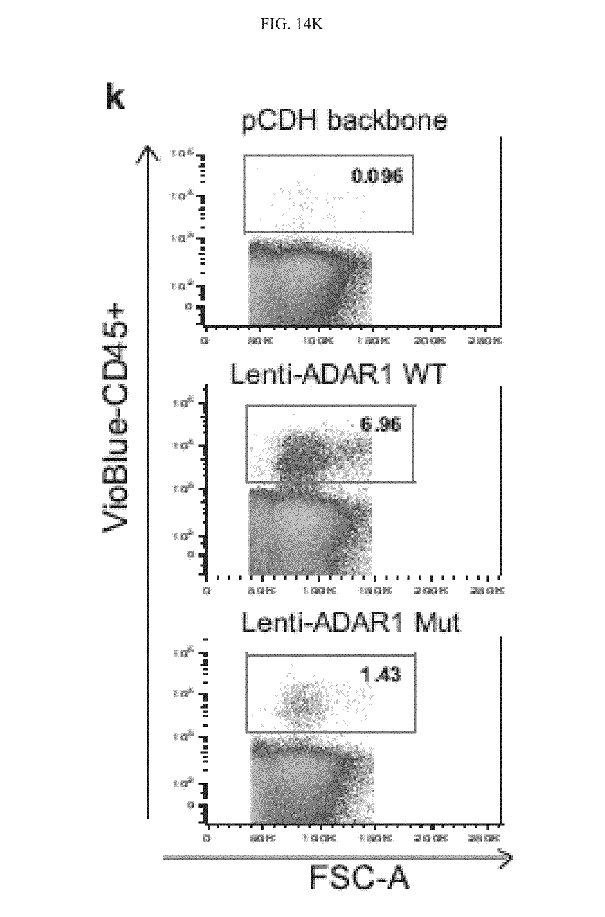

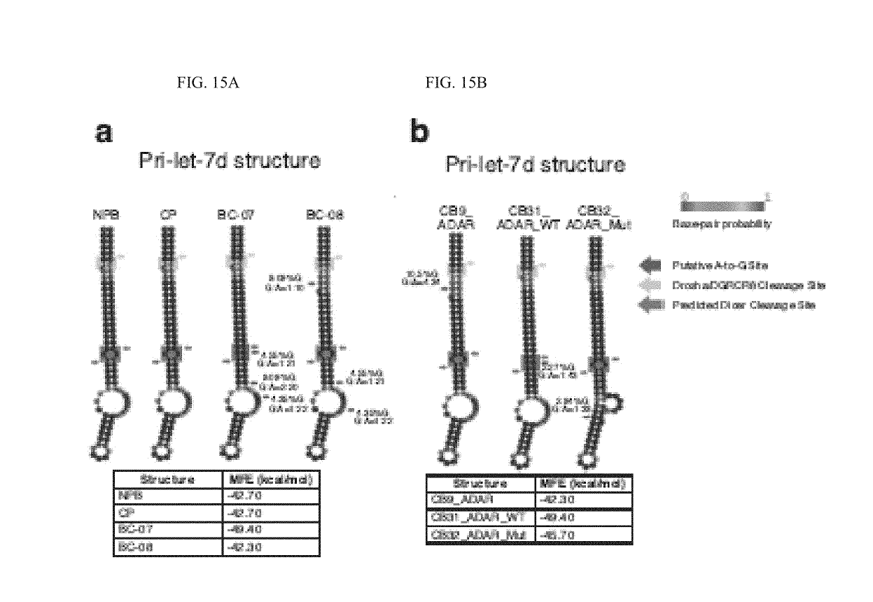

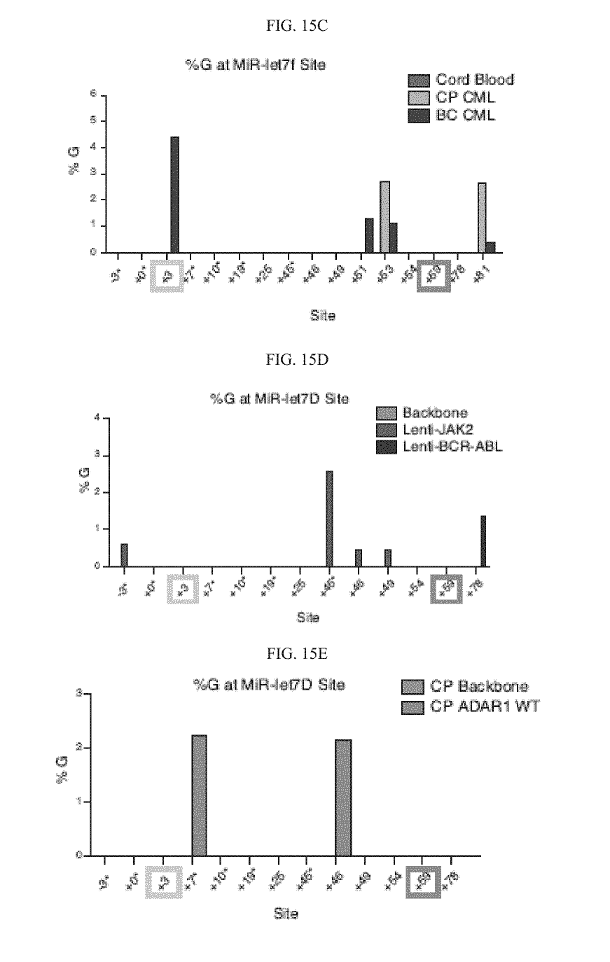

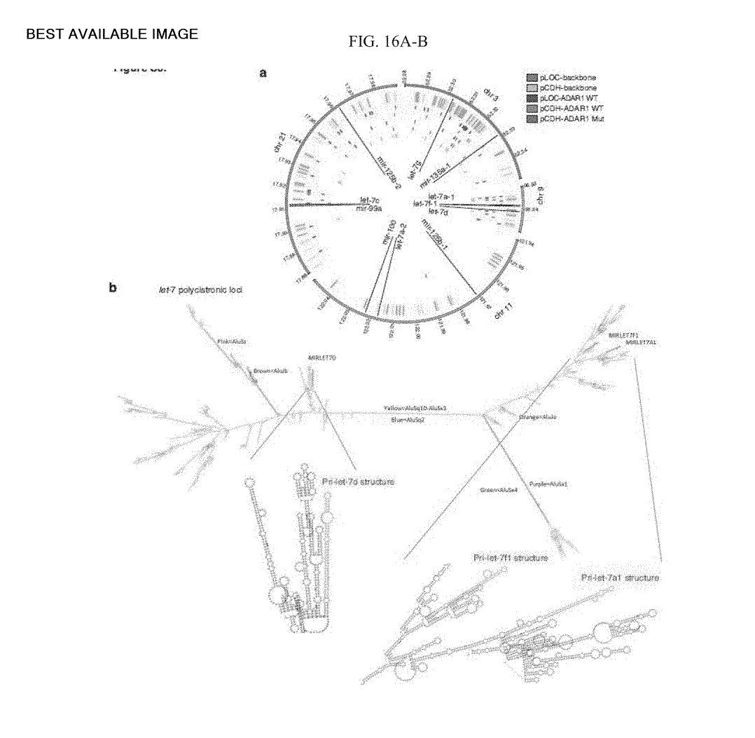

Other studies suggest the ADAR1 mediates miRNA biogenesis by editing polycistronic miRNAs in drosophila (Chawla and Sokol, 2014). Similarly, the primate polycistronic cluster of let-7a, let-7d, and let-7f possesses differential mature miRNA expression potential dependent on ADAR1 RNA editing activity (FIG. 6E). Therefore, the location of adenosine-to-inosine (A-to-I) editing in the let-7 polycistronic cluster was investigated utilizing RNA-seq along with Vienna RNA secondary RNA structure prediction software (Yu et al., 2012) of both primary CML samples and normal progenitors transduced with ADAR1WT, ADAR1^{E912A} Mut, JAK2, or BCR-ABL1 lentiviral vectors (FIGS. 7A-B and 14A-B). While a few editing sites were detected in pri-let-7f in CP and BC progenitors (FIG. 14C), pri-let-7d was found to be edited at multiple locations (FIGS. 7C-D and 14D-E). Notably, A-to-G nucleotide changes at the +3 and +59 editing sites were predicted to alter RNA secondary structures at the DROSHA/DGCR8 and DICER cleavage sites, respectively (FIGS. 7B and 14A-B). Both increased and differential A-to-G editing sites were detected in BC compared with CP and normal progenitors (FIG. 7C). The highest editing frequency was observed in close proximity to the predicted DROSHA/DGCR8 cleavage site (+3) in ADAR1 WT transduced samples (FIG. 7D). Most importantly, two common editing sites adjacent to DROSHA/DGCR8 and DICER cleavage sites (+3, +59) were detected in BC progenitors and ADAR1 WT transduced cord blood cells, indicative of a pivotal role for ADAR1-mediated editing in BC LSC generation (FIGS. 7C-D).

Previous studies revealed that adenosine-to-inosine (A-to-I) editing near the DROSHA or DICER cleavage sites in pri-miRNAs inhibited the cleavage reaction and reduced mature miRNA biogenesis (Nishikura, 2010; Yang et al., 2006). The effect of A-to-I editing on pri-let-7d biogenesis in 293T cells transfected with edited and unedited pri-let-7d expression plasmids was investigated (FIG. 7E-F). The unedited wild-type (WT) pri-let- 7d plasmid, and “pre-edited” pri-let-7d at +3 and +59 as identified in BC CML progenitors, as well as at 0, which is the DROSHA/DGCR8 cleavage site was examined. Compared to the WT pri-let-7d construct, editing at the +3 site induced a significant reduction in mature let-7d miRNA levels (FIG. 7F). Interestingly, the A-to-G changes in +59 or 0 sites did not show any changes in mature miRNA levels, suggesting that the +3 editing site is responsible for the reduced mature let-7d miRNA expression observed in ADAR1-transduced cord blood progenitors. Lastly, cross-linking immunoprecipitation (CLIP) assays were performed in a K562 leukemic cell line stably expressing ADAR1 WT or ADAR1^{E912A} to determine if ADAR1 directly binds to pri-let-7d transcripts (FIG. 7G). Using an ADAR1 antibody, we were able to isolate pri-let-7d miRNAs in both ADAR1 WT and ADAR1^{E912A} transduced cells, thereby confirming the capacity of ADAR1 to bind to pri-let-7d transcripts. These findings reveal a pivotal role for ADAR1 editase activity in let-7d biogenesis and BC LSC self-renewal. Further analysis of let-7 clusters demonstrated increased A-to-G editing events within primate-specific Alu repeat sequences with a proclivity for forming dsRNA loops (FIG. 15A). Single nucleotide resolution RNA sequencing analysis revealed that pri-let-7d is located in close proximity to multiple Alu repetitive sequences, which may enhance the capacity of ADAR1 to bind and edit pri-let-7d (FIG. 15B).

### Discussion

Malignant RNA editing conferred by ADAR1 activation has emerged as a dominant driver of cancer relapse and progression (Jiang et al., 2013; Qi et al., 2014; Qin et al., 2014; Shah et al., 2009). Moreover, a recent report describing a genome wide analysis of 6,236 patient samples, representing 17 tumor types in the Cancer Genome Atlas database, revealed non-synonymous A-to-I editing events that were predicted to promote therapeutic resistance (Han et al., 2015). These discoveries have fueled intensive research into the cell type and context specific mechanisms driving ADAR1 activation and the impact on self-renewing CSC generation in malignancies that have a proclivity for therapeutic resistance and progression.

In particular, the oncogenic drivers of ADAR1 activation, the A-to-I editing targets, and the non-cell autonomous as well as cell autonomous mechanisms that govern CSC self-renewal had not been elucidated. By employing whole-transcriptome sequencing of normal, CP and BC CIVIL patient progenitor samples and human BC CML progenitor serial transplantation mouse models, the disclosure herein provides a novel link between increased sensitivity to JAK2-dependent cytokine signaling and ADAR1 editase mediated generation of self-renewing LSCs. The data herein show that ADAR1 activation in BC LSCs is triggered by increased JAK2-dependent inflammatory signaling and is further amplified by the presence of BC-ABL1. Conversely, pharmacologic inhibition of JAK2 and BCR-ABL1 prevented LSC self-renewal commensurate with reduced BCR-ABL1 and ADAR1 p150 expression in humanized BC LSC mouse model. These data highlight a dual mechanism of malignant RNA editing activation in LSCs.

While genetic ablation of ADAR1 in mice leads to embryonic lethality due to severe defects in erythropoiesis (Wang et al., 2000), conditional deletion in the hematopoietic system impairs long-term hematopoietic stem cell (HSC) maintenance, indicative of key roles for ADAR1 in both cell fate specification and self-renewal (Hartner et al., 2009). This suggests that deregulation of editase activity may play a significant role in a variety of blood disorders that have acquired aberrant stem cell self-renewal characteristics. Indeed, in a humanized mouse model of CML, lentiviral shRNA knockdown of ADAR1 inhibited self-renewal of malignant progenitors that promote blast crisis transformation (Jiang et al., 2013).

Here we advance this further by differential gene expression analysis in order to determine the genes involved in HSC self-renewal. Notably, lentiviral ADAR1 overexpression significantly affected genes involved in the regulation of stem cell pluripotency (FIG. 6). Among the upregulated genes in ADAR1 WT compared to backbone transduced normal CD34⁺ cells (FIG. 6C), WNT4 and WNT9a are signaling proteins. The WNT pathway is critical for normal HSC homeostasis and self-renewal (Louis et al., 2008; Reya et al., 2003). In addition, we observed upregulation of genes that regulate the TGF-β pathway, including SMAD1 and SMAD2, which transduce extracellular signals to activate transcription of genes that regulate cellular growth, differentiation, apoptosis (Heldin et al., 1997; Nakao et al., 1997) as well as miRNA expression and maturation (Blahna and Hata, 2012; Davis et al., 2008).

Previously, ADAR1-mediated differential expression of m iRNAs was shown to control gene expression through several mechanisms including direct protein binding with DROSHA and DGCR8, regulation of DICER mRNA expression, and regulation of miRNA biogenesis (Bahn et al., 2015; Nemlich et al., 2013). The LIN28B/Let-7 stem cell regulatory axis plays a critical role in stem cell maintenance (Copley et al., 2013; Wang et al., 2015), and appears to be deregulated in tumorigenesis (Melton et al., 2010; Piskounova et al., 2011; Viswanathan et al., 2008). Here, we show that by impairing let-7 biogenesis ADAR1 enhances LSC self-renewal. Combined inhibition of ADAR1 and JAK2 restores let-7 expression and inhibits LSC self-renewal. Since ADAR1 mediates differential expression of polycistronic miRNAs transcribed from the lethal-7-Complex (let-7-C) locus by altering DROSHA processing (Chawla and Sokol, 2014), we performed single nucleotide resolution RNA-seq combined with secondary RNA structure prediction (ViennaRNA software) (Yu et al., 2012) analyses and miRNA qRT-PCR. These analyses demonstrated that BC CML and ADAR1 transduced progenitors harbored enhanced editing at the polycistronic let-7 loci and reduced mature let-7 microRNA levels (Melton et al., 2010; Yu et al., 2007), Let-7 loci outside the polycistronic cluster displayed no editing signatures in ADAR1 transduced progenitors. Moreover, RNA editing adjacent to the +3 DROSHA/DGCR8 cleavage site was associated with reduced let-7d biogenesis in BC LSCs and CD34⁺ progenitors transduced with ADARI but not with ADAR1^{E912A} and empty vectors. Finally, CLIP-ADARI assays combined with site directed mutagenesis mediated introduction of let-7d edits confirmed that ADAR1 directly binds and edits pri-let-7d transcripts thereby reducing the expression of mature let-7d miRNA as measured by qRT-PCR.

The disclosed herein show that ADAR1 editase activity impairs let-7 family miRNA biogenesis and increases progenitor self-renewal capacity resulting in malignant reprogramming of progenitors into BC LSCs. In addition, it is shown that enhanced sensitivity to cytokine signaling as a consequence of JAK2-responsive cytokine receptor upregulation and BCR-ABL1 oncogene amplification results in ADAR1 activation. While previous studies have shown that JAK2 signaling is important in the induction of numerous transcriptional mediators, the discovery disclosed of a pivotal JAK2-ADAR1-let-7 self renewal axis provides the first mechanistic link between inflammatory cytokine-driven oncogenic signaling pathways and RNA editing-driven malignant reprogramming of progenitors into LSCs. Perhaps most importantly, targeted reversal of ADAR1 activity may impede the generation of cancer stem cells in a broad array of therapeutically recalcitrant malignancies that evolve in inflammatory microenvironments.

ADAR1 RNA editase enhances oncogenic transformation and it is able to directly edit APOBEC3 (apolipoprotein B mRNA editing enzyme catalytic polypeptide-like) DNA deaminases. Recent studies have shown that ADAR1 RNA editase enhances oncogenic transformation and it is able to directly edit APOBEC3 (apolipoprotein B mRNA editing enzyme catalytic polypeptide-like) DNA deaminases, which induce DNA mutagenesis and therapeutic resistance in many human malignancies. Applying the disclosure herein RNA and DNA editing signatures, induced by ADAR1 and APOBEC deaminases, can be early predictive biomarkers of pre-leukemic MPN progenitor transformation into self-renewing, therapy resistant LSCs.

After RNA-sequencing data of normal progenitors (cord blood CD34⁺) transduced with ADAR1, we noticed two hotspots of ADAR1 activity with clusters of A-to-I editing sites in close proximity to Alu elements in APOBEC3F and APOBEC3G transcripts. The average of editing efficiency (% G) is approximately 15-20%. The ADAR1 E/A deamination inactivated mutant also exhibits some A-to-I editing activity, though much less frequent than the ADAR1 WT transduced progenitors. In contrast, other APOBEC3 family members, including APOBEC3A, APOBEC3C, and APOBEC3D showed limited to no A-to-I editing sites. This provides the first evidence that an RNA editor, ADAR1, may edit a DNA editor, APOBEC3. Since activation of ADAR1 RNA editing is required for leukemia stem cell (LSC) self-renewal and CIVIL progression (Jiang, et al, 2013, PNAS), the disclosure herein indicates that ADAR1's RNA editing signatures in APOBEC3 transcripts is useful as predictive biomarkers of LSC generation and MPN disease progression, drug screening for treatment of LSC associated diseases, and as a marker for initiating treatment and monitoring treatment success.

RNA-sequencing data revealed that the expression of APOBEC3F and APOBEC3G (A3F, A3G) is upregulated during progenitor transformation from CP to BC CML LSC. Next, we examined the A-to-I editing signature of A3F and A3G in progenitors of NPB controls, CP CML, and BC CML to assess if the editome of A3F and A3G are potential biomarkers of LSC generation and disease progression. Although the same clusters of hyper-editing was observed in all progenitor populations, BC progenitors clearly possess distinct editome signatures where the enhanced editing level, some at 100% G, and increased editing sites. The data suggests the A-to-I editomes of A3F and A3G are biomarkers raised during disease progression due to LSC generation. The A-to-I editing sites in A3F and A3G are presented in intronic, exonic and protein-coding regions, suggesting ADAR1 might regulate both expression and protein function. Indeed, several A-to-I editing sites unique to BC CML progenitor editing signature are located with protein coding region of A3F and A3G. Without wishing to be bound to any particular theory, the data may indicate that the A3F and A3G mutants identified in BC CML LSC and sAML LSC, have differential function in encouraging the self-renewal capacity of chronic phase Ph⁺ and Ph⁻ myeloproliferative neoplasms (MPN) progenitors. Thus, the APOBEC3-mediated DNA deamination signatures can be used as predictive biomarkers of LSC generation and MPN disease progression.

### Experimental Procedures

Primary normal and CIVIL samples were obtained and RNA-Seq analysis as well as qRTPCR validation were performed according to published methods (Abrahamsson et al., 2009; Goff et al., 2013; Jiang et al., 2013). MiRNAs were extracted using a RNeasyMicro Kit and qRT-PCR was performed using miRNA human-specific primers normalized to RNU6_2 and SNORD44. Lentiviral human wild-type JAK2, BCR-ABL1, wild-type and mutant ADAR1E912A (overexpression vectors were produced in the pCDH-EF1-T2A-GFPor pLOC lentiviral vector systems). Progenitor transduction, SL/M2 co-culture and colony assays were performed as previously described (Abrahamsson et al., 2009; Goff et al., 2013; Jiang et al., 2013). Immunocompromised RAG2^{-/-}γc^{-/-} mice engrafted with human BC CML progenitors were treated for two weeks with SAR302503, Dasatinib or the combination followed by FACS analysis of human progenitor engraftment in hematopoietic tissues and serial transplantations. The RNA-sequence data accession numbers are PRJNA319866 and PRJNA214016.

### RNA and MicroRNA Extraction and Quantitative RT-PCR

Total RNA was isolated from 20,000 to 50,000 FACS-sorted or CD34⁺ selected (MACS) progenitors cells from normal cord blood, CP CML, BC CML or from xenografted mice and complementary DNA was synthesized according to published methods (Abrahamsson et al., 2009; Goff et al., 2013; Jiang et al., 2013). Then qRT-PCR was performed in duplicate on an iCycler with the use of SYBR GreenER qPCR SuperMix™ (Invitrogen), 5ng of template mRNA and 0.2 μM of each forward and reverse primer, as illustrated in FIG. 25.

Human specific RPL27 primers were used as housekeeping control. MicroRNA extraction was performed using the RNeasy Micro Kit (Qiagen) according to the manufacturer's instructions. Then 30 ng of cDNA was prepared in a reverse transcription reaction using miScript RTII™ kit (Qiagen) and served as a template for the quantification of the expression of mature miRNA of interest. Also, qRT-PCR was performed using miRNA human-specific primers and SYBR Green Kit (Qiagen). MiScript™ primers, RNU6_2 (Qiagen), were used as housekeeping control. The expression of primary and precursor miRNA transcripts were measured using previous published primers (Patterson et al., 2014).

### Cross-Linking and Immunoprecipitation (CLIP)

CLIP was performed using a previously published protocol with modification (Bahn et al., 2015). K562 cells (10⁷) were harvested and washed with ice-cold PBS twice. Crosslinking was performed with paraformaldehyde at a final concentration of 0.3% for 5 minutes at room temperature (RT), and the reaction was quenched by glycine. Cell were lysed in 1XPBS, 0.256M Sucrose, 8 mM Tris-HCL (pH7.5), 4 mM MgCl2, and 1% Triton X-100. After 15 minute lysis on ice, cells were sonicated at 10 s three times with 1-minute intervals and centrifuged at 13,000 g, 4° C. for 10 minutes. Supernatant was treated with100 U RNases-free DNase (Roche) at 37° C. for 30 minutes and centrifuged at 13,000g, 4° C., for 10 minutes. ADAR1 antibody (ab168809, Abcam) was added to a final concentration of 20 μg/mL and incubated overnight at 4° C. Dynabeads™ Anti-Rabbit IgG (50-100 μL) was added and incubated with samples for 4 hours at 4° C. on the rotating rocker. Samples were washed twice with CLIP buffer (150 mM KCl, 25 mM Tris-HCl pH7.4, 5 mM EDTA, 0.5 mM DTT, 0.5% NP40, 100 U/mL RNases inhibitor, and protease inhibitor). Samples were treated with Proteinase K (Roche) before being harvested with RLT buffer for mRNA and miRNA extraction as described.

### Human Progenitor Xenotransplantation and Treatment

Immunocompromised RAG2^{-/-}γc^{-/-} mice were bred and maintained in the Moores Cancer Center vivarium according to IACUC approved protocols. Neonatal mice were transplanted intrahepatically with 20,000-100,000 BC CML or human cord blood CD34⁺ cells according to published methods (Abrahamsson et al., 2009; Goff et al., 2013; Jiang et al., 2013). Transplanted mice were FACS screened for human engraftment in peripheral blood at 6-10 weeks. Engrafted (>1% human CD45⁺ cells) mice were treated by oral gavage with SAR302503 (Sanofi-Aventis) twice daily with 60 mg/kg (0.5% methylcellulose, 20% tween 80 and H2O), 50 mg/kg dasatinib daily (50% propylene glycol, 50% PBS), combination (SAR302503 plus dasatinib), or drug vehicles for two weeks. Following treatment, mice were euthanized and single cell suspensions of hematopoietic tissues were analyzed by FACS for human engraftment and 20,000-100,000 human CD34+ cells were serially transplanted into neonatal RAG2^{-/-}γc^{-/-} mice. A subgroup of mice was treated for 2 days, and progenitor cells in the bone marrow were analyzed by qRT-PCR.

### A-to-G SNV Coordinates Analysis

Variants were called from RNA seq data using the GATK pipeline for calling variants in RNA-seq (https ://www.broadinstitute. org/gatk/guide/article?id=3891). Two-pass alignment was performed on paired-end reads using STAR (Qian et al., 2010), against the GRCh37/hg19 reference genome, with the GRCh37.75 annotation input as the initial splice junction database. The resulting reads were sorted and marked for duplicates using Picard™ (http://picard.sourceforge.net). The GATK tool SplitNCigarReads™ was used to reduce false positives due to inaccurate read splicing. GATK was also used to realign reads locally around Indels and to recalibrate base qualities (Li et al., 2014). The GATK Unified Genotyper and Haplotype callers were used to call variants in VCF format, which were then annotated using SNPEff for predicted gene effects (Rampal et al., 2014). The called variants were filtered using SnpSift to only include A to G variants not included as single genomic events in dbSNP138. The resulting coordinates were visualized as tracks in a Circos plot, focusing on coordinates for let-7 clusters from mirBase (GRCh38 coordinates mapped back to GRCH37/hg19) (Tallawi et al., 2014).

### Predicted Cleavage Sites

Using PHDCleav (Tahira et al., 2011) with the GRCh37 MIRLET7D reference sequence as input, candidate DICER cleavage sites were found 24-25 nucleotides from the pre-let-7d 5′ end. The secondary structure at those coordinates matched ViennaRNA RNAFold structure predictions, and a structure given by Heo et al., 2012 (Hu et al., 2011). Heo et al., 2012 also indicates DROSHA/DGCR8 cleavage sites for MIRLET7D. Both cleavage sites were visually annotated on the predicted edited MIRLETD secondary structures.

### Predicted Polycistronic Transcript Secondary Structure

For cord blood samples, each with pCDH backbone, pCDH-ADAR1 WT, and pCDHADAR1E912A mutant, reads mapped to the GRCh37 reference were indexed using samtools, then samtools view (Han et al., 2014) was used to extract all reads from chromosome 9. Cufflinks™ was used with the cufflinks-compatible Igenomes Ensemble GRCh37™ GTF file to perform reference-guided transcript assembly and cuffmerge was used to merge the assembled transcripts (Danielson et al., 2015). The transcript overlapping the let-7 cluster interval was identified, and its sequence was extracted using GFFRead™. ViennaRNA RNAfold was used to predict the secondary structure of the merged polycistronic transcript. The secondary structure was then annotated with the human chromosome 9 let-7 cluster members, and with Alu regions taken from the “alu.bed” annotation used by Conti et al., 2015 (Raval et al., 2007).

### Patient Sample Preparation and FACS Sorting

Normal cord blood and adult peripheral blood samples were purchased from AllCells (Emeryville, Calif.) and Lonza (Allendale, N.J.). Primary CML samples were obtained from consenting patients at the University of California San Diego, Stanford University, the University of Toronto Health Network and the University of Bologna according to Institutional Review Board approved protocols. CD34⁺ cells were enriched from mononuclear fractions by immunomagnetic bead separation (MACS; Miltenyi, Bergisch Gladbach, Germany) followed by FACS purification of hematopoietic stem and progenitor cells. As previously described (Abrahamsson et al., 2009; Goff et al., 2013; Jiang et al., 2013), CD34-selected cells were stained with a mixture of lineage antibodies (fluorescent conjugated CD2, 3, 4, 8, 14, 19, 20, 56) to identify the lineage-negative (Lin⁻) fraction. Subsequently, cells were washed and stained with a mixture of antibodies specific for myeloid progenitors including human CD45-V450, CD34-APC, CD38-PECy7, CD123-PE and CD45RA-FITC (all from BD Biosciences, Franklin Lakes, N.J.). All antibodies were diluted at 1:50 in 2% FBS/HBSS (staining media). After staining, cells were washed and resuspended in PI (1:1000 in staining media). For RNA-Seq analyses, FACS-purified progenitor cells from normal cord blood, CP CML, or BC CML samples were sorted directly into RLT buffer (Qiagen, Valencia, Calif.) as described previously (Abrahamsson et al., 2009; Goff et al., 2013; Jiang et al., 2013). Lysates were processed for RNA and miRNA extraction using RNeasy Micro™ kits (Qiagen).

### Nanofluidic Phospho-Proteomic Immunoassay

Nanoproteomics experiments were performed with the Nanopro 1000™ instrument (Cell Biosciences) and samples were run in triplicate. Briefly, for each capillary analysis, 4 nl of 10 mg/ml lysate was diluted to 0.2 mg/ml in 200 nl HNG (20 mM HEPES pH 7.5, 25 μM NaCl, 10% glycerol, Sigma Phosphatase Inhibitor Cocktail 1 diluted 1:100 and Calbiochem Protease Inhibitor diluted 1:100). Then 200 nl sample mix containing internal pI standards was added. The Firefly™ system first performed a charge-based separation (isoelectric focusing) in a 5-cm-long, 100-micron-inner-diameter capillary. Predicted pIs were calculated optimize the resolution of different peak patterns. The peaks represent antibody signals detected using pJAK2, JAK2, pSTAT5a and B2-microglobulin (B2M) specific antibodies, after separation and photoactivated in-capillary immobilization. Peaks were also quantified by manually selecting the start and end of each peak and a flat baseline and calculating the area under the curve (AUC).

### RNA Editing Site-Specific qRT-PCR (RESSq-PCR) Assay

As previously described (Yildirim et al., 2013), for each RNA editing site, two sets of primers were used: one pair detecting the WT transcript (an “A” base), and one pair detecting the edited transcript containing a “G” base representing inosine substitution. The forward (FW) outer and reverse (REV) outer primers flank the editing site and can be used for Sanger sequencing validation of each editing site, and also as a qRTPCR positive control to ensure that the editing region is detectable in cDNA. The 3′ ends of the FW inner and REV inner primers match either the WT A or edited G nucleotide, and an additional mismatch was incorporated two nucleotides upstream of the 3′ primer end to enhance allelic discrimination, as previously described for quantitative detection of transcripts harboring single nucleotide genomic mutations. RESSqPCR was performed as recently described (Yildirim et al., 2013) using highly validated RNA editing site specific primers for APOBEC3D. Samples were analyzed in duplicate using cDNA (1 μL reverse transcription product per reaction), prepared from DNase-digested RNA extracts, on an iCycler™ (Bio-Rad) using SYBR GreenER Super Mix™ (Life Technologies) in 254, volume reactions containing 0.2 μM of each forward and reverse primer. Relative RNA editing rates (Relative edit/WT RNA) were calculated using the following calculation: 2\hat{ }-(Ct Edit-Ct WT).

### Generation of ADAR1 and ADAR2 Constructs

Full-length human adenosine deaminase, RNA-specific (ADAR1), transcript variant 1, was subcloned into Gateway® entry vector pDONRT™221 by two rounds of PCR using attB modified custom primers from pReceiver-M02-ADAR1 (cat# EX-00744-M02, GeneCopoeia, Rockville, Md.).

The first round of PCR primers used was forward primer: B1-TEV-ADAR1 (5′-TAT TTT CAG GGC ATG AAT CCG CGGCAG-3′) (SEQ ID NO:1) and reverse primer: B2-ADAR1FLAG (5′-GTC GTC CTT GTA GTC TAC TGG GCA GAG-3′) (SEQ ID NO:2).

The second round of PCR primers used was forward primer: attB1-TEV: (5′-GGGG ACA AGT TTGTAC AAA AAA GCA GGC TCC GAG AAT CTT TAT TTT CAG GGC-3′) (SEQ ID NO:3) and reverse primer: attB2-FLAG (5′-GGGG ACC ACT TTG TAC AAG AAA GCT GGG TA TTA CTT GTC ATC GTC GTC CTTGTA GTC-3′) (SEQ ID NO:4).

pDONR™221-ADAR1FLAG was recombined with pDEST™26 mammalian expression vector to generate pDEST26-ADAR1-FLAG. Full-length human adenosine deaminase, RNA-specific, B1 (ADAR2) was subcloned into Gateway® entry vector pDONR™221 by two rounds of PCR using attB modified custom primers from pCMV-SPORT-ADAR2 (cat.# MHS6278-202759234,GE Dharmacon, Lafayette, Colo.).

The first round of PCR primers used was forward primer: B1-TEV-ADAR2 (5′- TAT TTTCAG GGC ATG GAT ATA GAA -3′) (SEQ ID NO:5) and reverse primer: B2-ADAR2FLAG (5′- GTC GTC CTT GTAGTC GGG CGT GAG TGA -3′) (SEQ ID NO:6).

The second round of PCR primers used was forward primer: attB1-TEVand reverse primer attB2-FLAG as above. pDONR™221-ADAR2FLAG was recombined withpDEST™26 mammalian expression vector to generate pDEST26-ADAR2-Flag. Both pDEST26-ADAR1-FLAG and pDEST26-ADAR2-FLAG constructs were verified by DNA sequencing. Oligonucleotide primers were synthesized using ValueGene (San Diego, Calif.). Generation of ADAR1 mutant and pri-let-7d mutant constructs Production of the catalytically inactive ADAR1 E912A was performed as previously described (Crews et al., JTM, 2015). For production of the edited primary let-7d transcripts, site directed mutagenesis was performed using the QuikChange II Site-Directed Mutagenesis Kit™ (Agilent) according to manufacturer's instructions. Mutagenic primers were designed to introduce an A-to-G substitution at either the -3, 0, +3 or+59. Primers contained the desired mutation and annealed to the same sequence on opposite strands of the plasmid (FW is forward primer and REV is reverse primer):

let-7d -3 FW
(SEQ ID NO: 7)
GCA AGA AAA AAA AAA TGG GTT CCT GGG AAG AGG
TAG TAG GTTGC,
let-7d -3 REV
(SEQ ID NO: 8)
GCA ACC TAC TAC CTC TTC CCA GGA ACC CAT TTT
TTT TTT CTT GC,
let-7d0 FW
(SEQ ID NO: 9)
AAA AAA AAT GGG TTC CTA GGG AGA GGT AGT AGG
TTG CAT AG,
let-7d 0 REV
(SEQ ID NO: 10)
CTATGC AAC CTA CTA CCT CTC CCT AGG AAC CCA
TTT TTT TT,
let-7d FW
(SEQ ID NO: 11)
AAT GGG TTC CTAGGA AGG GGT AGT AGG TTG CAT
AG,
let-7d REV
(SEQ ID NO: 12)
CTA TGC AAC CTA CTA CCC CTT CCTAGG AAC CCA
TT,
let-7d +59 FW
(SEQ ID NO: 13)
TGC CCA CAA GGA GGT AAC TAT GCG ACC TGC TGC,
let-7d +59 REV
(SEQ ID NO: 14)
GCA GCA GGT CGC ATA GTT ACC TCC TTG TGG GCA.

XLI super competent cells were transformed with amplification products, after digestion with DpnI. Colonies were screened to identify mutated clones by DNA sequencing (Sanger sequencing, Eton Bioscience).

### Dual Luciferase Reporter Assay and Detection of ADAR-Specific RNA Editing Activity

The human BC CML cell line, K562 was stably transduced with a lentiviral vector expressing humanADAR1 p150 to establish a BCR-ABL⁺ cell line harboring high levels of ADAR1 p150 expression. These K562 cells were then transfected with a dual editing activity, or a constitutively-edited positive control reporter (both provided by Dr. Stefan Maas, Lehigh University), using Amaxa nucelofection technology according to the manufacturer's instruction (Lonza). After transfection with reporter constructs, cells were transduced with lentiviral vectors driving human JAK2 over-expression or the vector control (pCDH-GFP backbone). Forty-eight hours after transduction, 100,000 cells were plated in 12-well plates and treated with SAR302503 at concentrations of 100 nM or 300 nM for 1 or 3 hours. Cells were washed and lysed with cell culture lysis buffer (Promega) and luciferase activity was measured using a dual luciferase assay kit (Promega) by luminescence using a96-well plate reader (PerkinElmer Envision Plate Reader). Both Firefly and Renilla luciferase activities were determined, and all values were normalized for transfection efficiency by dividing the Renilla values with the Firefly luciferase values (relative luciferase activity) as previously described (Gommans et al., 2010). For luciferase reporter assay using increasing amounts of either pDEST26-ADAR1 or pDEST26-ADAR2constructs, leukemic K562 cells were transfected using the Cell Line Nucleofector® Kit V, Program T-016™ (Lonza, Cologne, GER) according to the manufacturer's instructions. After 36 hours of transfection, cellular extracts were collected for renilla and luciferase assays. Renilla assays for MK-Reporter were performed in duplicate and results were normalized to co-expressed luciferase. Notably, A-to-I editing of the reporter promoter drives expression of the renilla reporter gene. Renilla and luciferase activity was measured with a GloMax®-96™ Microplate luminometer, kindly utilized from Dr. LaSpada (Promega, Madison, Wis.), using Dual-Luciferase® Reporter Assay System (Promega, Madison, Wis.).

### Transcript and Gene TPM Quantifications

Starting with raw paired-end fastq files, overrepresented non-genomic sequences (e.g. adapters) were identified with FastQC, then removed using cutadapt (Zhou et al., 2014a). A reference transcriptome fasta was assembled by passing the GRCh37.75/hg19 fasta and Ensembl (Flicek et al., 2010) Gene Transfer Format (GTF) file to gff read, then this transcriptome was indexed by Sailfish™ (Ho et al., 2014). Sailfish™ was then run on the cleaned reads to yield per-transcript quantifications, including Transcripts Per Million (TPM), which were then summed by gene to yield per-gene quantifications. The LogTPM transformation of Log2(TPM+1) (Karlic et al., 2014) was applied prior to Gene Set Enrichment Analysis (Patil et al., 2014) on KEGG pathways (Claus et al., 2012; Wu et al., 2011) and student t-tests. To select only values corresponding to let-7-targets, the file hsa_MTI.xlsx was downloaded from miRTarBase™ (Mudunuri et al., 2009), then converted to CSV. This converted file was searched using grep to find lines referencing genes regulated by has-let-7 microRNAs, and from each extracted line the column corresponding to the Entrez™ gene ID was extracted. The Entrez™ IDs were sorted then pruned with uniq to remove duplicate entries, followed by conversion of Entrez™ gene IDs to Ensembl™ IDs using bioDBnet'sdb2db™ utility. These Ensembl™ IDs of let-7 target genes were used to reference and extract entries from tables containing values for all Ensembl™ listed genes.

### Predicted Edited MIRLET7D Secondary Structure

STAR (Qian et al., 2010) was used to create a reference index from the GRCh37 fasta and GTF. STAR was used for two-pass alignment of the cutadapt-cleaned reads to the GRCh37 reference index with output in coordinate sorted bam format, and also output coverage bedgraph files, and unmapped reads. Using samtools index and samtools view (Han et al., 2014), the coordinate-sorted bam file was filtered for reads that mapped to the interval of MIRLET7D as determined by GRCh37. These reads for the region of interest were passed to REDITools™ (Eades et al., 2014; Li et al., 2014a) (REDIToolDenovo.py -c2 -m10 -q10), along with the reference GRCh37 fasta. This yielded a table of all substitutions at all MIRLET7Dcoordinates, and per-coordinate base counts. Using grep and gawk, only putative A-to-G substitutions with more than 10 reads counted A or G were selected and converted to a bed format (which includes per coordinate base counts and % G base counts). This bed file was sorted and coordinates from dbSNP142 “genomic single” events (downloaded from the UCSC Table Browser, selected using grep, and converted to bed with gawk) were removed using bedtools subtract (Zhou et al., 2014b). The resulting putative A-to-G substitutions were visualized using Integrated Genome Browser (Cheng et al., 2015). These substitutions were applied to the reference GRCh37 MIRLET7D sequence per sample, then the resulting edited sequences were sent to ViennaRNA RNAfold13 web interface for secondary structure prediction and minimum free energy of the secondary structure.

### SL/M2 Co-Culture

The mouse bone marrow stromal cell lines SL and M2 were maintained according to previously published methods (Goff et al., 2013). One day prior to co-culture, the cell lines were treated with mitomycin-C (1 mg/mL for 3 hours) and plated in a 1:1 mixture at a total concentration of 100,000/ml. Normal or CMLCD34⁺ cells were selected and plated on top of the adherent SL/M2 cells and cultured in Myelocult H5100™ media (StemCell Technologies) along with different treatment. After 2 week of culture human cells were plated, in triplicate, in Methocult™ media (StemCell Technologies) for colony-forming and replating assays.

### Colony Assays

Following lentiviral transduction or in vitro culture with SL/M2 stroma, human cells were harvested, counted by trypan blue exclusion and 100-200 cells were plated per well of a 12-well plate in Methocultmedia. After 2 weeks, total colonies were counted and replated for secondary colony-formation assay.

### Statistical Analysis

Statistical analyses were performed with the aid of Microsoft Excel™, SAS 9.2™ and Graphpad Prism™ software as indicated in the figure legends. For differential gene expression RNA Seq analysis, we used DESeq™ (Li etal., 2014b) (version 1.6.1) in R (version 2.14.1), and identified differentially expressed genes (false discovery rate=10%).

### EXAMPLE 2

### RNA Editing Contributes to MDS Initiation and Maintenance in Inflammatory Microenvironments that Promote ADAR1 Activation

Previously, we reported that inflammatory cytokine-driven ADAR1 editing of RNA, primarily within Alu-containing transcripts, increased during malignant reprogramming of human pre-malignant myeloid progenitors into self-renewing leukemia stem cells (LSCs)². Lentiviral shRNA knockdown of ADAR1 inhibited serial transplantation suggesting that ADAR1 was required for LSC maintenance. Notably, ADAR1-activation in these pre-leukemic progenitors induced GSK3β missplicing, which prevented degradation of β-catenin—a self-renewal agonist. Because myeloid bone marrow disorders, such as myelodysplastic syndrome (MDS), usually arise during aging in inflammatory microenvironments, we examined RNA editing rates in young versus aged bone marrow HSPC. At known editing loci, adenosine (A)-to-inosine (I) changes, which are subsequently read as guanosines (G), increased in aged compared with young HPC.

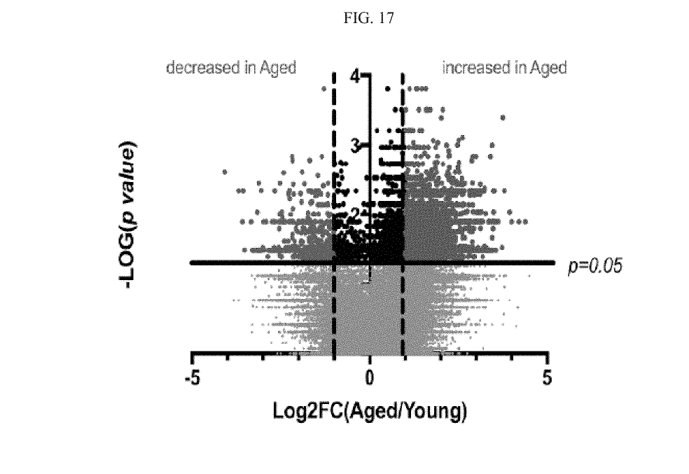

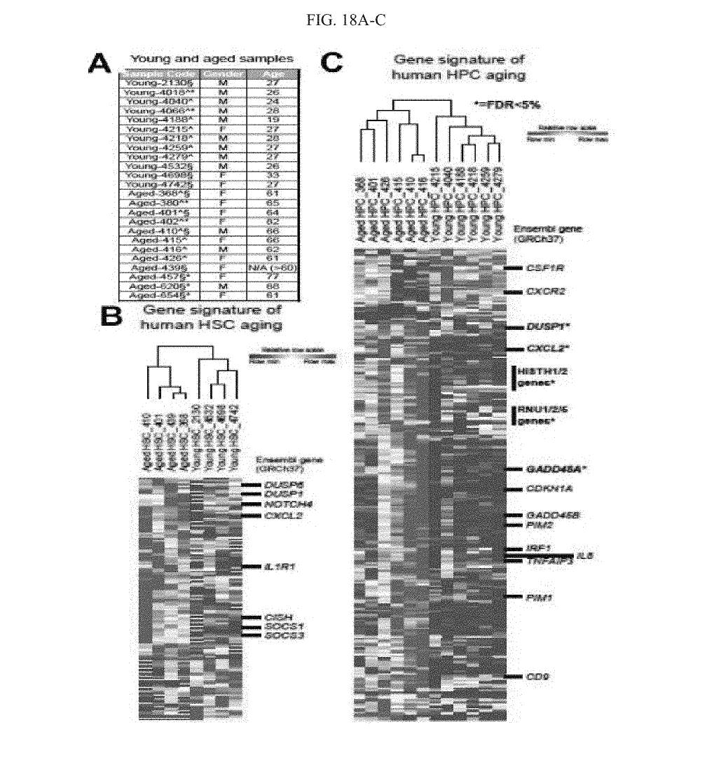

These data suggest that niche-dependent RNA editing deregulation contributes to normal HPC aging (FIG. 17). Notably, whole transcriptome RNA-seq analysis of 24 FACS-purified samples revealed that inflammatory cytokine receptors and downstream signaling pathway genes (IL1R1, CISH, SOCS1 and SOCS3) were upregulated during human HSC aging (FIG. 18A, B)¹². In aged human HPC, expression of DNA damage (GADD45A, GADD45B) genes was increased together with pro-inflammatory genes (CXCL2, IRF1, IL-8, TNFA1P3), which can activate ADAR1 (FIG. 18B, C). Thus, we will investigate the functional role of ADAR1 editase activity in HSPC aging.

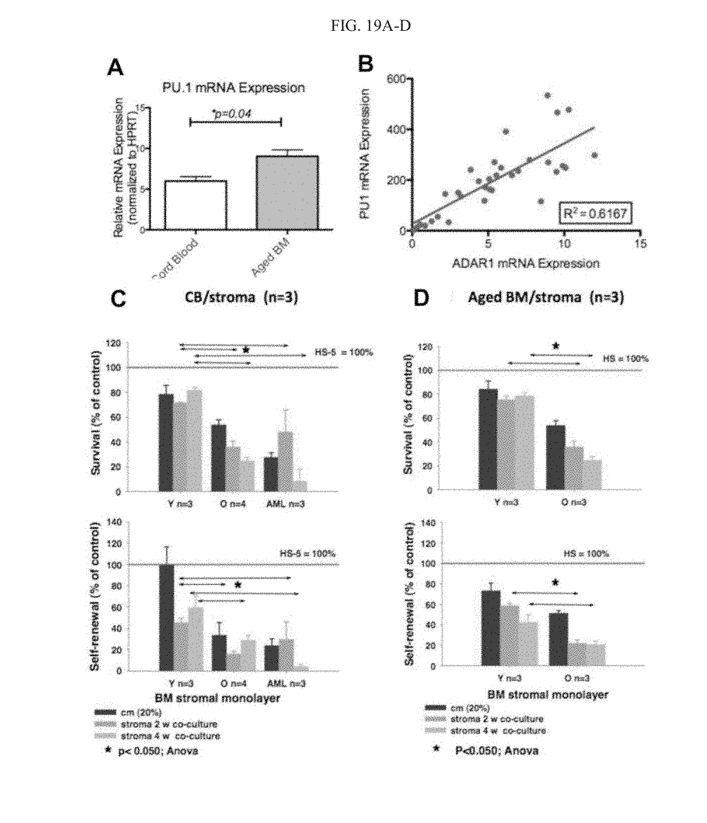

Our current results show that both survival and self-renewal of normal HSPC are impaired in the presence of aged or MDS/AML bone marrow stroma, indicating that cell-extrinsic factors derived from aged or diseased niches are crucial determinants of HSPC function and age-related myeloid disorders, such as MDS²⁶. Previous studies revealed that normal human hematopoiesis in the bone marrow skews toward the myeloid lineage with aging²⁷′²⁸. Similarly, we observed that normal aged bone marrow HSPC exhibited increased expression of PU.1 compared with their young progenitor counterparts (FIG. 19A). Lentiviral overexpression of ADAR1 in cord blood progenitors (CD34⁺CD38⁺Lin⁻) also induced PU.1 expression in colony assays. These studies suggested that adenosine-to-inosine (A-to-I) editing by ADAR1 may be directly involved in myeloid lineage priming typical of aging and MDS (FIG. 19B). Furthermore, we investigated the relative effects of young (greater than 35 years old) versus aged (greater than 65 years old) and AML bone marrow stroma on the survival and self-renewal of normal cord blood HSPC (FIG. 19C, D). Compared with HSPC co-cultured on a normal human bone marrow derived stromal cell line (HS), conditioned media (CM) derived from, or co-culture with, old or AML stroma impaired normal HSPC maintenance thereby underscoring the stem cell regulatory importance of the niche (FIG. 19C, D).

As a clonal bone marrow disorder, MDS predominantly affects elderly adults (average age greater than 65 years) and is typified by ineffective hematopoiesis resulting in peripheral blood cytopenias. Somatic mutations in epigenetic modifier genes (e.g. DNMT3A, EZH2, TET2), RNA splicing factors (e.g. SF3B1, SRSF2) as well as transcription factors and co-repressors (e.g. RUNX1, TP53) promote MDS initiation^{19,39}. Moreover, mesenchymal stromal cell (MSC)-derived inflammatory cytokines and β-catenin signaling in the bone marrow niche contribute to MDS HSPC maintenance^{9,40}.

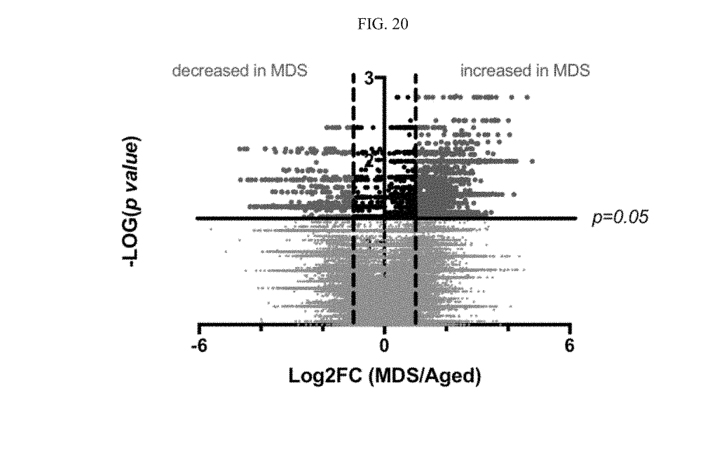

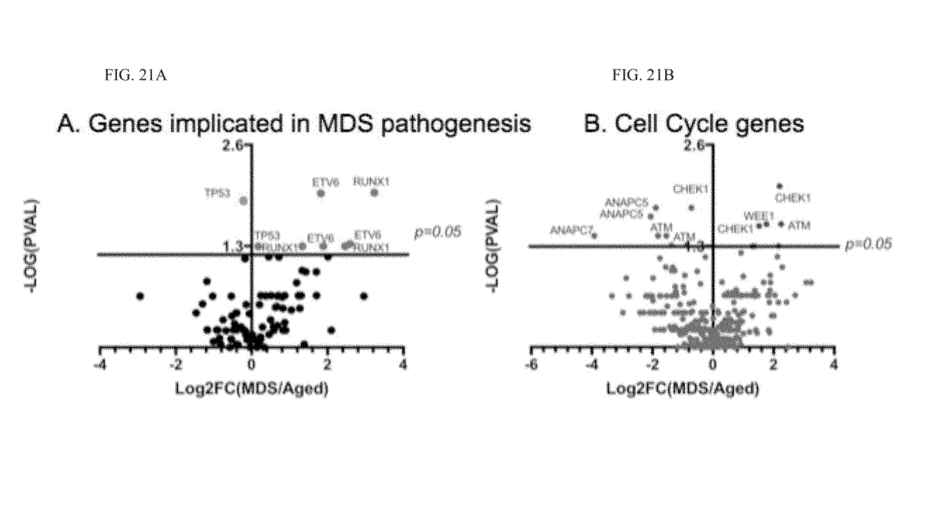

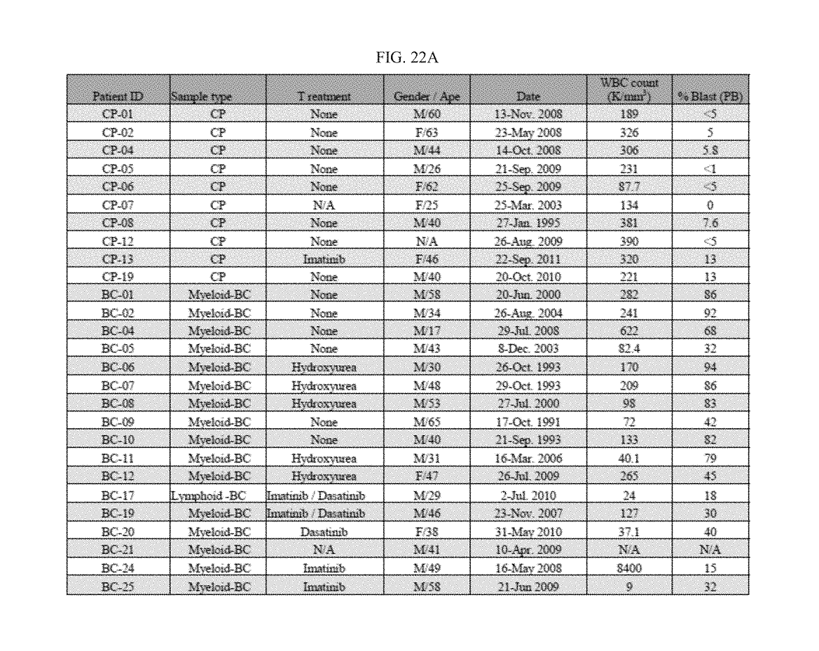

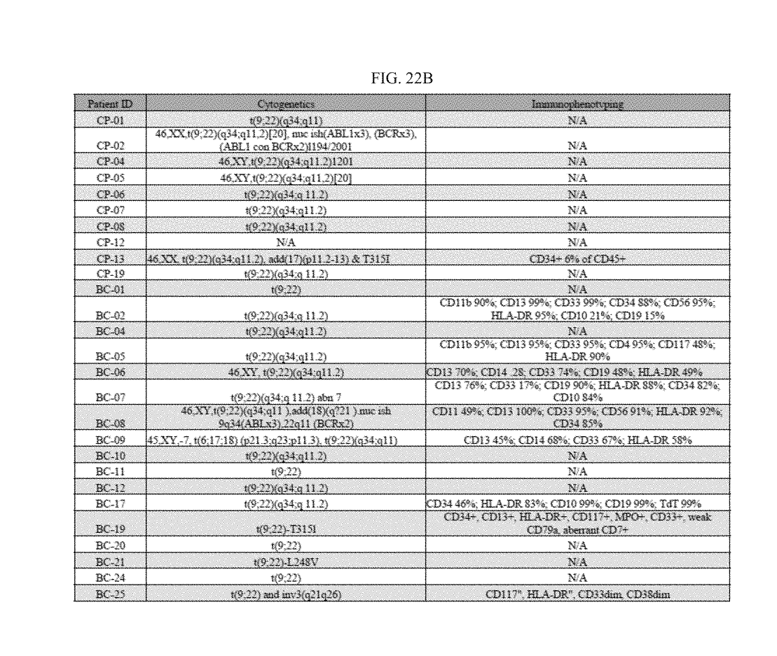

Previously, we discovered that β-catenin activation occurred following ADAR1-dependent GSK3β missplicing in human pre-malignant myeloid progenitors. To investigate whether inflammatory-cytokine activated ADAR1 contributes to MDS pathogenesis, we evaluated RNA editing levels in FACS purified MDS compared with normal aged HPC by RNA-seq. Preliminary data showed increased A-to-I RNA editing in MDS compared with aged HPC (FIG. 20). A closer look at the editome of commonly deregulated genes in MDS¹⁹ revealed increased editing efficiency (% G) and number of editing sites in ETV6 (3 editing sites) and RUNX1 (3 editing sites) transcripts (FIG. 21A). Another transcript affected by A-to-I editing, TP53, is often mutated in early phase MDS and is strongly associated with MDS patient survival^{41,42}. Interestingly, there are two significantly differentially edited sites in TP53 transcript, with either increased or decreased editing efficiency compared to normal aged samples (FIG. 21A). This suggests that RNA editing may contribute to MDS initiation and maintenance in inflammatory microenvironments that promote ADAR1 activation. With regard to ADAR1's impact on cell cycle regulatory transcripts, we observed enhanced editing of CHEK1 and WEE1 (DNA damage induced cell cycle regulator), as well as ATM (regulator of p53 and BRCA1) transcripts (FIG. 21B). Interestingly, reduced editing is seen at different sites of ATM and CHEK1, suggesting that ADAR1 editing location may be distinct in MDS progenitors compared to normal aged samples. Finally, A-to-I editing has been shown to stabilize endothelial cell cathepsinS (CTSS) transcripts and aberrant endothelial cell sprouting, which could promote MDS HSPC maintenance¹⁶.

### REFERENCES

Abrahamsson, et al. (2009). Glycogen synthase kinase 3beta missplicing contributes to leukemia stem cell generation. PNAS USA Vol. 106, 3925-3929.

Bahn, et al. (2015). Genomic analysis of ADAR1 binding and its involvement in multiple RNA processing pathways. Nature communications 6, 6355.

Blahna, et al. (2012). Smad-mediated regulation of microRNA biosynthesis. FEBS Lett 586, 1906-1912.

Chawla, et al. (2014). ADAR mediates differential expression of polycistronic microRNAs. Nucleic acids research 42, 5245-5255.

Burns, M. B. et al. APOBEC3B is an enzymatic source of mutation in breast cancer. Nature 494, 366-370, doi:10.1038/nature11881 (2013).

Chen, L., Li, Y, Lin, C. H., Chan, T. H., Chow, R. K., Song, Y, Liu, M., Yuan, Y. F., Fu, L., Kong, K. L., et al. (2013). Recoding RNA editing of AZIN1 predisposes to hepatocellular carcinoma. Nature medicine 19, 209-216.

Cheng, et al. (2015). Microarray expression profile of long non-coding RNAs in EGFR-TKIs resistance of human nonsmall cell lung cancer. Oncology reports 33, 833-839.

Claus, et al. (2012). Quantitative analyses of DAPK1 methylation in AML and MDS. International journal of cancer Journal international du cancer 131, E138-142.

Copley, et al. (2013). The Lin28b-let-7-Hmga2 axis determines the higher self-renewal potential of fetal haematopoietic stem cells. Nature cell biologyl5, 916-925.

Crews, et al. (2015). An RNA editing fingerprint of cancer stem cellreprogramming. Journal of translational medicine 13, 52.

Danielson, et al. (2015). Limited miR-17-92 overexpression drives hematologic malignancies. Leukemia research 39, 335-341.

Davis, et al. (2008). SMAD proteins control DROSHA-mediated microRNA maturation. Nature 454, 56-61.

Druker, et al. (1996). Effects of a selective inhibitor of the Abltyrosine kinase on the growth of Bcr-Ab1 positive cells. Nature medicine 2, 561-566.

Eades, G., Zhang, Y. S., Li, Q. L., Xia, J. X., Yao, Y., and Zhou, Q. (2014). Long non-coding RNAs in stem cells and cancer. World journal of clinical oncology 5, 134-141.

Flicek, et al. (2010). Ensembl's 10th year. Nucleic acids research 38, D557-562.

Essers, et al. (2009). IFNalpha activates dormant haematopoietic stem cells in vivo. Nature 458, 904-908.

Fialkow, et al. (1977). Chronic myelocytic leukemia: clonal origin in a stem cell common to the granulocyte, erythrocyte, plateletand monocyte/macrophage. The American journal of medicine 63, 125-130.

Fumagalli, et al. (2015). Principles Governing A-to-I RNA Editing in the Breast Cancer Transcriptome. Cell reports 13, 277-289.

George, C. X., et al. (2008). Organization of the mouse RNAspecific adenosine deaminase Adar1 gene 5′-region and demonstration of STAT1-independent, STAT2-dependent transcriptional activation by interferon. Virology 380, 338-343.

George, C. X., et al. (2015). STAT2-dependent induction of RNA adenosine deaminase ADAR1 by type I interferon differs between mouse and human cells in the requirement for STAT1. Virology 485, 363-370.

Geron, et al. (2008). Selective inhibition of JAK2-driven erythroid differentiation of polycythemia vera progenitors. Cancer cell 13, 321-330.

Goff, et al. (2013). A Pan-BCL2 inhibitor renders bone-marrow-resident human leukemia stem cells sensitive to tyrosine kinase inhibition. Cell stem cell 12, 316-328.

Gommans, W. M., et al. (2010). A mammalian reporter system for fast and quantitative detection of intracellular A-to-I RNA editing levels. Analytical biochemistry 399, 230-236.

Guenzl, P. M., and Barlow, D. P. (2012). Macro lncRNAs: a new layer of cis-regulatory information in the mammalian genome. RNA biology 9, 731-741.

Han, J., et al. (2014). Efficient in vivo deletion of a large imprinted lncRNA by CRISPR/Cas9. RNA biology 11, 829-835.

Han, et al. (2015). The Genomic Landscape and Clinical Relevance of A-to-I RNA Editing in Human Cancers. Cancer cell 28, 515-528.

Ho, T. T., et al. (2014). Targeting noncoding RNAs with the CRISPR/Cas9 system in human cell lines. Nucleic acids research.

Jiang, Q., Crews, L. A., and Jamieson, C. H. (2013). ADAR1 promotes malignant progenitor reprogramming in chronic myeloid leukemia. Proceedings of the National Academy of Sciences of the United States of America 110, 1041-1046.

Hartner, J. C., et al. (2009). ADAR1 is essential for the maintenance of hematopoiesis and suppression of interferon signaling. Nature immunology 10, 109-115.

Heldin, C. H., et al. (1997). TGF-beta signalling from cell membrane to nucleus through SMAD proteins. Nature 390, 465-471.

Hu, Y. H., et al. (2011). Aberrant protein expression and promoter methylation of p16 gene are correlated with malignant transformation of salivary pleomorphic adenoma. Archives of pathology & laboratory medicine 135, 882-889.

Huang da, W., et al. (2009). Systematic and integrative analysis of large gene lists using DAVID bioinformatics resources. Nature protocols 4, 44-57.

Jamieson, C. H., et al. (2004). Granulocyte-macrophage progenitors as candidate leukemic stem cells in blast-crisis CML. The New England journal of medicine 351, 657-667.

Jiang, Q., et al. (2013). ADAR1 promotes malignant progenitor reprogramming in chronic myeloid leukemia. Proceedings of the National Academy of Sciences of the United States of America 110, 1041-1046.

Karlic, H., et al. (2014). The role of epigenetics in the regulation of apoptosis in myelodysplastic syndromes and acute myeloid leukemia. Critical reviews in oncology/hematology 90, 1-16.

Kiran, A., and Baranov, P. V. (2010). DARNED: a DAtabase of RNa EDiting in humans. Bioinformatics 26, 1772-1776.

Li, H., and Durbin, R. (2009). Fast and accurate short read alignment with BurrowsWheeler transform. Bioinformatics 25, 1754-1760.

Li, X., et al. (2014). Effects of physicochemical properties of nanomaterials on their toxicity. Journal of biomedical materials research Part A.

Li, A., Zhang, J., and Zhou, Z. (2014a). PLEK: a tool for predicting long non-coding RNAs and messenger RNAs based on an improved k-mer scheme. BMC bioinformatics 15, 311.

Li, N., et al. (2014b). Cyclin C is a haplo insufficient tumour suppressor. Nature cell biology 16, 1080-1091.

Liddicoat, et al. (2008). The signaling protein Wnt4 enhances thymopoiesis and expands multipotent hematopoietic progenitors through beta-catenin-independent signaling. Immunity 29, 57-67.

Mariner, P. D., et al. (2008). Human Alu RNA is a modular transacting repressor of mRNA transcription during heat shock. Molecular cell 29, 499-509.

Melton, et al. (2010). Opposing microRNA families regulate self-renewal in mouse embryonic stem cells. Nature 463, 621-626.

Morin, R. D., et al. (2008). Application of massively parallel sequencing to microRNA profiling and discovery in human embryonic stem cells. Genome research 18, 610-621.

Mortazavi, et al. (2008). Mapping and quantifying mammalian transcriptomes by RNA-Seq. Nature methods 5, 621-628.

Mudunuri, U., et al. (2009). bioDBnet: the biological database network. Bioinformatics 25, 555-556.

Nakao, et al. (1997). TGF-betareceptor-mediated signalling through Smad2, Smad3 and Smad4. The EMBO journal 16, 5353-5362.

Nemlich, Y, et al. (2013). MicroRNA-mediated loss of ADAR1 in metastatic melanoma promotes tumor growth. Journal of clinical investigation 123, 2703-2718.

Nik-Zainal, S. et al. Association of a germline copy number polymorphism of APOBEC3A and APOBEC3B with burden of putative APOBEC-dependent mutations in breast cancer. Nature genetics 46, 487-491, doi:10.1038/ng.2955 (2014)

Nishikura, K. (2010). Functions and regulation of RNA editing by ADAR deaminases. Annual review of biochemistry 79, 321-349.

Ota, H., et al. (2013). ADAR1 forms a complex with Dicer to promote microRNA processing and RNA-induced gene silencing. Cell 153, 575-589.

Patil, V. S., Zhou, R., and Rana, T. M. (2014). Gene regulation by non-coding RNAs. Critical reviews in biochemistry and molecular biology 49, 16-32.

Patterson, M., et al. (2014). let-7 miRNAs can act through notch to regulate human gliogenesis. Stem cell reports 3, 758-773.

Picardi, E., et al. (2015). Profiling RNA editing in human tissues: towards the inosinome Atlas. Scientific reports 5, 14941.

Piskounova, E., et al. (2011). Lin28A and Lin28B inhibit let-7 microRNA biogenesis by distinct mechanisms. Cell 147, 1066-1079.

Qi, L., et al. (2014). RNA editome imbalance in hepatocellular carcinoma. Cancer research 74, 1301-1306.

Qian, J., et al. (2010). Methylation of DAPK1 promoter: frequent but not an adverse prognostic factor in myelodysplastic syndrome. International journal of laboratory hematology 32, 74-81.

Qin, Y. R., et al. (2014). Adenosine-to-inosine RNA editing mediated by ADARs in esophageal squamous cell carcinoma. Cancer research 74, 840-851.

Quintas-Cardama, A., et al. (2014). Predicting outcomes in patients with chronic myeloid leukemia at any time during tyrosine kinase inhibitor therapy. Clinical lymphoma, myeloma & leukemia 14, 327-334 e328.

Rampal, R., et al. (2014). DNA hydroxymethylation profiling reveals that WT1 mutations result in loss of TET2 function in acute myeloid leukemia. Cell reports 9, 1841-1855.

Raval, A., et al. (2007). Downregulation of death-associated protein kinase 1 (DAPK1) in chronic lymphocytic leukemia. Cell 129, 879-890.

Reya, T., et al. (2003). A role for Wnt signaling in self-renewal of haematopoietic stem cells. Nature 423, 409-414.

Roberts, S. A., et al. (2013). An APOBEC cytidine deaminase mutagenesis pattern is widespread in human cancers. Nature genetics 45, 970-976.

Sawyers, C. L. (2010). Even better kinase inhibitors for chronic myeloid leukemia. The New England journal of medicine 362, 2314-2315.

Shah, S. P., et al. (2009). Mutational evolution in a lobular breast tumour profiled at single nucleotide resolution. Nature 461, 809-813.

Solomon, 0., et al. (2013). Global regulation of alternative splicing by adenosine deaminase acting on RNA (ADAR). RNA 19, 591-604.

Soverini, S., de Benedittis, C., Mancini, M., and Martinelli, G (2015). Mutations in the BCR-ABL1 Kinase Domain and Elsewhere in Chronic Myeloid Leukemia. Clinical lymphoma, myeloma & leukemia 15 Suppl, 5120-128.

Tahira, et al. (2011). Long noncoding intronic RNAs are differentially expressed in primary and metastatic pancreatic cancer. Molecular cancer 10, 141.

Tallawi, et al. (2014). Poly(glycerol sebacate)/poly(butylene succinate-dilinoleate) (PGS/PBS-DLA) fibrous scaffolds for cardiac tissue engineering. Tissue engineering Part C, Methods.

Veliz, et al. (2003). Substrate analogues for an RNAediting adenosine deaminase: mechanistic investigation and inhibitor design. Journal of the American Chemical Society 125, 10867-10876.

Viswanathan, S. R., Daley, G Q., and Gregory, R. I. (2008). Selective blockade of microRNA processing by Lin28. Science 320, 97-100.

Viswanathan, et al. (2009). Lin28 promotes transformation and is associated with advanced human malignancies. Nature genetics 41, 843-848.

Wang, et al. (2015). The role of Lin28b in myeloid and mast cell differentiation and mast cell malignancy. Leukemia 29, 1320-1330. Wang, Q., Khillan, J., Gadue, P., and Nishikura, K. (2000). Requirement of the

RNA editing deaminase ADAR1 gene for embryonic erythropoiesis. Science 290, 1765-1768.

Wang, et al. (2004). Stress-induced apoptosis associated with null mutation of ADAR1 RNA editing deaminase gene. The Journal of biological chemistry 279, 4952-4961.

Wu, X., Liu, W., Tian, Y., Xiao, M., Wu, Y., and Li, C. (2011). Aberrant methylation of death-associated protein kinase 1 CpG islands in myelodysplastic syndromes. Acta haematologica 125, 179-185.

Yang, et al. (2006). Modulation of microRNA processing and expression through RNA editing by ADAR deaminases. Nature structural & molecular biology 13, 13-21.

Yildirim, et al. (2013). Xist RNA is a potent suppressor of hematologic cancer in mice. Cell 152, 727-742.

Zhou, et al. (2014a). Prioritizing candidate disease-related long non-coding RNAs by walking on the heterogeneous lncRNA and disease network. Molecular bioSystems.

Yu, et al. (2007). let-7 regulates self renewal and tumorigenicity of breast cancer cells. Cell 131, 1109-1123.

Yu, et al. (2012). LncRNAs expression signatures of renal clear cell carcinoma revealed by microarray. PloS one 7, e42377.

Zipeto, et al. (2015). RNA rewriting, recoding, and rewiring in human disease. Trends in molecular medicine 21, 549-559.

Zhou, et al. (2014b). Non-coding RNAs in epithelial immunity to Cryptosporidium infection. Parasitology 141, 1233-1243.

A number of embodiments of the invention have been described. Nevertheless, it can be understood that various modifications may be made without departing from the spirit and scope of the invention. Accordingly, other embodiments are within the scope of the following claims.

1. A method for:
treating, ameliorating, stopping or slowing the progression of, or preventing a cancer or a cancer associated with a stem cell,
inhibiting, decreasing or slowing the progression of a therapeutically responsive or a drug responsive cancer to a therapeutically resistant (drug resistant) cancer,
inhibiting, decreasing or slowing the generation of self-renewing leukemia stem cells (LSCs) or the maintenance of LSCs,
decreasing or inhibiting myelodysplastic syndrome (MDS) or a myeloproliferative neoplasm (MPN) initiation and/or maintenance in inflammatory microenvironments,
inhibiting or decreasing the amount of GSK3β missplicing and increasing degradation of β-catenin, and/or
enhancing let-7 microRNA (miRNA) biogenesis, decreasing adenosine-to-inosine (A-to-I) editing of polycistronic let-7 loci, and/or increasing levels of mature let-7 microRNA (miRNA) levels, comprising:
(a) administering to a subject in need thereof, or in need of treatment, an agent or combination of agents that inhibit or decrease the expression or activity of Janus kinase 2 (JAK2) and:
(i) breakpoint cluster region protein (BCR)-Abelson murine leukemia viral oncogene homolog 1 (ABL1), or BCR-ABL1 (a BCR-ABL fusion protein),
(ii) double-stranded RNA-specific adenosine deaminase ADAR1), or
(iii) ADAR1 and BCR-ABL1; or
(b) (i) providing an agent or combination of agents that inhibit or decrease the expression or activity of JAK2 and:
(1) BCR-ABL1;
(2) Adenosine Deaminase Acting on RNA1 (ADAR1); or
(3) ADAR1 and BCR-ABL1,
(ii) administering to a subject in need thereof, or in need of treatment, the agent or combination of agents of (b)(i), thereby:
treating, ameliorating, stopping or slowing the progression of, or preventing a cancer or a cancer associated with a stem cell,
inhibiting, decreasing or slowing the progression of a therapeutically responsive or a drug responsive cancer to a therapeutically resistant (drug resistant) cancer,
inhibiting, decreasing or slowing the generation of self-renewing leukemia stem cells (LSCs) or the maintenance of LSCs,
decreasing or inhibiting myelodysplastic syndrome (MDS) or a myeloproliferative neoplasm (MPN) initiation and/or maintenance in inflammatory microenvironments,
inhibiting or decreasing the amount of GSK3β missplicing and increasing degradation of β-catenin, and/or
enhancing let-7 microRNA (miRNA) biogenesis, decreasing adenosine-to-inosine (A-to-I) editing of polycistronic let-7 loci, and/or increasing levels of mature let-7 microRNA (miRNA) levels.

2. The method of claim 1, wherein the cancer or the cancer associated with a stem cell is:
(a) myelodysplastic syndrome (MDS) or a myeloproliferative neoplasm (MPN);
(b) lobular breast, hepatocellular or esophageal cancer.

3. The method of claim 1, wherein the efficacy (or success) of the method is assessed by the detection of:
a decrease in editing efficiency in (or the amount of adenosine-to-inosine (A-to-I) RNA editing of) pri-let-7 microRNA (miRNA) transcripts, or a decrease in the amount of ADAR1-mediated hyper-edited sites in pri-let-7 microRNAs, or
a decrease in the adenosine-to-inosine (A-to-I) RNA editing of apolipoprotein B mRNA-editing enzyme catalytic polypeptide-like 3 (APOBEC3).

4. The method of claim 1, wherein:
(a) the agent or combination of agents that inhibit or decrease the expression or activity of JAK2 comprise: ruxolitinib (or JAKAFI™, or JAKAVI™); lestaurtinib (or CEP-701); pacritinib (or SB-1518); SAR302503 (or TG101348, or N-tert-Butyl-3-{5-methyl-2-[4-(2-pyrrolidin-1-yl-ethoxy)-phenylamino]-pyrimidin-4-ylamino}-benzenesulfonamide); momelotinib (or CYT387, or N-(cyanomethyl)-4-{2-[4-(morpholin-4-yl)anilino]pyrimidin-4-yl}benzamide); AZD1480, or (S)-5-chloro-N2-(1-(5-fluoropyrimidin-2-yl)ethyl)-N4-(5-methyl-1H-pyrazol-3-yl)pyrimidine-2,4-diamine; XL019, or (S)-N-(4-(2-((4-morpholinophenyl)amino)pyrimidin-4-yl)phenyl)pyrrolidine-2-carboxamide; tofacitinib (also known as tasocitinib), or 3-((3R,4R)-4-methyl-3-(methyl(7H-pyrrolo[2,3-d]pyrimidin-4-yl)amino)piperidin-1-yl)-3-oxopropanenitrile, or XELJANZ™, or JAKVINUS™; NVP-BSK805, or 4-(2,6-difluoro-4-(3-(1-(piperidin-4-yl)-1H-pyrazol-4-yl)quinoxalin-5-yl)benzyl)morpholine; or, INCB16562, or 242,6-dichlorophenyl)-1,8-dihydroimidazo[4,5-d]dipyrido[2,3-b:4′,3′-f]azepine;
(b) the agent or combination of agents that inhibit or decrease the expression or activity of BCR-ABL1 comprise: imatinib (or GLEEVEC™, or GLIVEC™); nilotinib (or TASIGN™); dasatinib (or SPRYCEL™, or BMS-354825); bosutinib (or BOSULIF™); pornatinib (or ICLUSIG™, or AP24534); bafetinib or Benzamide, N-(3-((4,5′-bipyrimidin)-2-ylamino)-4-methylphenyl)-4-(((3S)-3-(dimethylamino)-1-pyrrolidinyl)methyl)-3-(trifluoromethyl)-;4-[[(3S)-3-Dimethylaminopyrrolidin-1-yl]methyl]-N-[4-methyl-3-[(4-pyrimidin-5-yl[pyrimidin-2-yl]amino]phenyl]-3-(trifluoromethyl)benzamide; or, a 1,3,4 thiadiazole derivative; or
(c) agent or combination of agents that inhibit or decrease the expression or activity of ADAR1 comprise agents or compositions as described in:
WO2013/036867 (PCT/US2012/054307), or U.S. Pat. No. 9,611,330; or WO2015/120197 (PCT/US2015/014686).

5. The method of claim 1, wherein:
(a) the agent or combination of agents that inhibit or decrease the expression or activity of JAK2, ADAR1 and/or BCR-ABL1 is or comprises:
(1) a nucleic acid,
(2) a peptide or polypeptide, or
(3) a small molecule, lipid, saccharide, nucleic acid or polysaccharide capable of inhibiting or decreasing the activity of a JAK2, ADAR1 and/or BCR-ABL1 protein, enzyme, transcript and/or gene;
(b) the compound or composition is formulated as a pharmaceutical composition, or is formulated for administration in vivo; or formulated for enteral or parenteral administration, or for oral, intravenous (IV) or intrathecal (IT) administration, wherein optionally the compound or formulation is administered orally, parenterally, by inhalation spray, nasally, topically, intrathecally, intrathecally, intracerebrally, epidurally, intracranially or rectally;
wherein optionally the formulation or pharmaceutical composition is contained in or carried in a nanoparticle, a particle, a micelle or a liposome or lipoplex, a polymersome, a polyplex or a dendrimer; or
(c) the compound or composition, or the formulation or pharmaceutical composition, is formulated as, or contained in, a nanoparticle, a liposome, a tablet, a pill, a capsule, a gel, a geltab, a liquid, a powder, an emulsion, a lotion, an aerosol, a spray, a lozenge, an aqueous or a sterile or an injectable solution, or an implant.

6. The method of claim 1, wherein the nucleic acid capable of inhibiting or decreasing the expression or activity of a JAK2, ADAR1 and/or BCR-ABL1 protein, enzyme, transcript and/or gene comprises or is contained in a nucleic acid construct or a chimeric or a recombinant nucleic acid, or an expression cassette, vector, plasmid, phagemid or artificial chromosome, optionally stably integrated into the cell's chromosome, or optionally stably episomally expressed, and optionally the cell is a cancer cell or a cancer cell line, or a carcinoma cell line or an immortalized cell line.

7. A kit comprising a compound or composition or a formulation or a pharmaceutical composition as used in a method of claim 1, and optionally comprising instructions on practicing a method of any one of the preceding claims.

8-10. (canceled)

11. A method for detecting leukemic progression into blast phase from chronic phase and a method for treating a blast phase leukemia comprising the steps of:
(a) determining if pri-let-7d levels are reduced as compared to a normal control or a previous sample from a patient while in chronic phase; or
(b)
(i) collecting a blood or serum sample from a patient with leukemia or an individual suspected of having leukemia;
(ii) isolating mononuclear cells from the blood sample;
(iii) isolating CD34+ cells;
(iv) isolating RNA from the CD34+ cells;
(v) converting the RNA from step (iv) into cDNA;
(vi) evaluating miRNA expression using MiScript qPCR array or equivalent; and
(vii) determining if pri-let-7d levels are reduced as compared to a normal control or a previous sample from the patient while in chronic phase,
wherein a reduction in pri-let-7d levels indicates that the patient is in or entering blast phase leukemia and should be treated or enrolled in a clinical trial,
and optionally, if the pri-let-7d levels are reduced the patient is treated with a combination of drugs or agents comprising: a JAK2 inhibitor, a BCR-ABL-1 inhibitor or a combination of the two; a JAK2 inhibitor, a ADAR1 inhibitor or a combination of the two; or the patient is treated with a combination of drugs as set forth in any of the preceding claims,
and optionally a reduction of pri-let-7d levels by at least between about 1% to 50%, or at least about 5% or 10%, is considered sufficient to administer the combination of drugs or agents, or is considered sufficient to indicate that the patient is in or entering blast phase leukemia.

12. A method for treating a patient in blast phase comprising the steps of:
(a) collecting a blood sample from a patient in blast phase;
(b) isolating mononuclear cells from the blood sample;
(c) isolating CD34+ cells
(d) isolating RNA from the CD34+ cells;
(e) converting the RNA from step (d) into cDNA;
(f) evaluating miRNA expression using MiScript qPCR array; and
(g) determining if pri-let-7d levels are reduced as compared to a normal control or a previous sample from the patient while in chronic phase,
wherein if the pri-let-7d levels are reduced the patient is treated with a JAK2 inhibitor, BCR-ABL-1 inhibitor or a combination of the two or the patient is treated with a JAK2 inhibitor, or ADAR1 inhibitor or a combination of the two.

13. A method for determining leukemic stem cell generation and/or MPN disease progression using editome signatures of APOBEC3F (A3F) and/or APOBEC3G (A3G) wherein the chronic phase (CP) chronic myeloid leukemia (CML) (or CP CML) and pre-leukemic progenitors or blast crisis (BC) phase have different adenosine-to-inosine (A-to-I) RNA editing signature in A3F and A3G transcripts as compared to a corresponding BC CML and sAML leukemic stem cell,
and optionally if at least 1° A, 5% or 10% of the A3F and A3G transcripts differ or if between about 1° A and 40% of the A3F and A3G transcripts differ, then a determination of leukemic stem cell generation and/or MPN disease progression can be made, or a progression from CP CML to pre-leukemic progenitors or blast crisis (BC) phase has been made.

14. A method for detecting edited and unedited RNA transcripts binding to ADAR1 protein comprising:
(a) immunoprecipitating an RNA transcript binding to a ADAR1 protein by Crosslinking Immunoprecipitation (CLIP) with an ADAR1 antibody; and
(b) sequencing the immunoprecipitated RNA transcript and determining how many, or quantifying, how many RNA transcripts are edited or unedited by ADAR1, and/or determining how the RNA transcripts are edited by ADAR1.

15. The method of claim 2, wherein the myeloproliferative neoplasm (MPN) is chronic myeloid leukemia (CML), a blast crisis (BC) myeloid leukemia (CML) (BC CML), or acute myeloid leukemia (AML),
and optionally the BC CML is a therapy resistant BC CML.

16. The method of claim 3, wherein the amount of A-to-I RNA editing is measured by RNA editing site specific qPCR (RESSqPCR).

17. The method of claim 3, wherein the method is considered efficacious or successful if the amount of A-to-I RNA editing, or the amount of ADAR1-mediated hyper-edited sites in pri-let-7 microRNAs, is decreased by at least between about 1% to 50%, or at least about 5% or 10%.

18. The method of claim 5, wherein the nucleic acid comprises an inhibitory nucleic acid.

19. The method of claim 17, wherein the inhibitory nucleic acid comprises: an RNAi inhibitory nucleic acid molecule, a double-stranded RNA (dsRNA) molecule, a microRNA (mRNA), a small interfering RNA (siRNA), an antisense RNA, a short hairpin RNA (shRNA), or a ribozyme capable of capable of inhibiting or decreasing the expression or activity of a JAK2, ADAR1 and/or BCR-ABL1 protein, enzyme, transcript and/or gene.

20. The method of claim 5, wherein the peptide or polypeptide is or comprises an antibody or fragment thereof or equivalent thereof, capable of specifically binding an JAK2, ADAR1 and/or BCR-ABL1, and is capable of inhibiting or decreasing the activity of a JAK2, ADAR1 and/or BCR-ABL1 protein, enzyme, transcript and/or gene.
